SEQUENCE LISTING (13 sequences)

SEQ ID NO: 1 - protein, 1368 aa, Artificial [sequence omitted]

SEQ ID NO: 2 - DNA, 25 bp, Artificial
  CRISPR RNA recognition sequence
  misc_feature (3)..(23): N at position 3 to position 23 is A, T, C, or G
ggnnnnnnnnnnnnnnnnnnnnngg

SEQ ID NO: 3 - DNA, 200 bp, Artificial
  Repair template
cagcccccactacagcaagaacatccgcaagaaggaccctatcctcctgctgcactggtggaaggagatattcgggacgatcctgctttgaatcgtggccacaacgtttatcgtgcgcaggcttttccatcctcagccccacagggtaagatgctctgtcaacctaatgtgcttccaagtggttgctgtgtaggaaacct

SEQ ID NO: 4 - DNA, 23 bp, Artificial
  Cas9/sgRNA genomic target sequence
cctgcgcacgatgaaggtcgtgg

SEQ ID NO: 5 - DNA, 23 bp, Artificial
  Cas9/sgRNA genomic target sequence
tagttcgggatcgccacatgagg

SEQ ID NO: 6 - DNA, 23 bp, Artificial
  Cas9/sgRNA genomic target sequence
agacatcgcccattgagcgaggg

SEQ ID NO: 7 - DNA, 23 bp, Artificial
  Cas9/sgRNA genomic target sequence
tgtagcggatcacttagggctgg

SEQ ID NO: 8 - DNA, 23 bp, Artificial
  Cas9/sgRNA genomic target sequence
gccccagctctgcagcagggagg

SEQ ID NO: 9 - DNA, 23 bp, Artificial
  Cas9/sgRNA genomic target sequence
tgggctcgtgaagcatgtggggg

SEQ ID NO: 10 - DNA, 23 bp, Artificial
  donor plasmid
tagttcgggatcgccacatgagg

SEQ ID NO: 11 - DNA, 23 bp, Artificial
  donor plasmid
cctcatgtggcgatcccgaacta

SEQ ID NO: 12 - DNA, 23 bp, Artificial
  genome plasmid
cctcatgtggcgatcccgaacta

SEQ ID NO: 13 - DNA, 23 bp, Artificial
  genome plasmid
tagttcgggatcgccacatgagg

(19) United States
(12) Patent Application Publication
(10) Pub. No.: US 2022/0411826 A1
(43) Pub. Date: Dec. 29, 2022

(54) CO-OPTING REGULATORY BYPASS REPAIR OF GENETIC DISEASES

(71) Applicant: The Penn State Research Foundation, University Park, PA (US)

(72) Inventors: Douglas R. Cavener, University Park, PA (US); Jingjie Hu, University Park, PA (US); Barbara C. McGrath, University Park, PA (US); Rebecca A. Bourne, University Park, PA (US)

(21) Appl. No.: 17/845,447

(22) Filed: Jun. 21, 2022

Related U.S. Application Data
(60) Provisional application No. 63/212,752, filed on Jun. 21, 2021.

(51) Int. Cl. C12N 15/90

(52) U.S. Cl. CPC C12N 15/907; C12N 2800/80; C12N 2310/20

(57) ABSTRACT

The present application discloses methods of correcting a gene defect in a cell, methods of treating a patient having a disease or disorder characterized by a gene defect, methods of preparing a chimeric antigen receptor T cell, as well as systems for correcting a gene defect in a cell, ex vivo modified cells, and related compositions.

This application claims the benefit of U.S. Provisional Patent Application Ser. No. 63/212,752, filed Jun. 21, 2021, which is hereby incorporated by reference in its entirety.

This invention was made with government support under Grant No. DK088140 awarded by the National Institutes of Health. The Government has certain rights in the invention.

### FIELD

The present disclosure is directed to a methods, systems, modified cells, and compositions related to co-opting regulatory bypass repair of genetic diseases.

### BACKGROUND

Conventional treatment of genetic diseases has relied upon long-term drug therapy or organ transplantation which necessitates the use of immunosuppressive drugs that lead to an increased risk of infections and cancer. Because these therapeutic approaches entail severe and debilitating side-effects, strategies to permanently repair the underlying genetic defect have been sought. Gene therapy was pioneered through the use of viral expression vectors to overcome gene deficiency (Wilson, J. M., Human Gene Therapy. Clinical Development, 30:47-49 (2019); Dunbar et al., Science, 359(6372) (2018); and Lundstrom, K. Diseases, 6(2):42 (2018)), either by overexpressing a wild-type cognate to the deficient gene or with a heterologous gene that leads to metabolic compensation. Major drawbacks of viral vector gene expression are a lack of normal temporal, spatial and quantitative gene regulation and continued expression of the mutant gene. The advent of CRISPR/Cas9 based technologies (Jinek et al., Science, 337:816-821 (2012); Cong et al., Science, 339:819-823 (2013); Mali et al., Science, 339:823-826 (2013); and Cho et al., Nat. Biotechnol., 31:230-232 (2013)) provided an immediate solution to the problems inherent in existing gene therapies, namely targeted correction of genetic disease-causing mutations. Expression of Cas9 endonuclease with a single guide RNA (sgRNA) in eukaryotic cells induces a double-strand break (DSB) at a target site in the genome. The DSB can be repaired by two major pathways: error-prone non-homologous end joining (NHEJ), and homology directed repair (HDR). Although the HDR pathway has been shown to repair genes precisely in mouse models of human disease (Yin et al., Nat. Biotechnol., 32:551-553 (2014); Yin et al., Nature Biotechnol., 34:328-333 (2016); Tran et al., Mol. Ther., 28(12):2621-2634 (2020); Ohmori et al., Sci. Rep., 7:4159 (2017); Wang et al., Blood, 133:2745-2752 (2019); Vagni et al., Front Neurosci., 13:945 (2019); and Cai et al., Sci. Adv., 5(4) (2019)), this pathway is dependent upon cellular homologous recombination functions that are only expressed during cell division. Therefore, HDR is not capable of gene repair in post-mitotic cells (Cox et al., Nat. Med., 21:121-131 (2015) and Panier et al., Nat. Rev. Mol. Cell Biol., 14:661-672 (2013)). Base editing approaches (Komor et al., Nature, 533:420-424 (2016); Gaudelli et al., Nature, 551:464-471 (2017); Yeh et al., Nat. Commun., 9:2184 (2018); and Villiger et al., Nat. Med., 24:1519-1525 (2018)) provide precise genome editing in post-mitotic tissues, but both HDR and base editing are limited because the components provided in trans must be engineered and tested for each specific mutation. Given that many single-gene genetic diseases (Bansal et al., BMC Med., 15:213 (2017); Rebbeck et al., Hum. Mutat., 39:593-620 (2018); Julier et al., Orphanet J. Rare Dis., 5:29 (2010)) may be caused by a spectrum of mutations throughout the coding sequence, a gene therapy method that utilizes a single design to repair any one of several possible mutations would be highly advantageous.

The present disclosure is directed to overcoming these and other deficiencies in the art.

### SUMMARY

A first aspect relates to a method of correcting a gene defect in a cell. The method includes:

providing in a cell having a gene defect (i) a chimeric Clustered Regularly Interspaced Short Palindromic Repeats (CRISPR) associated (Cas) protein or a first nucleic acid molecule encoding the Cas protein, (ii) a guide RNA that is capable of base-pairing with a region of the defective gene between a promoter and a coding sequence thereof, or a second nucleic acid encoding the guide RNA, and (iii) a DNA template including a replacement coding sequence, which encodes a non-defective protein, and a transcription terminator sequence,

wherein upon binding of the guide RNA to the 5′ untranslated region of the defective gene and cleavage of the 5′ untranslated region by the Cas protein, the DNA template is inserted into the genome of the cell via non-homologous end-joining (NHEJ) repair pathway to allow for expression of the non-defective protein under control of the promoter while simultaneously blocking the expression of the defective gene, thereby correcting the gene defect.

A second aspect relates to a method of treating a patient having a disease or disorder characterized by a gene defect. The method includes:

repairing the gene defect in one or more cell types that express the defective gene product, said repairing including introducing into the one or more cell types (i) a Cas protein or a first nucleic acid molecule encoding the Cas protein, (ii) a guide RNA that is capable of base-pairing with a region of the defective gene between a promoter and a coding sequence thereof, or a second nucleic acid encoding the guide RNA, and (iii) a DNA template including a replacement coding sequence, which encodes a non-defective protein, and a transcription terminator sequence,

wherein upon binding of the guide RNA to the region of the defective gene and cleavage of that region by the Cas protein, the DNA template is inserted into the genome of the cell via NHEJ repair pathway to allow for expression of the non-defective protein under control of the promoter while simultaneously blocking the expression of the defective gene, thereby treating the disease or disorder.

A third aspect relates to a system for correcting a gene defect in a cell. The system includes:

a first vector that comprises a first nucleic acid molecule encoding a Cas protein;

a second vector that comprises a DNA template including a replacement coding sequence, which encodes a non-defective protein, and a transcription terminator sequence,

wherein one of the first and second vectors comprises a nucleic acid molecule encoding a guide RNA that is capable of base-pairing with a region of a defective gene between a promoter and a coding sequence thereof.

A fourth aspect relates to system for correcting a gene defect in a cell. The system includes:

one or more non-viral delivery vehicles that comprise a Cas protein, or a nucleic acid molecule encoding the Cas protein, a guide RNA that is capable of base-pairing with a region of a defective gene between a promoter and a coding sequence thereof, and a DNA template including a replacement coding sequence, which encodes a non-defective protein, and a transcription terminator sequence.

A fifth aspect relates a composition including a system as described herein.

A sixth aspect relates to an ex vivo modified cell prepared according to the methods described herein.

A seventh aspect relates to an ex vivo modified cell having a repair of a gene defect, the modified cell including a promoter and a coding sequence for a defective gene product, and a replacement coding sequence and transcription terminator inserted into a region between the promoter and the coding sequence for the defective gene product via NHEJ repair pathway, whereby the modified cell expresses a non-defective protein encoded by the replacement coding sequence under control of the promoter but not the defective gene product.

An eighth aspect relates to a composition including an aqueous delivery vehicle and the ex vivo modified cell according to any of those described herein.

A ninth aspect relates to a method of preparing a chimeric antigen receptor T cell. The method includes:

providing in an isolated T cell (i) a Cas protein or a first nucleic acid molecule encoding the Cas protein, (ii) a guide RNA that is capable of base-pairing with a region of a native gene between a promoter and a coding sequence thereof, or a second nucleic acid encoding the guide RNA, and (iii) a DNA template including a replacement coding sequence, which encodes a heterologous antigen receptor, and a transcription terminator sequence,

wherein upon binding of the guide RNA to a 5′ untranslated region of the native gene and cleavage of the 5′ untranslated region by the Cas protein, the DNA template is inserted into the genome of the cell via NHEJ repair pathway to allow for expression of the heterologous antigen receptor under control of the native gene promoter while simultaneously blocking the expression of the native gene product.

A tenth aspect relates to an ex vivo modified T cell prepared according to any method described herein.

An eleventh aspect relates to an ex vivo modified T-cell that expresses a chimeric antigen receptor, the modified T cell including a promoter and a coding sequence for native gene product, and a replacement coding sequence and transcription terminator inserted into a region between the promoter and the coding sequence for the native gene product via NHEJ repair pathway, whereby the modified T cell expresses a chimeric antigen receptor encoded by the replacement coding sequence under control of the promoter but not the native gene product.

A twelfth aspect relates to a composition including an aqueous delivery vehicle and the ex vivo modified T-cell as described herein.

With the development of CRISPR/Cas9-mediated gene editing technologies, correction of disease-causing mutations has become possible. However, current gene correction strategies preclude mutation repair in post-mitotic cells of human tissues, and a unique repair strategy must be designed and tested for each and every mutation that may occur in a gene. Here, a novel gene correction strategy, Co-opting Regulation Bypass Repair (CRBR), is developed, which can repair a spectrum of mutations in mitotic or post-mitotic cells and tissues. CRBR utilizes the NHEJ pathway to insert a coding sequence (CDS) and transcription/translation terminators targeted upstream of any CDS mutation and downstream of the transcriptional promoter. CRBR results in simultaneous co-option of the endogenous regulatory region and bypass of the genetic defect. CRBR is based on the efficient NHEJ repair pathway that is induced upon CRISPR/Cas9-mediated targeted DSB. Normally, NHEJ DSB repair results in the rejoining of two genomic DNA fragments cut by Cas9. However, Suzuki and coworkers (Suzuki et al., Nature, 540:144-149 (2016), which is hereby incorporated by reference in its entirety) have shown that NHEJ repair pathway can ligate heterologous DNA to the two cut ends generated by sgRNA/Cas9 double-strand cleavage. This mechanism, denoted as homologous-independent targeted insertion (HITI), can be used to insert large DNA fragments. The HITI method was used to develop CRBR as a novel gene therapy strategy whereby an entire CDS and transcription/translation terminator cassette is inserted downstream of a gene's promoter but upstream of a deleterious disease-causing mutation. Expression of the CRBR cassette, which contains the normal coding sequence of the gene being repaired, can rescue its deficiency by restoring normal expression of the wild-type CDS under its native promoter and other regulatory elements while bypassing the downstream mutated region. Because a single CRBR CDS-terminator cassette contains all of the wild-type coding sequence, it can therefore be used to rescue any coding sequence mutation, as well as splice-site mutations.

The. Additionally, a CRBR GFP-terminator cassette was integrated downstream of the human insulin promoter in cadaver pancreatic islets of Langerhans which resulted in insulin promoter regulated expression of GFP, demonstrating the potential utility of CRBR in human tissue gene repair.

To test the efficacy of CRBR, two genes were targeted, eukaryotic translation initiation factor 2 alpha kinase 3 (PERK) and insulin (INS), which are both critically important for pancreatic beta cell functions and maintenance of glucose homeostasis. In the first example, a mouse model of Wolcott-Rallison syndrome (WRS) was used, which presents with permanent neonatal diabetes due to the mutations in the PERK gene. Using CRBR, a complete PERK CDS-terminator cassette was successfully integrated into the 5′UTR and showed that its expression rescued two independent Perk KO alleles in mice, one with a large three-exon deletion and the other with a nonsense mutation. Notably, all of the severe anomalies (Harding et al., Molecular Cell, 7:1153-1163 (2001) and Zhang et al., Molecular and Cellular Biology, 22:3864-3874 (2002), both of which are hereby incorporated by reference in their entirety) including neonatal diabetes, growth retardation, necrotic death of the exocrine pancreas, and skeletal dysplasia were absent in the CRBR allele rescued Perk KO mice. The potential of CRBR for human gene therapy was also demonstrated by integrating a GFP CDS-terminator cassette downstream of the human insulin gene by both plasmid transfection and AAV transduction of human cadaver islets. A large number of pancreatic beta cells were observed within these islets that expressed high levels of GFP driven by the insulin promotor. The CRBR gene repair may be used in the future as the basis for a strategy to correct deficiencies in genes critical for insulin synthesis and secretion by autologous cell-tissue replacement therapy.

### BRIEF DESCRIPTION OF THE DRAWINGS

FIGS. 1A-1E show CRBR-mediated in vitro partial PERK CDS integration in Perk KO cell line. FIG. 1A depicts a schematic of CRBR strategy. The CDS-terminator cassette is flanked by Cas9/gRNA target sites in reverse orientation of the genome. Correct integration of the CRBR cassette is expressed under the native promoter, with the 5′UTR region having small changes resultant from residue target site from the donor. Salmon pentagon: PAM site (3 nt). Rectangle with gradient: Cas9/gRNA targeted protospacer sequence (20 nt); Cas9 cleavage locates at 17 nt to the white side, 3 nt to the side. 5′UTR-g: 5′UTR region in the genome. 5′UTR-d: 5′UTR region engineered in the donor. FIG. 1B shows a schematic of CRBR-Partial-CDS strategy for Perk^{Δex7-9/Δex7-9} genome. The donor plasmid provides a 3′intron6-rPERKex7-17CDS-bGHpA cassette that is flanked by Cas9/gRNA target sites in reverse orientation (5′ 20 nt-NGG 3′, SEQ ID NO: 10) as identified within the mPerk intron 6 (5′ CCN-20 nt 3′, SEQ ID NO: 11). Expression of Cas9 and mPERKin6-sgRNA leads to the cleavage of the mPerk-in6 cut sites (SEQ ID NOS: 12 and 13) that are engineered in the donor to generate the CRBR cassette, and also a targeted DSB at genomic mPerk intron 6. Correct integration of the CRBR cassette is retained while the incorrect integrant is prone to Cas9 excision. Small changes at 5′ junction should be spliced out with intron 6 and mature transcript results in a chimeric mouse-rat Perk sequence. FIGS. 1C and 1D show that Perk^{Δex7-9/Δex7-9} MEF cells (3×10⁶ cells) were electroporated with 1.8 μg of pX459-mPERKin6sg, 1.6 μg of rPERKex7-17-2cut donor or both in 100 μL using MEF 2 Nucleofector Kit. Puromycin (1 μg/mL) was used to enrich transfected cells (with pX459-mPERKin6sg treatment) for 3 days. Genomic DNA (FIG. 1C) was harvested 6 d post-transfection for 5′ and 3′ junction diagnostic PCRs. Primers were designed to flank the junction sites (triangle mark: 5′, 254 bp; 3′, 890 bp). Chimeric mouse-rat Perk mRNA expression levels (FIG. 1D) were quantified in sub-cultured Perk^{Δex7-9/Δex7-9} (PKO) MEF cells (mixed cell population). Relative gene expression was normalized to mActin first and then to PKO MEF cells. Quantification represents n=3 per treatment. Data are represented as mean±SE. Statistical significance was calculated relative to the no treatment control, pX459-mPERKin6sg only and rPERKex7-17-2cut donor only; *p<0.05, **p<0.01. FIG. 1E shows that protein expression levels were quantified in Perk^{+/+} (WT) and Perk^{Δex7-91/Δex7-9} (PKO) MEF cells and the CRBR-edited cell line #3(Perk^{CRBR-rPERKex7-17/backbone integration}) treated with 1 μM thapsigargin (Tg) for 4 hrs. Relative protein expression was normalized to eIF2α first and then to WT MEF cells. Quantification represents n=4 per cell line. Data are represented as mean±SE. Statistical significance was calculated relative to the Perk WT or Perk^{Δex7-9/Δex7-9} MEF cells; **p<0.01, ***p<0.001, n.s., not significant.

FIGS. 2A-2B depict CRBR-mediated in vitro full PERK CDS integration in Perk KO cell line. FIG. 2A shows a schematic of CRBR-Full-CDS strategy. The donor plasmid provides a full rPERKmyc CDS-bGHpA cassette that is flanked by a wild-type 5′UTR of mPerk and a Cas9/gRNA target site in reverse orientation as identified within the mPerk 5′UTR. Expression of Cas9 and mPERKutr5-sgRNA leads to the cleavage of the mPerk-utr5 cut sites that are engineered in the donor to generate the CRBR cassette, and also a targeted DSB at genomic mPerk 5′UTR. Correct integration of the CRBR cassette preserves the wild-type sequence of mPerk 5′UTR but also resumes the mPerk-utr5 cut site making it prone to excision. Small indels could retain the integration of the rPERKmyc CRBR cassette and no splicing is required to achieve a mature transcript of rat Perk from the CRBR-edited genome. FIG. 2B shows that Perk^{C528X/C528X} MEF cells (1×10⁵ cells) were electroporated with 1 μg of pX459-mPERKutr5sg, 1 μg of rPERKmyc-2cut donor or both using the 10 μL Neon transfection system in two replicates. Genomic DNA was harvested 2d post-transfection for 5′ and 3′ junction diagnostic PCRs. Primers were designed to flank the junction sites (triangle mark: 5′, 921 bp; 3′, 857 bp). The lower molecular weight bands seen in one replicate reflect that part of the CRBR-edited alleles had large NHEJ deletions at the junction.

FIGS. 3A-3E depict that CRBR-edited Perk allele rescues Perk KO allele in a proof-of-concept mouse model. FIG. 3A shows a schematic of rPERK-CRBR allele (in a wild-type mouse Perk background) from the transgenic mouse. FIG. 3B shows that blood glucose levels were monitored at P21, P28, and P42 of mice with genotypes indicated in the chart. Normal blood glucose levels were observed in Perk^{C528X/rPERK-CRBR} mice at all ages. Data are represented as mean±SE. Student's t-test showed no significant difference in blood glucose between C528X/CRBR mice (n=7) and littermate +/CRBR mice (n=5) or independent litters with at least one wild-type mouse Perk allele (C528X/+ or +/+, n=6) at all three age points. Only Perk KO mice (C528X/C528X, red, n=8) become diabetic before P28 and exceeded the glucometer upper limit (600 mg/dL) by P35. C528X/CRBR or +/CRBR mice were offspring from Perk^{+/rPERK-CRBR} crossed to Perk^{C528X/+} mice. Perk KO (C528X/C528X) or littermates (C528X/+ or +/+) were offspring from Perk^{C528X/+} mice intercross. FIG. 3C depicts representative Hematoxylin and Eosin staining images from the pancreas of Perk^{+/+} (P62), Perk^{C528X/+} (P53), Perk^{C528X/C528X} (P34), Perk^{C528X/rPERK-CRBR} (P46), and Perk^{rPERK-CRBR/tPERK-CRBR} (P46) mice. The Perk^{C528X/C528X} pancreas had typical Perk KO defects such as very small islets with reduced beta cell mass. The disorganized acinus structure contained some degranulated cells (white), clear halos around the nuclei, and gaps between acinar cells, which were not seen in the pancreas of the Perk^{C528X/rPERK-CRBR} and Perk^{rPERK-CRBR/rPERK-CRBR} mice. Bright field, 20× objective; scale bar, 100 μm. FIG. 3D shows that the mRNA expression levels of endogenous mPerk and rPerk from CRBR-edited allele in pancreas and brain of adult mice (1- to 5-month) were quantified using mPerk- and rPerk-specific primers and were normalized to mActin. Perk^{+/+}, n=6; Perk^{C528X/+}, n=6; Perk^{C528X/rPERK-CRBR}, n=9; Perk^{+/rPERK-CRBR}, n=7; Perk^{rPERK-CRBR/rPERK-CRBR}, n=8. Perk^{+/+} and Perk^{C528X/+} mice had no detectable rPerk signal (Ct value >36, used 40 for calculation if undetermined) in pancreas and brain. Data are represented as mean±SE. FIG. 3E shows two replicate mice with the same genotype that were sacrificed at P38 (Perk^{+/+}, from Perk^{+/rPERK-CRBR} intercross), P58 and P30 (Perk^{C528X/+}, from Perk^{C528X/+} cross Perk^{C528X/rPerk-CRBR}), and P46 (Perk^{C528X/rPERK-CRBR}, Perk^{+/rPERK-CRBR}, and Perk^{rPERK-CRBR/rPerk-CRBR}, from Perk^{C528X/rPERK-CRBR} cross Perk^{+/rPERK-CRBR}) Both mPERK and rPERK protein expression in pancreas were detected by immunoblotting using an anti-PERK antibody. The rPERK-myc protein was also recognized by a myc tag antibody. Solid triangle marks the true myc signal while the hollow triangle marks a nonspecific band recognized by the myc tag antibody. Negative control was Perk^{Δex7-9/Δex7-9} (PKO) MEF cells. Positive control was Perk^{+/+} (WT) MEF cells treated with or without 1 μM thapsigargin (Tg) for 4 hrs. Relative rPERK-myc protein expression was normalized to Actin first and then obtained by background subtraction of the average signal of the two Perk^{+/+} replicates.

FIGS. 4A-4E show CRBR-mediated in vitro EGFP CDS integration in mouse Ins2 gene. FIG. 4A shows a schematic of CRBR-EGFP-2cut strategy for wild-type mIns2 genome. The donor plasmid provides an EGFP CDS-pA cassette that is flanked by Cas9/gRNA target sites in reverse orientation (5′ 20 nt-NGG 3′) as identified within the mIns2 5′UTR in exon 1 (5′ CCN-20 nt 3′). No mIns2 5′UTR sequence is engineered between the 5′ cut site and the start codon of EGFP. Expression of Cas9 and mINS2utr5-sgRNA leads to the cleavage of the mIns2-utr5 cut sites that are engineered in the donor to generate the CRBR cassette as well as a targeted DSB at genomic mIns2 5′UTR. Correct integrants will retain the CRBR cassette while incorrect integrants are prone to excision. FIGS. 4B and 4C shows that MIN6 cells (1×10⁶ cells) were electroporated with 1 μg of EGFP-2cut donor with or without 1 μg of pX459-mINS2utr5sg in 100 μL using Nucleofector V Kit in two replicates. Cells were imaged (FIG. 4B) as live cultures 2d, 6d, and 15d post-transfection at 10× objective; scale bar, 100 μm. Genomic DNA (FIG. 4C) was harvested 6d post-transfection for 5′ and 3′ junction diagnostic PCRs. Primers were designed to flank the junction sites (solid triangle: 5′, 452 bp; 3′, 690 bp). The hollow triangle marks a nonspecific band recognized by 5′ junction PCR primers. FIGS. 4D and 4E show that EGFP mRNA expression levels from the CRBR-edited allele (FIG. 4D) were quantified in five sorted GFP-positive MIN6 cells (#8, 10, 13, 14, and 15) by normalizing to mGapdh, while the wild-type (WT) MIN6 control cell line had no detectable EGFP signal (Ct value >36, used 40 for calculation if undetermined). Mouse Ins2 mRNA expression levels (FIG. 4E) were quantified by normalizing to mGapdh first, and then the relative fold change in expression was calculated relative to MIN6 WT cells. Quantification represents n=4 per sorted cell line. Data are represented as mean±SE. All five GFP-positive cell lines were significantly different from the wild-type MIN6 control for both EGFP and mIns2 expression levels, p<0.001.

FIGS. 5A-5E depict CRBR-mediated in vivo EGFP CDS integration in mouse Ins2 gene. FIG. 5A shows a schematic of CRBR AAV vectors used in AAV delivery to Cas9-EGFP mice or wild-type mice. The AAV vector provides the same EGFP CRBR cassette as in the EGFP-2cut donor plasmid but also includes a U6-driven mIns2utr5-sgRNA. Cas9 is expressed in all tissues under the universal promoter CAG in the Cas9-EGFP mice. FIGS. 5B and 5C show two-week-old Cas9-EGFP mice from one litter (four males and five females) were injected with two doses or one dose (40 μL or 20 μL) of AAV8-U6-mINS2utr5sg-EGFP-2cut via r.o. injection with un-injected mice serving as a control. DNA and RNA from pancreas and liver were isolated 30d post-injection. Genomic DNA (FIG. 5B) was tested by 5′ and 3′ junction diagnostic PCRs and by ddPCR quantification of the CRBR integration of EGFP CDS into chromosome 7 (chr7). The percentage of CRBR editing was calculated by normalizing the 5′ junction event to an internal control (mRpp30 on chr19, two copies per pancreatic cell, four copies per hepatocyte). EGFP mRNA expression (FIG. 5C) from the CRBR-edited mIns2 gene was measured by using a forward primer targeting mIns2 5′UTR and a reverse primer (R1 or R2) targeting EGFP to avoid picking up signals from the endogenous EGFP of the Cas9-EGFP mouse strain. The relative fold changes were quantified by normalizing to mActin first and then calculated relative to the no injection control. Quantification represents n=8 (mice with two different dosages of injection showed no dosage effect, therefore, were pooled together for liver and pancreas comparison). Data are represented as mean±SE. FIG. 5D shows eight-week-old Cas9-EGFP mice from two litters (litter^{a} or litter^{b}, gender is indicated in FIGS. 5A-5E) that were injected with 50 μL of AAV-U6-mINS2utr5sg-EGFP-2cut in serotype DJ or 8, or a saline control via tail vein injection. Genomic DNA from pancreas and liver was isolated 35d post-injection. CRBR editing at genome level was tested by 5′ and 3′ junction diagnostic PCRs and by ddPCR quantification of the CRBR integration as in B, n=5. FIG. 5E shows six-month-old C57BL/6J mice from three litters (litter^{a, b or c}, gender is indicated in FIGS. 5A-5E) that were injected with 50 μL of AAV-U6-mINS2utr5sg-EGFP-2cut with or without 50 μL of AAV-nEF-Cas9 in serotype DJ, or saline via tail vein injection. Genomic DNA from pancreas and liver was isolated 35d post-injection. CRBR editing at genome level was tested by 5′ and 3′ junction diagnostic PCRs and by ddPCR quantification of the CRBR integration as in FIG. 5B, n=4. For FIGS. 5B, 5D, and 5E, all primers were designed to flank the junction sites, the same as FIGS. 4A-4E for the MIN6 cell line (solid triangle: 5′, 452 bp; 3′, 690 bp). The hollow triangle marks a nonspecific band recognized by 5′ junction PCR primers. PC, positive control, was genomic DNA from MIN6 cells co-transfected with EGFP-2cut donor and pX459-mINS2utr5sg. Statistically significant differences in CRBR editing efficiency at genome level were seen between pancreas and liver, and between AAV serotypes; *p<0.05, ***p<0.001. Titer of AAV used: AAV8-U6-mINS2utr5sg-EGFP-2cut, 6.15×10¹³GC/mL; AAV-DJ-U6-mINS2utr5sg-EGFP-2cut, 2.92×10¹²GC/mL; AAV-DJ-nEF-Cas9, 3.83×10¹²GC/mL.

FIGS. 6A-6F show CRBR-mediated ex vivo CopGFP CDS integration in human INS gene via plasmid transfection. FIG. 6A depicts a schematic of CRBR-CopGFP-2cut strategy for wild-type hINS genome. The donor plasmid provides a 3′intron1-utr5(in exon2)-CopGFP-SV40 pA cassette that is flanked by Cas9/gRNA target sites in reverse orientation (5′ CCN-20 nt 3′) as identified within the hINS intron 1 (5′ 20 nt-NGG 3′), and a U6-driven hINSin1-sgRNA. Expression of Cas9 from pnEF-Cas9 and hINSin1-sgRNA from the donor leads to the cleavage of the hINS-in1 cut sites that are engineered in the donor to generate the CRBR cassette, and also a targeted DSB at genomic hINS intron 1 between exon 1 and 5′UTR in exon 2. FIG. 6B shows a schematic of CRBR-CopGFP-1cut strategy for wild-type hINS genome. The 1-cut donor plasmid is the same as the 2-cut donor except for removing the 3′ cut site. Expression of Cas9 and hINSin1-sgRNA leads to the cleavage of the hINS-in1 cut site that is engineered in the donor, linearizing the donor, as well as a targeted DSB at genomic hINS intron 1. The 1-cut insert is 4.2 kb, much larger than the 2-cut insert which is only 0.9 kb. In both 1-cut and 2-cut strategies, correct integration of the CRBR cassette will be retained while incorrect integrant is prone to excision; the 5′ junction in the CRBR-edited hINS intron 1 should be spliced out and results in a wild-type 5′UTR for normal translation initiation of CopGFP. FIGS. 6C-6F shows human cadaveric islets (500 IEQs) that were electroporated with 1 μg of pnEF-Cas9, 1 μg of pU6-hINSin1sg-CopGFP-1cut, 1 μg of pU6-hINSin1sg-CopGFP-2cut, or either donor in combination with pnEF-Cas9 using Neon transfection system. Six-day post-transfection, human islets were imaged (FIG. 6C) as live cultures at 10× objective; scale bar, 100 μm. Genomic DNA (FIG. 6D) was harvested for diagnostic PCRs of the 5′, 2cut 3′, and 1cut 3′ junctions. Primers were designed to flank the junction sites (triangle: 5′, 820 bp; 2cut 3′, 722 bp; 1cut 3′, 654 bp). The percentage of CRBR editing (ddPCR quantification of the CRBR integration of CopGFP CDS into chr11) was calculated by normalizing the 5′ junction event to an internal control (hRPP30 on chr10, two copies per cell). CopGFP mRNA expression levels (FIG. 6E) from the CRBR-edited hINS gene [using a forward primer targeting hINS 5′UTR and a reverse primer (R1 or R2) targeting CopGFP] and hINS mRNA expression levels (FIG. 6F) were quantified by normalizing to hActin.

FIGS. 7A-7G show CRBR-mediated ex vivo CopGFP CDS integration in human INS gene via AAV-DJ transduction. FIG. 7A shows a schematic of CRBR AAV vectors used in the CopGFP-2cut and CopGFP-1cut strategies for wild-type hINS genome targeting. FIGS. 7B and 7C show human cadaveric islets (300 IEQs) were infected with AAV-DJ-nEF-Cas9, AAV-DJ-U6-hINSin1sg-CopGFP-1cut, AAV-DJ-U6-hINSin1sg-CopGFP-2cut, or either donor AAV vector in combination with AAV-DJ-nEF-Cas9 at 60,000 MOI. Human islets were imaged (FIG. 7B) 6d and 10d post-infection as live cultures at 10× objective; scale bar, 100 μm. Genomic DNA (FIG. 7C) was harvested 16d post-infection for 5′ junction PCR. Primers were designed to flank the 5′ junction site and amplify a 476 bp fragment. The solid triangle marks a larger fragment that is only present in Cas9+sgRNA CDS donor treatments. Sequencing of this additional fragment revealed it to encode the left ITR and U6-sgRNA regions of the AAV vector. PC, positive control, was genomic DNA from AD293 cells co-transfected with CopGFP-2cut donor and pX459-hINSin1sg. The percentage of CRBR editing (ddPCR quantification of the CRBR integration of CopGFP CDS into chr11) was calculated by normalizing the 5′ junction event to an internal control (hRPP30 on chr10, two copies cell). Resultant genome diagrams show two possible AAV-1cut integrations: expected 5′ junction generates a nascent mRNA with a 17 bp hairpin which will be spliced out; in the case of Cas9/sgRNA cleavage failure, the whole AAV vector integrant will generate a nascent mRNA with the left ITR-U6sg in the intronic region, which can also be spliced out. FIGS. 7D-7F show a second batch of human cadaveric islets (800 IEQs per replicate) that was infected with AAV-DJ-U6-hINSin1sg-CopGFP-1cut or AAV-DJ-U6-hINSin1sg-CopGFP-2cut in combination with AAV-DJ-nEF-Cas9 at 60,000 MOI. Single cell sorting of 1cut or 2cut treated human islets was performed at 11d post-infection. The percentage of GFP positive cell (FIG. 7D) among total cells sorted [alpha (˜25%), beta (˜60%), delta (˜8%), and other cell types within islet cell cluster] were calculated. RNA was harvested from GFP positive and GFP negative sorted cells. mRNA expression of marker genes for pancreatic endocrine cells (FIGS. 7E and 7F) were quantified by normalizing to hActin. Quantification represents n=3 per treatment. Data are represented as mean±SE. Statistical significances were shown as marked: *p<0.05, **p<0.01, ***p<0.001. FIG. 7G shows a third batch of human cadaveric islets that was treated the same as FIGS. 7B and 7C, and RNA was harvested 18d post-infection. CopGFP mRNA expression levels from the CRBR-edited hINS gene were quantified by normalizing to hActin.

### DETAILED DESCRIPTION

As noted above, the present disclosure relates to novel methods for correcting a gene defect, treating a patient having a disease or disorder characterized by a gene defect, and preparing a chimeric antigen receptor T cell, as well as systems, modified cells, and compositions for the same.

A first aspect relates to a method of correcting a gene defect in a cell. The method includes:

providing in a cell having a gene defect (i) a Cas protein or a first nucleic acid molecule encoding the Cas protein, (ii) a guide RNA that is capable of base-pairing with a region of the defective gene between a promoter and a coding sequence thereof, or a second nucleic acid encoding the guide RNA, and (iii) a DNA template including a replacement coding sequence, which encodes a non-defective protein, and a transcription terminator sequence,

wherein upon binding of the guide RNA to the 5′ untranslated region of the defective gene and cleavage of the 5′ untranslated region by the Cas protein, the DNA template is inserted into the genome of the cell via NHEJ repair pathway to allow for expression of the non-defective protein under control of the promoter while simultaneously blocking the expression of the defective gene, thereby correcting the gene defect.

A further aspect relates to a method of treating a patient having a disease or disorder characterized by a gene defect. The method includes:

repairing the gene defect in one or more cell types that express the defective gene product, said repairing including introducing into the one or more cell types (i) a chimeric Clustered Regularly Interspaced Short Palindromic Repeats (CRISPR) associated (Cas) protein or a first nucleic acid molecule encoding the Cas protein, (ii) a guide RNA that is capable of base-pairing with a region of the defective gene between a promoter and a coding sequence thereof, or a second nucleic acid encoding the guide RNA, and (iii) a DNA template including a replacement coding sequence, which encodes a non-defective protein, and a transcription terminator sequence,

wherein upon binding of the guide RNA to the region of the defective gene and cleavage of that region by the Cas protein, the DNA template is inserted into the genome of the cell via non-homologous end-joining (NHEJ) repair pathway to allow for expression of the non-defective protein under control of the promoter while simultaneously blocking the expression of the defective gene, thereby treating the disease or disorder.

Methods and compositions are provided for modifying a target locus, e.g., genomic locus, in a cell. The methods and compositions employ nuclease agents and nuclease agent recognition sites to enhance homologous recombination events of an insert polynucleotide (or DNA template) into the target locus. These methods and compositions are particularly useful for correcting genetic defects. Each of these components is described in further detail below.

The term “recognition site for a nuclease agent” includes a DNA sequence at which a nick or double-strand break is induced by a nuclease agent. The recognition site for a nuclease agent is preferably native. In specific embodiments, the recognition site is native to the cell and is present only once in the genome of the host cell. This will limit the insert polynucleotide to insertion at the one locus. Such a site can then be used to design nuclease agents that will produce a nick or double-strand break at the native recognition site.

The length of the recognition site can vary, and includes, for example, recognition sites that are about 30-36 bp for a zinc finger nuclease (ZFN) pair (i.e., about 15-18 bp for each ZFN), about 36 bp for a Transcription Activator-Like Effector Nuclease (TALEN), or about 20 bp for a CRISPR/Cas9 guide RNA.

Any nuclease agent that induces a nick or double-strand break into a desired recognition site can be used in the methods and compositions disclosed herein. A naturally occurring or native nuclease agent can be employed so long as the nuclease agent induces a nick or double-strand break in a desired recognition site. Alternatively, a modified or engineered nuclease agent can be employed. An “engineered nuclease agent” includes a nuclease that is engineered (modified or derived) from its native form to specifically recognize and induce a nick or double-strand break in the desired recognition site. Thus, an engineered nuclease agent can be derived from a native naturally occurring nuclease agent or it can be artificially created or synthesized. The modification of the nuclease agent can be as little as one amino acid in a protein cleavage agent or one nucleotide in a nucleic acid cleavage agent. In some embodiments, the engineered nuclease induces a nick or double-strand break in a recognition site, wherein the recognition site was not a sequence that would have been recognized by a native (non-engineered or non-modified) nuclease agent. Producing a nick or double-strand break in a recognition site or other DNA can be referred to herein as “cutting” or “cleaving” the recognition site or other DNA.

Active variants and fragments of the exemplified recognition sites are also provided. Such active variants can comprise at least 65%, 70%, 75%, 80%, 85%, 90%, 91%, 92%, 93%, 94%, 95%, 96%, 97%, 98%, 99% or more sequence identity to the given recognition site, wherein the active variants retain biological activity and hence are capable of being recognized and cleaved by a nuclease agent in a sequence-specific manner Assays to measure the double-strand break of a recognition site by a nuclease agent are known in the art (e.g., TaqMan™, qPCR assay, Frendewey et al., Methods in Enzymology, 2010, 476:295-307, which is incorporated by reference herein in its entirety).

In one embodiment, the nuclease agent is a Transcription Activator-Like Effector Nuclease (TALEN). TALENs are a class of sequence-specific nucleases that can be used to make double-strand breaks at specific target sequences in the genome of a prokaryotic or eukaryotic organism. TALENs are created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TALENs can be engineered to recognize specific DNA target sites and thus, used to make double-strand breaks at desired target sequences. See, WO 2010/079430; Morbitzer et al., PNAS, 107:21617-22 (2010); Scholze & Boch, Virulence, 1:428-432 (2010); Christian et al., Genetics, 186:757-761 (2010); Li et al., Nuc. Acids Res., 39:359-72 (2010); and Miller et al., Nature Biotechnology, 29:143-148 (2011); all of which are hereby incorporated by reference in their entirety.

Examples of suitable TALENs, and methods for preparing suitable TALENs, are disclosed, e.g., in U.S. Patent Application No. 2011/0239315 A1, 2011/0269234 A1, 2011/0145940 A1, 2003/0232410 A1, 2005/0208489 A1, 2005/0026157 A1, 2005/0064474 A1, 2006/0188987 A1, and 2006/0063231 A1, all of which are hereby incorporated by reference in their entirety. In various embodiments, TALENs are engineered that cut in or near a target nucleic acid sequence in, e.g., a locus of interest or a genomic locus of interest, wherein the target nucleic acid sequence is at or near a sequence to be modified by a targeting vector. The TALENs suitable for use with the various methods and compositions provided herein include those that are specifically designed to bind at or near target nucleic acid sequences to be modified by targeting vectors as described herein.

In one embodiment, each monomer of the TALEN includes 33-35 TAL repeats that recognize a single base pair via two hypervariable residues. In one embodiment, the nuclease agent is a chimeric protein including a TAL repeat-based DNA binding domain operably linked to an independent nuclease. In one embodiment, the independent nuclease is a FokI endonuclease. In one embodiment, the nuclease agent includes a first TAL-repeat-based DNA binding domain and a second TAL-repeat-based DNA binding domain, wherein each of the first and the second TAL-repeat-based DNA binding domain is operably linked to a Fold nuclease subunit, wherein the first and the second TAL-repeat-based DNA binding domain recognize two contiguous target DNA sequences in each strand of the target DNA sequence separated by a spacer sequence of varying length (12-20 bp), and wherein the Fold nuclease subunits dimerize to create an active nuclease that makes a double strand break at a target sequence.

The nuclease agent employed in the various methods and compositions disclosed herein can further comprise a zinc-finger nuclease (ZFN). In one embodiment, each monomer of the ZFN includes 3 or more zinc finger-based DNA binding domains, wherein each zinc finger-based DNA binding domain binds to a 3 bp subsite. In other embodiments, the ZFN is a chimeric protein including a zinc finger-based DNA binding domain operably linked to an independent nuclease. In one embodiment, the independent endonuclease is a Fold endonuclease. In one embodiment, the nuclease agent includes a first ZFN and a second ZFN, wherein each of the first ZFN and the second ZFN is operably linked to a Fold nuclease subunit, wherein the first and the second ZFN recognize two contiguous target DNA sequences in each strand of the target DNA sequence separated by about 5-7 bp spacer, and wherein the Fold nuclease subunits dimerize to create an active nuclease that makes a double strand break. See, for example, US20060246567; US20080182332; US20020081614; US20030021776; WO/2002/057308A2; US20130123484; US20100291048; WO/2011/017293A2; and Gaj et al. (2013) Trends in Biotechnology, 31(7):397-405, each of which is herein incorporated by reference in its entirety.

The nuclease agent employed in the various methods and compositions preferably includes a CRISPR/Cas system. Such systems can employ a Cas9 nuclease, which in some instances, is codon-optimized for the desired cell type in which it is to be expressed. The system further employs a fused crRNA-tracrRNA construct that functions with the codon-optimized Cas9. This single RNA is often referred to as a guide RNA or gRNA. Within a gRNA, the crRNA portion is identified as the ‘target sequences’ for the given recognition site and the tracrRNA is often referred to as the ‘scaffold’. This system has been shown to function in a variety of eukaryotic and prokaryotic cells.

Briefly, a short DNA fragment containing the target sequence is inserted into a guide RNA expression plasmid. The gRNA expression plasmid includes the target sequence (in some embodiments around 20 nucleotides), a form of the tracrRNA sequence (the scaffold) as well as a suitable promoter that is active in the cell and necessary elements for proper processing in eukaryotic cells. Many of the systems rely on custom, complementary oligos that are annealed to form a double stranded DNA and then cloned into the gRNA expression plasmid. The gRNA expression cassette and the Cas9 expression cassette are then introduced into the cell. See, for example, Mali P et al., Science, 339(6121):823-6 (2013); Jinek M et al., Science, 337(6096):816-21 (2012); Hwang et al., Nat Biotechnol, 31(3):227-9 (2013); Jiang et al., Nat Biotechnol, 31(3):233-9 (2013); and Cong et al., Science, 339(6121):819-23 (2013), each of which is hereby incorporated by reference in its entirety.

The methods and compositions disclosed herein can utilize CRISPR/Cas systems or components of such systems to modify a genome within a cell. CRISPR/Cas systems include transcripts and other elements involved in the expression of, or directing the activity of, Cas genes. A CRISPR/Cas system can be a type I, a type II, or a type III system. The methods and compositions disclosed herein employ CRISPR/Cas systems by utilizing CRISPR complexes (including a guide RNA (gRNA) complexed with a Cas protein) for site-directed cleavage of nucleic acids.

Some CRISPR/Cas systems used in the methods disclosed herein are non-naturally occurring. A “non-naturally occurring” system includes anything indicating the involvement of the hand of man, such as one or more components of the system being altered or mutated from their naturally occurring state, being at least substantially free from at least one other component with which they are naturally associated in nature, or being associated with at least one other component with which they are not naturally associated. For example, some CRISPR/Cas systems employ non-naturally occurring CRISPR complexes including a gRNA and a Cas protein that do not naturally occur together.

Cas proteins generally comprise at least one RNA recognition or binding domain. Such domains can interact with guide RNAs (gRNAs, described in more detail below). Cas proteins can also comprise nuclease domains (e.g., DNase or RNase domains), DNA binding domains, helicase domains, protein-protein interaction domains, dimerization domains, and other domains. A nuclease domain possesses catalytic activity for nucleic acid cleavage. Cleavage includes the breakage of the covalent bonds of a nucleic acid molecule. Cleavage can produce blunt ends or staggered ends, and it can be single-stranded or double-stranded.

Examples of Cas proteins include Cast, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cul966, and homologs or modified versions thereof.

Cas proteins can be from a type II CRISPR/Cas system. For example, the Cas protein can be a Cas9 protein or be derived from a Cas9 protein. Cas9 proteins typically share four key motifs with a conserved architecture. Motifs 1, 2, and 4 are RuvC-like motifs, and motif 3 is an HNH motif. The Cas9 protein can be from, for example, Streptococcus pyogenes, Streptococcus thermophilus, Streptococcus sp., Staphylococcus aureus, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicellulosiruptor bescii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. Additional examples of the Cas9 family members are described in WO 2014/131833, herein incorporated by reference in its entirety. Cas9 protein from S. pyogenes or derived therefrom is a preferred enzyme. Cas9 protein from S. pyogenes is assigned SwissProt accession number Q99ZW2 (SEQ ID NO: 1).

Cas proteins can be wild type proteins (i.e., those that occur in nature), modified Cas proteins (i.e., Cas protein variants), or fragments of wild type or modified Cas proteins. Cas proteins can also be active variants or fragments of wild type or modified Cas proteins. Active variants or fragments can comprise at least 80%, 85%, 90%, 91%, 92%, 93%, 94%, 95%, 96%, 97%, 98%, 99% or more sequence identity to the wild type or modified Cas protein or a portion thereof, wherein the active variants retain the ability to cut at a desired cleavage site and hence retain nick-inducing or double-strand-break-inducing activity. Assays for nick-inducing or double-strand-break-inducing activity are known and generally measure the overall activity and specificity of the Cas protein on DNA substrates containing the cleavage site.

Cas proteins can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein.

Some Cas proteins comprise at least two nuclease domains, such as DNase domains. For example, a Cas9 protein can comprise a RuvC-like nuclease domain and an HNH-like nuclease domain. The RuvC and HNH domains can each cut a different strand of double-stranded DNA to make a double-stranded break in the DNA. See, e.g., Jinek et al., Science, 337:816-821 (2012), hereby incorporated by reference in its entirety.

One or both of the nuclease domains can be deleted or mutated so that they are no longer functional or have reduced nuclease activity. If one of the nuclease domains is deleted or mutated, the resulting Cas protein (e.g., Cas9) can be referred to as a nickase and can generate a single-strand break at a CRISPR RNA recognition sequence within a double-stranded DNA but not a double-strand break (i.e., it can cleave the complementary strand or the non-complementary strand, but not both). If both of the nuclease domains are deleted or mutated, the resulting Cas protein (e.g., Cas9) will have a reduced ability to cleave both strands of a double-stranded DNA. An example of a mutation that converts Cas9 into a nickase is a D10A (aspartate to alanine at position 10 of Cas9) mutation in the RuvC domain of Cas9 from S. pyogenes. Likewise, H939A (histidine to alanine at amino acid position 839) or H840A (histidine to alanine at amino acid position 840) in the HNH domain of Cas9 from S. pyogenes can convert the Cas9 into a nickase. Other examples of mutations that convert Cas9 into a nickase include the corresponding mutations to Cas9 from S. thermophilus. See, e.g., Sapranauskas et al., Nucleic Acids Research, 39:9275-9282 (2011) and WO 2013/141680, each of which is herein incorporated by reference in its entirety. Such mutations can be generated using methods such as site-directed mutagenesis, PCR-mediated mutagenesis, or total gene synthesis. Examples of other mutations creating nickases can be found, for example, in WO/2013/176772A1 and WO/2013/142578A1, each of which is herein incorporated by reference.

Cas proteins can also be fusion proteins. For example, a Cas protein can be fused to a cleavage domain, an epigenetic modification domain, a transcriptional activation domain, or a transcriptional repressor domain. See WO 2014/089290, incorporated herein by reference in its entirety. Cas proteins can also be fused to a heterologous polypeptide providing increased or decreased stability. The fused domain or heterologous polypeptide can be located at the N-terminus, the C-terminus, or internally within the Cas protein.

A Cas protein can be fused to a heterologous polypeptide that provides for subcellular localization. Such heterologous peptides include, for example, a nuclear localization signal (NLS) such as the SV40 NLS for targeting to the nucleus, a mitochondrial localization signal for targeting to the mitochondria, an ER retention signal, and the like. See, e.g., Lange et al., J. Biol. Chem., 282:5101-5105 (2007), which is hereby incorporated by reference in its entirety. Such subcellular localization signals can be located at the N-terminus, the C-terminus, or anywhere within the Cas protein. An NLS can comprise a stretch of basic amino acids, and can be a monopartite sequence or a bipartite sequence.

Cas proteins can also be linked to a cell-penetrating domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, or a polyarginine peptide sequence. See, for example, WO 2014/089290, herein incorporated by reference in its entirety. The cell-penetrating domain can be located at the N-terminus, the C-terminus, or anywhere within the Cas protein.

Cas proteins can also comprise a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreen1), yellow fluorescent proteins (e.g., YFP, eYFP, Citrin, Venus, YPet, PhiYFP, ZsYellow1), blue fluorescent proteins (e.g. eBFP, eBFP2, Azurite, mKalama1, GFPuv, Sapphire, T-sapphire), cyan fluorescent proteins (e.g. eCFP, Cerulean, CyPet, AmCyan1, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, Jred), orange fluorescent proteins (mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, hemagglutinin (HA), nus, Softag 1, Softag 3, Strep, SBP, Glu-Glu, HSV, KT3, S, 51, T7, V5, VSV-G, histidine (His), biotin carboxyl carrier protein (BCCP), and calmodulin.

Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Optionally, the nucleic acid encoding the Cas protein can be codon optimized for efficient translation into protein in a particular cell or organism.

Nucleic acids encoding Cas proteins can be stably integrated in the genome of the cell and operably linked to a promoter active in the cell. Alternatively, nucleic acids encoding Cas proteins can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. Promoters that can be used in an expression construct include, for example, promoters active in a pluripotent rat, eukaryotic, mammalian, non-human mammalian, human, rodent, mouse, or hamster cell. Examples of other promoters are described elsewhere herein.

A “guide RNA” or “gRNA” includes an RNA molecule that binds to a Cas protein and targets the Cas protein to a specific location within a target DNA. Guide RNAs can comprise two segments: a “DNA-targeting segment” and a “protein-binding segment.” “Segment” includes a segment, section, or region of a molecule, such as a contiguous stretch of nucleotides in an RNA. Some gRNAs comprise two separate RNA molecules: an “activator-RNA” and a “targeter-RNA.” Other gRNAs are a single RNA molecule (single RNA polynucleotide), which can also be called a “single-molecule gRNA,” a “single-guide RNA,” or an “sgRNA.” See, e.g., WO/2013/176772A1, WO/2014/065596A1, WO/2014/089290A1, WO/2014/093622A2, WO/2014/099750A2, WO/2013142578A1, and WO 2014/131833A1, each of which is herein incorporated by reference. The terms “guide RNA” and “gRNA” include both double-molecule gRNAs and single-molecule gRNAs.

An exemplary two-molecule gRNA includes a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA” or “scaffold”) molecule. A crRNA includes both the DNA-targeting segment (single-stranded) of the gRNA and a stretch of nucleotides that forms one half of the dsRNA duplex of the protein-binding segment of the gRNA.

A corresponding tracrRNA (activator-RNA) includes a stretch of nucleotides that forms the other half of the dsRNA duplex of the protein-binding segment of the gRNA. A stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA.

The crRNA and the corresponding tracrRNA hybridize to form a gRNA. The crRNA additionally provides the single-stranded DNA-targeting segment that hybridizes to a CRISPR RNA recognition sequence. If used for modification within a cell, the exact sequence of a given crRNA or tracrRNA molecule can be designed to be specific to the species in which the RNA molecules will be used. See, for example, Mali et al., Science, 339:823-826 (2013); Jinek et al. Science, 337:816-821 (2012); Hwang et al., Nat. Biotechnol., 31:227-229 (2013); Jiang et al. Nat. Biotechnol., 31:233-239 (2013); and Cong et al. Science, 339:819-823 (2013), each of which is herein incorporated by reference.

The DNA-targeting segment (crRNA) of a given gRNA includes a nucleotide sequence that is complementary to a sequence in a target DNA. The DNA-targeting segment of a gRNA interacts with a target DNA in a sequence-specific manner via hybridization (i.e., base pairing). As such, the nucleotide sequence of the DNA-targeting segment may vary and determines the location within the target DNA with which the gRNA and the target DNA will interact. The DNA-targeting segment of a subject gRNA can be modified to hybridize to any desired sequence within a target DNA. Naturally occurring crRNAs differ depending on the Cas9 system and organism but often contain a targeting segment of between 21 to 72 nucleotides length, flanked by two direct repeats (DR) of a length of between 21 to 46 nucleotides (see, e.g., WO2014/131833). In the case of S. pyogenes, the DRs are 36 nucleotides long and the targeting segment is 30 nucleotides long. The 3′ located DR is complementary to and hybridizes with the corresponding tracrRNA, which in turn binds to the Cas9 protein.

The DNA-targeting segment can have a length of from about 12 nucleotides to about 100 nucleotides. For example, the DNA-targeting segment can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. Alternatively, the DNA-targeting segment can have a length of from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt.

The nucleotide sequence of the DNA-targeting segment that is complementary to a nucleotide sequence (CRISPR RNA recognition sequence) of the target DNA can have a length at least about 12 nt. For example, the DNA-targeting sequence (i.e., the sequence within the DNA-targeting segment that is complementary to a CRISPR RNA recognition sequence within the target DNA) can have a length at least about 12 nt, at least about 15 nt, at least about 18 nt, at least about 19 nt, at least about 20 nt, at least about 25 nt, at least about 30 nt, at least about 35 nt, or at least about 40 nt. Alternatively, the DNA-targeting sequence can have a length of from about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some cases, the DNA-targeting sequence can have a length of at about 20 nt.

TracrRNAs can be in any form (e.g., full-length tracrRNAs or active partial tracrRNAs) and of varying lengths. They can include primary transcripts or processed forms. For example, tracrRNAs (as part of a single-guide RNA or as a separate molecule as part of a two-molecule gRNA) may comprise or consist of all or a portion of a wild-type tracrRNA sequence (e.g., about or more than about 20, 26, 32, 45, 48, 54, 63, 67, 85, or more nucleotides of a wild-type tracrRNA sequence). Examples of wild-type tracrRNA sequences from S. pyogenes include 171-nucleotide, 89-nucleotide, 75-nucleotide, and 65-nucleotide versions. See, for example, Deltcheva et al., Nature, 471:602-607 (2011); WO 2014/093661, each of which is incorporated herein by reference in their entirety. Examples of tracrRNAs within single-guide RNAs (sgRNAs) include the tracrRNA segments found within +48, +54, +67, and +85 versions of sgRNAs, where “+n” indicates that up to the +n nucleotide of wild-type tracrRNA is included in the sgRNA. See U.S. Pat. No. 8,697,359, incorporated herein by reference in its entirety.

The percent complementarity between the DNA-targeting sequence and the CRISPR RNA recognition sequence within the target DNA can be at least 60% (e.g., at least 65%, at least 70%, at least 75%, at least 80%, at least 85%, at least 90%, at least 95%, at least 97%, at least 98%, at least 99%, or 100%). The percent complementarity between the DNA-targeting sequence and the CRISPR RNA recognition sequence within the target DNA can be at least 60% over about 20 contiguous nucleotides. As an example, the percent complementarity between the DNA-targeting sequence and the CRISPR RNA recognition sequence within the target DNA is 100% over the 14 contiguous nucleotides at the 5′ end of the CRISPR RNA recognition sequence within the complementary strand of the target DNA and as low as 0% over the remainder. In such a case, the DNA-targeting sequence can be considered to be 14 nucleotides in length. As another example, the percent complementarity between the DNA-targeting sequence and the CRISPR RNA recognition sequence within the target DNA is 100% over the seven contiguous nucleotides at the 5′ end of the CRISPR RNA recognition sequence within the complementary strand of the target DNA and as low as 0% over the remainder. In such a case, the DNA-targeting sequence can be considered to be 7 nucleotides in length.

The protein-binding segment of a gRNA can comprise two stretches of nucleotides that are complementary to one another. The complementary nucleotides of the protein-binding segment hybridize to form a double-stranded RNA duplex (dsRNA). The protein-binding segment of a subject gRNA interacts with a Cas protein, and the gRNA directs the bound Cas protein to a specific nucleotide sequence within target DNA via the DNA-targeting segment.

Guide RNAs can include modifications or sequences that provide for additional desirable features (e.g., modified or regulated stability; subcellular targeting; tracking with a fluorescent label; a binding site for a protein or protein complex; and the like). Examples of such modifications include, for example, a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof.

Guide RNAs can be provided in any form. For example, the gRNA can be provided in the form of RNA, either as two molecules (separate crRNA and tracrRNA) or as one molecule (sgRNA), and optionally in the form of a complex with a Cas protein. The gRNA can also be provided in the form of DNA encoding the gRNA. The DNA encoding the gRNA can encode a single RNA molecule (sgRNA) or separate RNA molecules (e.g., separate crRNA and tracrRNA). In the latter case, the DNA encoding the gRNA can be provided as separate DNA molecules encoding the crRNA and tracrRNA, respectively.

DNAs encoding gRNAs can be stably integrated in the genome of the cell and operably linked to a promoter active in the cell. Alternatively, DNAs encoding gRNAs can be operably linked to a promoter in an expression construct. Such promoters can be active, for example, in a pluripotent rat, eukaryotic, mammalian, non-human mammalian, human, rodent, mouse, or hamster cell. In some instances, the promoter is an RNA polymerase III promoter, such as a human U6 promoter, a rat U6 polymerase III promoter, or a mouse U6 polymerase III promoter. Examples of other promoters are described elsewhere herein.

Alternatively, gRNAs can be prepared by various other methods. For example, gRNAs can be prepared by in vitro transcription using, for example, T7 RNA polymerase (see, for example, WO 2014/089290 and WO 2014/065596, which are hereby incorporated by reference in their entirety). Guide RNAs can also be a synthetically produced molecule prepared by chemical synthesis.

Exemplary gRNA are identified in the accompanying Examples.

The term “CRISPR RNA recognition sequence” includes nucleic acid sequences present in a target DNA to which a DNA-targeting segment of a gRNA will bind, provided sufficient conditions for binding exist. For example, CRISPR RNA recognition sequences include sequences to which a guide RNA is designed to have complementarity, where hybridization between a CRISPR RNA recognition sequence and a DNA targeting sequence promotes the formation of a CRISPR complex. Full complementarity is not necessarily required, provided there is sufficient complementarity to cause hybridization and promote formation of a CRISPR complex. CRISPR RNA recognition sequences also include cleavage sites for Cas proteins, described in more detail below. A CRISPR RNA recognition sequence can comprise any polynucleotide, which can be located, for example, in the nucleus or cytoplasm of a cell or within an organelle of a cell, such as a mitochondrion or chloroplast.

The CRISPR RNA recognition sequence within a target DNA can be targeted by (i.e., be bound by, or hybridize with, or be complementary to) a Cas protein or a gRNA. Suitable DNA/RNA binding conditions include physiological conditions normally present in a cell. Other suitable DNA/RNA binding conditions (e.g., conditions in a cell-free system) are known in the art (see, e.g., Molecular Cloning: A Laboratory Manual, 3rd Ed. (Sambrook et al., Harbor Laboratory Press 2001)). The strand of the target DNA that is complementary to and hybridizes with the Cas protein or gRNA can be called the “complementary strand,” and the strand of the target DNA that is complementary to the “complementary strand” (and is therefore not complementary to the Cas protein or gRNA) can be called “noncomplementary strand” or “template strand.”

The Cas protein can cleave the nucleic acid at a site within or outside of the nucleic acid sequence present in the target DNA to which the DNA-targeting segment of a gRNA will bind. The “cleavage site” includes the position of a nucleic acid at which a Cas protein produces a single-strand break or a double-strand break. For example, formation of a CRISPR complex (including a gRNA hybridized to a CRISPR RNA recognition sequence and complexed with a Cas protein) can result in cleavage of one or both strands in or near (e.g., within 1, 2, 3, 4, 5, 6, 7, 8, 9, 10, 20, 50, or more base pairs from) the nucleic acid sequence present in a target DNA to which a DNA-targeting segment of a gRNA will bind. If the cleavage site is outside of the nucleic acid sequence to which the DNA-targeting segment of the gRNA will bind, the cleavage site is still considered to be within the “CRISPR RNA recognition sequence.” The cleavage site can be on only one strand or on both strands of a nucleic acid. Cleavage sites can be at the same position on both strands of the nucleic acid (producing blunt ends) or can be at different sites on each strand (producing staggered ends). Staggered ends can be produced, for example, by using two Cas proteins, each of which produces a single-strand break at a different cleavage site on each strand, thereby producing a double-strand break. For example, a first nickase can create a single-strand break on the first strand of double-stranded DNA (dsDNA), and a second nickase can create a single-strand break on the second strand of dsDNA such that overhanging sequences are created. In some cases, the CRISPR RNA recognition sequence of the nickase on the first strand is separated from the CRISPR RNA recognition sequence of the nickase on the second strand by at least 2, 3, 4, 5, 6, 7, 8, 9, 10, 15, 20, 25, 30, 40, 50, 75, 100, 250, 500, or 1,000 base pairs.

Site-specific cleavage of target DNA by Cas9 can occur at locations determined by both (i) base-pairing complementarity between the gRNA and the target DNA and (ii) a short motif, called the protospacer adjacent motif (PAM), in the target DNA. The PAM can flank the CRISPR RNA recognition sequence. Optionally, the CRISPR RNA recognition sequence can be flanked by the PAM. For example, the cleavage site of Cas9 can be about 1 to about 10 or about 2 to about 5 base pairs (e.g., 3 base pairs) upstream or downstream of the PAM sequence. In some cases (e.g., when Cas9 from S. pyogenes or a closely related Cas9 is used), the PAM sequence of the non-complementary strand can be 5′-N₁GG-3′, where N₁ is any DNA nucleotide and is immediately 3′ of the CRISPR RNA recognition sequence of the non-complementary strand of the target DNA. As such, the PAM sequence of the complementary strand would be 5′-CCN₂-3′, where N₂ is any DNA nucleotide and is immediately 5′ of the CRISPR RNA recognition sequence of the complementary strand of the target DNA. In some such cases, N₁ and N₂ can be complementary and the N₁-N₂base pair can be any base pair (e.g., N₁=C and N₂=G; N₁=G and N₂=C; N₁=A and N₂=T, N₁=T, and N₂=A).

Examples of CRISPR RNA recognition sequences include a DNA sequence complementary to the DNA-targeting segment of a gRNA, or such a DNA sequence in addition to a PAM sequence. For example, the target motif can be a 20-nucleotide DNA sequence immediately preceding an NGG motif recognized by a Cas protein (see, for example, WO 2014/165825, which is hereby incorporated by reference in its entirety). The guanine at the 5′ end can facilitate transcription by RNA polymerase in cells. Other examples of CRISPR RNA recognition sequences can include two guanine nucleotides at the 5′ end (e.g., GGN₂₀NGG; SEQ ID NO: 2) to facilitate efficient transcription by T7 polymerase in vitro. See, for example, WO 2014/065596, which is hereby incorporated by reference in its entirety.

The CRISPR RNA recognition sequence can be any nucleic acid sequence endogenous to a cell. The CRISPR RNA recognition sequence is preferably located upstream of the first exon in a native defective gene (to be corrected), and more preferably is located downstream of the native promoter sequence but upstream of the first exon. In one embodiment, the target sequence is immediately flanked by a Protospacer Adjacent Motif (PAM) sequence. In one embodiment, the gRNA includes a third nucleic acid sequence encoding a CRISPR RNA (crRNA) and a trans-activating CRISPR RNA (tracrRNA).

Active variants and fragments of nuclease agents (i.e. an engineered nuclease agent) are also provided. Such active variants can comprise at least 65%, 70%, 75%, 80%, 85%, 90%, 91%, 92%, 93%, 94%, 95%, 96%, 97%, 98%, 99% or more sequence identity to the native nuclease agent, wherein the active variants retain the ability to cut at a desired recognition site and hence retain nick or double-strand-break-inducing activity. For example, any of the nuclease agents described herein can be modified from a native endonuclease sequence and designed to recognize and induce a nick or double-strand break at a recognition site that was not recognized by the native nuclease agent. Thus, in some embodiments, the engineered nuclease has a specificity to induce a nick or double-strand break at a recognition site that is different from the corresponding native nuclease agent recognition site. Assays for nick or double-strand-break-inducing activity are known and generally measure the overall activity and specificity of the endonuclease on DNA substrates containing the recognition site.

The nuclease agent may be introduced into the cell by any means known in the art. The polypeptide encoding the nuclease agent may be directly introduced into the cell. Alternatively, a polynucleotide encoding the nuclease agent can be introduced into the cell. When a polynucleotide encoding the nuclease agent is introduced into the cell, the nuclease agent can be transiently, conditionally or constitutive expressed within the cell. Thus, the polynucleotide encoding the nuclease agent can be contained in an expression cassette and be operably linked to a conditional promoter, an inducible promoter, a constitutive promoter, or a tissue-specific promoter. Such promoters of interest are discussed in further detail elsewhere herein. Alternatively, the nuclease agent is introduced into the cell as an mRNA encoding a nuclease agent.

In specific embodiments, the polynucleotide encoding the nuclease agent is stably integrated in the genome of the cell and operably linked to a promoter active in the cell. In other embodiments, the polynucleotide encoding the nuclease agent is in the same targeting vector including the insert polynucleotide, while in other instances the polynucleotide encoding the nuclease agent is in a vector or a plasmid that is separate from the targeting vector including the insert polynucleotide.

When the nuclease agent is provided to the cell through the introduction of a polynucleotide encoding the nuclease agent, such a polynucleotide encoding a nuclease agent can be modified to substitute codons having a higher frequency of usage in the cell of interest, as compared to the naturally occurring polynucleotide sequence encoding the nuclease agent. For example the polynucleotide encoding the nuclease agent can be modified to substitute codons having a higher frequency of usage in a given prokaryotic or eukaryotic cell of interest, including a bacterial cell, a yeast cell, a human cell, a non-human cell, a mammalian cell, a rodent cell, a mouse cell, a rat cell or any other host cell of interest, as compared to the naturally occurring polynucleotide sequence.

The various methods and compositions provided herein employ the nuclease agents and their corresponding recognition sites in combination with selection markers. In certain embodiments, the position of the recognition site in the polynucleotide encoding the selection marker allows for an efficient method by which to identify integration events at the target locus. Moreover, various methods are provided herein wherein alternating selection markers having the nuclease recognition site are employed to improve the efficiency and efficacy through which multiple polynucleotides of interest are integrated within a given targeted locus.

Various selection markers can be used in the methods and compositions disclosed herein. Such selection markers can, for example, impart resistance to an antibiotic such as G418, hygromycin, blastocidin, neomycin, or puromycin. Such selection markers include neomycin phosphotransferase (neo^{r}), hygromycin b phosphotransferase (hyg^{r}), puromycin-n-acetyltransferase (puro^{r}), and blasticidin s deaminase (bsr^{r}). In still other embodiments, the selection marker is operably linked to an inducible promoter and the expression of the selection marker is toxic to the cell. Non-limiting examples of such selection markers include xanthine/guanine phosphoribosyl transferase (gpt), hypoxanthine-guanine phosphoribosyltransferase (HGPRT) or herpes simplex virus thymidine kinase (HSV-TK).

The polynucleotide encoding the selection markers are operably linked to a promoter active in the cell. Such expression cassettes and their various regulatory components are discussed in further detailed elsewhere herein.

Various methods and compositions are provided, which allow for the integration of at least one insert polynucleotide at a target locus. The term “target locus” includes any segment or region of DNA that one desires to integrate an insert polynucleotide. In one embodiment, the target locus is preferably located upstream of the first exon in a native defective gene (to be corrected), and more preferably is located downstream of the native promoter sequence but upstream of the first exon.

Non-limiting examples of the target locus include a genomic locus associated with a defective gene that encodes a defective protein (e.g., expressed in a B cell, an immature B cell, a mature B cell), or a T cell receptor loci, including for example a T cell receptor alpha locus. Such locus can be from a bird (e.g., a chicken), a non-human mammal, a rodent, a human, a rat, a mouse, a hamster, a rabbit, a pig, a bovine, a deer, a sheep, a goat, a cat, a dog, a ferret, a primate (e.g., marmoset, rhesus monkey), domesticated mammal or an agricultural mammal or any other organism of interest or a combination thereof.

As outlined above, the methods and compositions provided herein take advantage of nuclease agents. Such methods employ the nick or double-strand break at the recognition site in combination with homologous recombination to thereby target the integration of an insert polynucleotide into the target locus. “Homologous recombination” is used conventionally to include the exchange of DNA fragments between two DNA molecules at cross-over sites within the regions of homology.

The term “insert polynucleotide” is used herein interchangeably with “DNA Template”, and includes a segment of DNA that one desires to integrate at the target locus. In one embodiment, the insert polynucleotide includes one or more polynucleotides of interest, preferably a polynucleotide that encodes a wildtype polypeptide or a polypeptide that is modified in one or more respects but otherwise overcomes the genetic defects caused by the defective protein or polypeptide of the defective gene.

In preferred embodiments, the insert polynucleotide, or DNA Template, includes or consists of a complete open reading frame that encodes a wildtype polypeptide or a polypeptide that is modified in one or more respects but otherwise overcomes the genetic defects caused by the defective protein or polypeptide of the defective gene, and a transcription/translation termination signal. By insertion of the insert polynucleotide, or DNA Template, into the region located downstream of the native promoter sequence but upstream of the first exon in the native gene, it is possible to replace a defective coding sequence with the DNA template such that the encoded wildtype or modified polypeptide is expressed but, due to the transcription/translation termination signal, the defective coding sequence is not. In one embodiment, the non-defective protein is a wild-type variant or a modified variant having improved activity relative to wild-type.

In other embodiments, the insert polynucleotide can comprise one or more expression cassettes. A given expression cassette can comprise a polynucleotide of interest, a polynucleotide encoding a selection marker and/or a reporter gene along with the various regulatory components that influence expression. Non-limiting examples of polynucleotides of interest, selection markers, and reporter genes (e.g., eGFP) that can be included within the insert polynucleotide are discussed in detail elsewhere herein.

In specific embodiments, the insert polynucleotide can comprise a genomic nucleic acid. In one embodiment, the genomic nucleic acid is derived from a mouse, a human, a rodent, a non-human, a rat, a hamster, a rabbit, a pig, a bovine, a deer, a sheep, a goat, a chicken, a cat, a dog, a ferret, a primate (e.g., marmoset, rhesus monkey), domesticated mammal or an agricultural mammal or any other organism of interest or a combination thereof.

The insert polynucleotide can be from about 5 kb to about 200 kb, from about 5 kb to about 10 kb, from about 10 kb to about 20 kb, from about 20 kb to about 30 kb, from about 30 kb to about 40 kb, from about 40 kb to about 50 kb, from about 60 kb to about 70 kb, from about 80 kb to about 90 kb, from about 90 kb to about 100 kb, from about 100 kb to about 110 kb, from about 120 kb to about 130 kb, from about 130 kb to about 140 kb, from about 140 kb to about 150 kb, from about 150 kb to about 160 kb, from about 160 kb to about 170 kb, from about 170 kb to about 180 kb, from about 180 kb to about 190 kb, or from about 190 kb to about 200 kb.

In specific embodiments, the insert polynucleotide includes a nucleic acid flanked with site-specific recombination target sequences. It is recognized that while the entire insert polynucleotide can be flanked by such site-specific recombination target sequence, any region or individual polynucleotide of interest within the insert polynucleotide can also be flanked by such sites. The term “recombination site” includes a nucleotide sequence that is recognized by a site-specific recombinase and that can serve as a substrate for a recombination event. The term “site-specific recombinase” includes a group of enzymes that can facilitate recombination between recombination sites where the two recombination sites are physically separated within a single nucleic acid molecule or on separate nucleic acid molecules. Examples of site-specific recombinases include, but are not limited to, Cre, Flp, and Dre recombinases. The site-specific recombinase can be introduced into the cell by any means, including by introducing the recombinase polypeptide into the cell or by introducing a polynucleotide encoding the site-specific recombinase into the host cell. The polynucleotide encoding the site-specific recombinase can be located within the insert polynucleotide or within a separate polynucleotide. The site-specific recombinase can be operably linked to a promoter active in the cell including, for example, an inducible promoter, a promoter that is endogenous to the cell, a promoter that is heterologous to the cell, a cell-specific promoter, a tissue-specific promoter, or a developmental stage-specific promoter. Site-specific recombination target sequences which can flank the insert polynucleotide or any polynucleotide of interest in the insert polynucleotide can include, but are not limited to, loxP, lox511, lox2272, lox66, lox71, loxM2, lox5171, FRT, FRT11, FRT71, attp, att, FRT, rox, and a combination thereof.

In other embodiments, the site-specific recombination sites flank a polynucleotide encoding a selection marker and/or a reporter gene contained within the insert polynucleotide. In such instances following integration of the insert polynucleotide at the targeted locus the sequences between the site-specific recombination sites can be removed.

In one embodiment, the insert polynucleotide includes a polynucleotide encoding a selection marker. Such selection markers include, but are not limited, to neomycin phosphotransferase (neo^{r}), hygromycin B phosphotransferase (hyg^{r}), puromycin-N-acetyltransferase (puro^{r}), blasticidin S deaminase (bsr^{r}), xanthine/guanine phosphoribosyl transferase (gpt), or herpes simplex virus thymidine kinase (HSV-k), or a combination thereof. In one embodiment, the polynucleotide encoding the selection marker is operably linked to a promoter active in the cell. When serially tiling polynucleotides of interest into a targeted locus (i.e., a genomic locus), the selection marker can comprise a recognition site for a nuclease agent, as outlined above. In one embodiment, the polynucleotide encoding the selection marker is flanked with a site-specific recombination target sequences.

The insert polynucleotide can further comprise a reporter gene operably linked to a promoter, wherein the reporter gene encodes a reporter protein selected from the group consisting of LacZ, mPlum, mCherry, tdTomato, mStrawberry, J-Red, DsRed, mOrange, mKO, mCitrine, Venus, YPet, enhanced yellow fluorescent protein (EYFP), Emerald, enhanced green fluorescent protein (EGFP), CyPet, cyan fluorescent protein (CFP), Cerulean, T-Sapphire, luciferase, alkaline phosphatase, and a combination thereof. Such reporter genes can be operably linked to a promoter active in the cell. Such promoters can be an inducible promoter, a promoter that is endogenous to the reporter gene or the cell, a promoter that is heterologous to the reporter gene or to the cell, a cell-specific promoter, a tissue-specific promoter manner or a developmental stage-specific promoter.

Targeting vectors are employed to introduce the insert polynucleotide into the targeted locus. The targeting vector includes the insert polynucleotide and further includes an upstream and a downstream homology arm, which flank the insert polynucleotide. The homology arms, which flank the insert polynucleotide, correspond to regions within the targeted locus. For ease of reference, the corresponding regions within the targeted locus are referred to herein as “target sites”. Thus, in one example, a targeting vector can comprise a first insert polynucleotide flanked by a first and a second homology arm corresponding to a first and a second target site located in sufficient proximity to the first recognition site within the polynucleotide encoding the selection marker. As such, the targeting vector thereby aids in the integration of the insert polynucleotide into the targeted locus through a homologous recombination event that occurs between the homology arms and the corresponding target sites, for example, within the genome of the cell.

A homology arm of the targeting vector can be of any length that is sufficient to promote a homologous recombination event with a corresponding target site, including for example, 50-100 bases, 100-1000 bases or at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 100-200, or 200-300 kilobases in length or greater.

The target sites within the targeted locus that correspond to the upstream and downstream homology arms of the targeting vector are located in “sufficient proximity to the recognition site”. The upstream and downstream homology arms of a targeting vector are “located in sufficient proximity” to a recognition site where the distance is such as to promote the occurrence of a homologous recombination event between the target sites and the homology arms upon a nick or double-strand break at the recognition site. Thus, in specific embodiments, the target sites corresponding to the upstream and/or downstream homology arm of the targeting vector are within at least 1 nucleotide of a given recognition site, are within about 10 nucleotides to about 100 nucleotides, about 100 nucleotides to about 500 nucleotides, about 500 nucleotides to about 1000 nucleotides of a given recognition site. In specific embodiments, the recognition site is immediately adjacent to at least one or both of the target sites.

A homology arm and a target site “correspond” or are “corresponding” to one another when the two regions share a sufficient level of sequence identity to one another to act as substrates for a homologous recombination reaction. By “homology” is meant DNA sequences that are either identical or share sequence identity to a corresponding sequence. The sequence identity between a given target site and the corresponding homology arm found on the targeting vector can be any degree of sequence identity that allows for homologous recombination to occur. For example, the amount of sequence identity shared by the homology arm of the targeting vector (or a fragment thereof) and the target site (or a fragment thereof) can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination. Moreover, a corresponding region of homology between the homology arm and the corresponding target site can be of any length that is sufficient to promote homologous recombination at the cleaved recognition site. For example, a given homology arm and/or corresponding target site can comprise corresponding regions of homology that are at least about 25-50 bases, 50-100 bases, 100-1000 bases, or more than 1 kilobase in length such that the homology arm has sufficient homology to undergo homologous recombination with the corresponding target sites within the genome of the cell.

The homology arms of the targeting vector are therefore designed to correspond to a target site with the targeted locus. Thus, the homology arms can correspond to a locus that is native to the cell. Thus, in specific embodiments, the homology arms of the targeting vector correspond to a locus that is native to a human or a non-human animal such as a bird (e.g., chicken), a non-human mammal, a rodent, a rat, a mouse, a hamster a rabbit, a pig, a bovine, a deer, a sheep, a goat, a cat, a dog, a ferret, a non-human primate (e.g., marmoset, rhesus monkey), domesticated mammal or an agricultural mammal or any other organism of interest.

Methods and compositions are provided for modifying one or more target loci of interest in a cell utilizing a CRISPR/Cas system as described elsewhere herein. For the CRISPR/Cas system, the terms “target site” or “target sequence” can be used interchangeably and include nucleic acid sequences present in a target DNA to which a DNA-targeting segment of a guide RNA (gRNA) will bind, provided sufficient conditions for binding exist. For example, the target site (or target sequence) within a target DNA is targeted by (or is bound by, or hybridizes with, or is complementary to) the Cas nuclease or gRNA. Suitable DNA/RNA binding conditions include physiological conditions normally present in a cell. Other suitable DNA/RNA binding conditions (e.g., conditions in a cell-free system) are known in the art (see, e.g., Molecular Cloning: A Laboratory Manual, 3rd Ed. (Sambrook et al., Harbor Laboratory Press 2001), which is hereby incorporated by reference in its entirety). The strand of the target DNA that is complementary to and hybridizes with the Cas protein or gRNA is referred to as the “complementary strand” and the strand of the target DNA that is complementary to the “complementary strand” (and is therefore not complementary to the Cas protein or gRNA) is referred to as the “noncomplementary strand” or “template strand.”

The Cas protein may cleave the nucleic acid at a site within the target sequence or outside of the target sequence. The “cleavage site” includes the position of a nucleic acid wherein a Cas protein produces a single-strand break or a double-strand break. In one embodiment, the Cas protein is a Cas9 protein. Sticky ends can be produced by using two Cas9 protein which produce a single-strand break at cleavage sites on each strand. Site-specific cleavage of target DNA by Cas9 can occur at locations determined by both (i) base-pairing complementarity between the guide RNA and the target DNA; and (ii) a short motif, referred to as the protospacer adjacent motif (PAM), in the target DNA. For example, the cleavage site of Cas9 can be about 1 to about 10 or about 2 to about 5 base pairs (e.g., 3 base pairs) upstream of the PAM sequence. In some embodiments (e.g., when Cas9 from S. pyogenes, or a closely related Cas9, is used), the PAM sequence of the non-complementary strand can be 5′-XGG-3′, where X is any DNA nucleotide and X is immediately 3′ of the target sequence of the non-complementary strand of the target DNA. As such, the PAM sequence of the complementary strand would be 5′-CCY-3′, where Y is any DNA nucleotide and Y is immediately 5′ of the target sequence of the complementary strand of the target DNA. In some such embodiments, X and Y can be complementary and the X-Y base pair can be any basepair (e.g., X=C and Y=G; X=G and Y=C; X=A and Y=T, X=T and Y=A). In one embodiment, the Cas9 protein is selected from Streptococcus pyogenes Cas9 and Streptococcus aureus Cas9.

The methods include (a) providing a cell comprising a defective gene; (b) introducing into the cell: (i) a CRISPR associated (Cas) protein or a first nucleic acid molecule encoding the Cas protein, (ii) a guide RNA that is capable of base-pairing with a region of the defective gene between a promoter and a coding sequence thereof, or a second nucleic acid encoding the guide RNA, and (iii) a DNA template (insert polynucleotide) including a replacement coding sequence, which encodes a non-defective protein, and a transcription terminator sequence; and, (c) identifying at least one cell including the insert polynucleotide integrated at the target locus.

In one embodiment, the providing or repairing is carried out by introducing into the cell one or more non-viral delivery vehicles including the Cas protein or mRNA encoding the Cas protein, the guide RNA, and the DNA template. In one embodiment, the non-viral delivery vehicle includes a lipid-like nanoparticle, inorganic nanoparticle, cell-penetrating peptide, DNA nanoclew, cationic nanocarrier, zeolitic imidazole framework, zwitterionic amino-lipid nanoparticles, or antibody tissue-targeting. In one embodiment, the guide RNA includes one or more modified bases or a modified backbone.

In one embodiment, the eukaryotic cell is a mammalian cell or a non-human mammalian cell. In one embodiment, the mammalian cell is a fibroblast cell. In one embodiment, the mammalian cell is a human fibroblast cell. In one embodiment, the mammalian cell is a human adult stem cell. In one embodiment, the mammalian cell is a developmentally restricted progenitor cell. In one embodiment, the mammalian cell is a developmentally restricted human progenitor cell.

In one embodiment, the eukaryotic cell is a pluripotent cell. In one embodiment, the pluripotent cell is a hematopoietic stem cell or a neuronal stem cell. In one embodiment, the pluripotent cell is a human induced pluripotent stem (iPS) cell. In one embodiment, the pluripotent cell is a non-human ES cell or a human ES cell.

In one embodiment, the eukaryotic cell is a zygote.

In one embodiment, the first, second, or third insert nucleic acid includes a genomic region of the human T cell receptor alpha locus. In one embodiment, the genomic region includes at least one variable region gene segment and/or a joining region gene segment of the human T cell receptor alpha locus.

Exemplary methods are reported in the accompanying examples.

The polynucleotide of interest within the insert polynucleotide when integrated at the target locus can introduce one or more genetic modifications into the cell. As indicated above, the genetic modification comprises or consists of a complete open reading frame that encodes a wildtype polypeptide or a polypeptide that is modified in one or more respects but otherwise overcomes the genetic defects caused by the defective protein or polypeptide of the defective gene, and a transcription/translation termination signal. By insertion of the insert polynucleotide, or DNA Template, into the region located downstream of the native promoter sequence but upstream of the first exon in the native gene, it is possible to replace a defective coding sequence with the DNA template such that the encoded wildtype or modified polypeptide is expressed but, due to the transcription/translation termination signal, the defective, native coding sequence is not.

The polynucleotide of interest within the insert polynucleotide and/or integrated at the target locus can comprise a sequence that is native or homologous to the cell it is introduced into; the polynucleotide of interest can be heterologous to the cell it is introduced to; the polynucleotide of interest can be exogenous to the cell it is introduced into; the polynucleotide of interest can be orthologous to the cell it is introduced into; or the polynucleotide of interest can be from a different species than the cell it is introduced into. The term “homologous” in reference to a sequence includes a sequence that is native to the cell. The term “heterologous” in reference to a sequence includes a sequence that originates from a foreign species, or, if from the same species, is substantially modified from its native form in composition and/or locus by deliberate human intervention. The term “exogenous” in reference to a sequence includes a sequence that originates from a foreign species. The term “orthologous” includes a polynucleotide from one species that is functionally equivalent to a known reference sequence in another species (i.e., a species variant). The polynucleotide of interest can be from any organism of interest including, but not limited to, non-human, a rodent, a hamster, a mouse, a rat, a human, a monkey, an agricultural mammal or a non-agricultural mammal. The polynucleotide of interest can further comprise a coding region, a non-coding region, a regulatory region, or a genomic DNA. Thus, the 1st, 2nd, 3rd, 4th, 5th, 6th, 7th, and/or any of the subsequent insert polynucleotides can comprise such sequences.

In one embodiment, the polynucleotide of interest within the insert polynucleotide and/or integrated at the target locus is homologous to a mouse nucleic acid sequence, a human nucleic acid, a non-human nucleic acid, a rodent nucleic acid, a rat nucleic acid, a hamster nucleic acid, a monkey nucleic acid, an agricultural mammal nucleic acid, or a non-agricultural mammal nucleic acid. In still further embodiments, the polynucleotide of interest integrated at the target locus is a fragment of a genomic nucleic acid. In one embodiment, the genomic nucleic acid is a mouse genomic nucleic acid, a human genomic nucleic acid, a non-human nucleic acid, a rodent nucleic acid, a rat nucleic acid, a hamster nucleic acid, a monkey nucleic acid, an agricultural mammal nucleic acid or a non-agricultural mammal nucleic acid or a combination thereof.

In one embodiment, the polynucleotide of interest can range from about 300 nucleotides to about 200 kb as described above. The polynucleotide of interest can be from about 300 nucleotides to about 1 kb, from about 300 nucleotides to about 2 kb, from about 2 kb to about 5 kb, from about 5 kb to about 10 kb, from about 10 kb to about 20 kb, from about 20 kb to about 30 kb, from about 30 kb to about 40 kb, from about 40 kb to about 50 kb, from about 60 kb to about 70 kb, from about 80 kb to about 90 kb, from about 90 kb to about 100 kb, from about 100 kb to about 110 kb, from about 120 kb to about 130 kb, from about 130 kb to about 140 kb, from about 140 kb to about 150 kb, from about 150 kb to about 160 kb, from about 160 kb to about 170 kb, from about 170 kb to about 180 kb, from about 180 kb to about 190 kb, or from about 190 kb to about 200 kb.

The polynucleotide of interest within the insert polynucleotide and/or inserted at the target locus can encode a polypeptide, can encode an miRNA, or it can comprise any regulatory regions or non-coding regions of interest including, for example, a regulatory sequence, a promoter sequence, an enhancer sequence, a transcriptional repressor-binding sequence, or a deletion of a non-protein-coding sequence. In addition, the polynucleotide of interest within the insert polynucleotide and/or inserted at the target locus can encode a protein expressed in the nervous system, the skeletal system, the digestive system, the circulatory system, the muscular system, the respiratory system, the cardiovascular system, the lymphatic system, the endocrine system, the urinary system, the reproductive system, or a combination thereof. In one embodiment, the polynucleotide of interest within the insert polynucleotide and/or inserted at the target locus encodes a protein expressed in a bone marrow or a bone marrow-derived cell. In one embodiment, the polynucleotide of interest within the insert polynucleotide and/or integrated at the target locus encodes a protein expressed in a spleen cell. In still further embodiments, the polynucleotide of interest within the insert polynucleotide and/or inserted at the target locus encodes a protein expressed in a B cell, encodes a protein expressed in an immature B cell or encodes a protein expressed in a mature B cell.

The polynucleotide of interest within the insert polynucleotide and/or integrated at the target locus can encode an extracellular protein or a ligand for a receptor. In specific embodiments, the encoded ligand is a cytokine. Cytokines of interest includes a chemokine selected from CCL, CXCL, CX3CL, and XCL. The cytokine can also comprise a tumor necrosis factor (TNF). In still other embodiments, the cytokine is an interleukin (IL). In one embodiment, the interleukin is selected from IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, and IL-36. In one embodiment, the interleukin is IL-2. In specific embodiments, such polynucleotides of interest within the insert polynucleotide and/or integrated at the target locus are from a human and, in more specific embodiments, can comprise human sequence.

The polynucleotide of interest within the insert polynucleotide and/or integrated at the target locus can encode a cytoplasmic protein or a membrane protein. In one embodiment, the membrane protein is a receptor, such as, a cytokine receptor, an interleukin receptor, an interleukin 2 receptor alpha, an interleukin 2 receptor beta, or an interleukin 2 receptor gamma.

The polynucleotide of interest within the insert polynucleotide and/or integrated at the target locus can comprise a polynucleotide encoding at least a region of a T cell receptor, including the T cell receptor alpha. In specific methods each of the insert polynucleotides comprise a region of the T cell receptor locus (i. e. the T cell receptor alpha locus) such that upon completion of the serial integration, a portion or the entirety of the T cell receptor locus has been integrated at the target locus. Such insert polynucleotides can comprise at least one or more of a variable segment or a joining segment of a T cell receptor locus (i.e. of the T cell receptor alpha locus). In still further the polynucleotide of interest encoding the region of the T cell receptor can be from, for example, a mammal, a non-human mammal, rodent, mouse, rat, a human, a monkey, an agricultural mammal or a domestic mammal polynucleotide encoding a mutant protein.

In other embodiments, the polynucleotide of interest integrated at the target locus encodes a nuclear protein. In one embodiment, the nuclear protein is a nuclear receptor. In specific embodiments, such polynucleotides of interest within the insert polynucleotide and/or integrated at the target locus are from a human and, in more specific embodiments, can comprise human genomic sequence.

The polynucleotide of interest within the insert polynucleotide and/or integrated at the target genomic locus can include a genetic modification in a coding sequence. Such genetic modifications include, but are not limited to, a deletion mutation of a coding sequence or the fusion of two coding sequences.

The polynucleotide of interest within the insert polynucleotide and/or integrated at the target locus can comprise a polynucleotide encoding a mutant protein. In one embodiment, the mutant protein is characterized by an altered binding characteristic, altered localization, altered expression, and/or altered expression pattern. In one embodiment, the polynucleotide of interest within the insert polynucleotide and/or integrated at the target locus includes at least one disease allele, including for example, an allele of a neurological disease, an allele of a cardiovascular disease, an allele of a kidney disease, an allele of a muscle disease, an allele of a blood disease, an allele of a cancer-causing gene, or an allele of an immune system disease. In such instances, the disease allele can be a dominant allele or the disease allele is a recessive allele. Moreover, the disease allele can comprise a single nucleotide polymorphism (SNP) allele. The polynucleotide of interest encoding the mutant protein can be from any organism, including, but not limited to, a mammal, a non-human mammal, rodent, mouse, rat, a human, a monkey, an agricultural mammal or a domestic mammal polynucleotide encoding a mutant protein.

Exemplary disease alleles that can be altered in accordance with the present disclosure include the alleles associated with the following human genetic diseases described in Table 1.

TABLE 1
Genetic Diseases and Defective Genes

| Genetic Disease | Defective Gene |
|---|---|
| AAA syndrome (achalasia- | AAAS |
| addisonianism-alacrima syndrome) | |
| Aarskog-Scott syndrome | FGD1 |
| ABCD syndrome | EDNRB |
| Aceruloplasminemia | CP (3p26.3) |
| Acheiropodia | LMBR1 |
| Achondrogenesis type II | COL2A1 (12q13.11) |
| achondroplasia | FGFR3 (4p16.3) |
| Acute intermittent porphyria | HMBS |
| Adenylosuccinate lyase deficiency | ADSL |
| Adrenoleukodystrophy | ABCD1 (X) |
| ADULT syndrome | TP63 |
| Aicardi-Goutières syndrome | TREX1, RNASEH2A, RNASEH2B, RNASEH2C, |
| | SAMHD1, ADAR, IFIH1 |
| Alagille syndrome | JAG1, NOTCH2 |
| Alexander disease | GFAP |
| alkaptonuria | HGD |
| Alport syndrome | 10q26.13 COL4A3, COL4A4, and COL4A5 |
| Alström syndrome | ALMS1 |
| Alternating hemiplegia of childhood | ATP1A3 |
| Alzheimer's disease | PSEN1, PSEN2, APP, APOEε4 |
| Aminolevulinic acid dehydratase | ALAD |
| deficiency porphyria | |
| Amyotrophic lateral sclerosis - | C9orf72, SOD1, FUS, TARDBP, CHCHD10, |
| Frontotemporal dementia | MAPT |
| Angelman syndrome | UBE3A |
| Apert syndrome | FGFR2 |
| Arthrogryposis-renal dysfunction- | VPS33B |
| cholestasis syndrome | |
| Ataxia telangiectasia | ATM |
| Axenfeld syndrome | PITX2, FOXO1A, FOXC1, PAX6 |
| Beare-Stevenson cutis gyrata | 10q26, FGFR2 |
| syndrome | |
| Beckwith-Wiedemann syndrome | IGF-2, CDKN1C, H19, KCNQ1OT1 |
| biotinidase deficiency | BTD |
| Birt-Hogg-Dubé syndrome | 17 FLCN |
| Björnstad syndrome | BCS1L |
| Brody myopathy | ATP2A1 |
| Brunner syndrome | MAOA |
| CADASIL syndrome | NOTCH3 |
| Canavan disease | ASPA |
| Carpenter Syndrome | RAB23 |
| Cataract | Crygc |
| CDKL5 deficiency disorder | CDKL5 |
| Cerebral dysgenesis-neuropathy- | SNAP29 |
| ichthyosis-keratoderma syndrome | |
| (CEDNIK) | |
| CGD, autosomal recessive | CYBA |
| CGD, X-linked | CYBB |
| Charcot-Marie-Tooth disease | PMP22, MFN2 |
| CHARGE syndrome | CHD7 |
| Chédiak-Higashi syndrome | LYST |
| Cleidocranial dysostosis | RUNX2 |
| Cockayne syndrome | ERCC6, ERCC8 |
| Coffin-Lowry syndrome | X RPS6KA3 |
| Cohen syndrome | COH1 |
| collagenopathy, types II and XI | COL11A1, COL11A2, COL2A1 |
| Congenital insensitivity to pain with | NTRK1 |
| anhidrosis (CIPA) | |
| Cornelia de Lange syndrome (CDLS) | HDAC8, SMC1A, NIPBL, SMA3, RAD21 |
| Cowden syndrome | PTEN |
| CPO deficiency (coproporphyria) | CPOX |
| CRASIL syndrome | HTRA1 |
| Crouzon syndrome | FGFR2, FGFR3 |
| Crouzonodermoskeletal syndrome | FGFR3 |
| (Crouzon syndrome with acanthosis | |
| nigricans) | |
| Cystic fibrosis | CFTR (7q31.2) |
| Cystic fibrosis | CFTR |
| Darier's disease | ATP2A2 |
| Dent's disease (Genetic | Xp11.22 CLCN5, OCRL |
| hypercalciuria) | |
| Denys-Drash syndrome | WT1 |
| Distal hereditary motor neuropathies, | HSPB8, HSPB1, HSPB3, GARS, REEP1, |
| multiple types | IGHMBP2, SLC5A7, DCTN1, TRPV4, SIGMAR1 |
| Distal muscular dystrophy | Dysferlin, TIA1, GNE (gene), MYH7, Titin, |
| | MYOT, MATR3, unknown |
| Dravet syndrome | SCN1A, SCN2A |
| Duchenne muscular dystrophy | Dystrophin |
| Ehlers-Danlos syndrome | COL1A1, COL1A2, COL3A1, COL5A1, COL5A2, |
| | TNXB, ADAMTS2, PLOD1, B4GALT7, DSE |
| Emery-Dreifuss syndrome | EMD, LMNA, SYNE1, SYNE2, FHL1, TMEM43 |
| Epidermolysis bullosa | KRT5, KRT14, DSP, PKP1, JUP, PLEC1, DST, |
| | EXPH5, TGM5, LAMA3, LAMB3, LAMC2, |
| | COL17A1, ITGA6, ITGA4, ITGA3, COL7A1, |
| | FERMT1 |
| Erythropoietic protoporphyria | FECH |
| Fabry disease | GLA (Xq22.1) |
| Familial adenomatous polyposis | APC |
| Familial Creutzfeld-Jakob Disease | PRNP |
| Familial dysautonomia | IKBKAP |
| Fanconi anemia (FA) | FANCA, FANCB, FANCC, FANCD1, FANCD2, |
| | FANCE, FANCF, FANCG, FANCI, FANCJ, |
| | FANCL, FANCM, FANCN, FANCP, FANCS, |
| | RAD51C, XPF |
| Fatal familial insomnia | PRNP |
| Feingold syndrome | MYCN |
| FG syndrome | MED12 |
| Fragile X syndrome | FMR1 |
| Friedreich's ataxia | FXN |
| G6PD deficiency | G6PD |
| Galactosemia | GALT, GALK1, GALE |
| Gaucher disease | GBA (1) |
| Gerstmann-Sträussler-Scheinker | PRNP |
| syndrome | |
| Gillespie syndrome | PAX6 |
| Glutaric aciduria, type I and type 2 | GCDH, ETFA, ETFB, ETFDH |
| GRACILE syndrome | BCS1L |
| Griscelli syndrome | MYO5A, RAB27A, MLPH |
| Hailey-Hailey disease | ATP2C1 (3) |
| Harlequin type ichthyosis | ABCA12 |
| Hemochromatosis, hereditary | HFE, HAMP, HFE2B, TFR2, TF, CP |
| Hemophilia | FVIII |
| Hemophilia A | hF8 |
| Hepatoerythropoietic porphyria | UROD |
| Hereditary Breast Cancer | BRCA1, BRCA2 |
| Hereditary hemorrhagic | ENG, ACVRL1, MADH4 |
| telangiectasia (Osler-Weber-Rendu | |
| syndrome) | |
| Hereditary inclusion body myopathy | GNE, MYHC2A, VCP, HNRPA2B1, HNRNPA1 |
| Hereditary multiple exostoses | EXT1, EXT2, EXT3 |
| Hereditary neuropathy with liability | PMP22 |
| to pressure palsies (HNPP) | |
| Hereditary spastic paraplegia | AP4M1, AP4S1, AP4B1, AP4E1 |
| (infantile-onset ascending hereditary | |
| spastic paralysis) | |
| Hereditary tyrosinemia I | Fah |
| Hermansky-Pudlak syndrome | HPS1, HPS3, HPS4, HPS5, HPS6, HPS7, AP3B1 |
| Heterotaxy | NODAL, NKX2-5, ZIC3, CCDC11, CFC1, SESN1 |
| Homocystinuria | CBS (gene) |
| Hunter syndrome | IDS |
| Huntington disease | HTT |
| Huntington's disease | chromosome 4 HTT gene |
| Hurler syndrome | IDUA |
| Hutchinson-Gilford progeria | LMNA |
| syndrome | |
| Hyperlysinemia | AASS |
| Hyperoxaluria, primary | AGXT, GRHPR, DHDPSL |
| Hypoalphalipoproteinemia (Tangier | ABCA1 |
| disease) | |
| Hypochondrogenesis | COL2A1 |
| Hypochondroplasia | FGFR3 (4p16.3) |
| Incontinentia pigmenti | IKBKG (Xq28) |
| Ischiopatellar dysplasia | TBX4 |
| Jackson-Weiss syndrome | FGFR2 |
| Joubert syndrome | INPP5E, TMEM216, AHI1, NPHP1, CEP290, |
| | TMEM67, RPGRIP1L, ARL13B, CC2D2A, OFD1, |
| | TMEM138, TCTN3, ZNF423, AMRC9 |
| Juvenile primary lateral sclerosis | ALS2 |
| (JPLS) | |
| Kartagener syndrome | DNAI1 |
| Kniest dysplasia | COL2A1 |
| Kosaki overgrowth syndrome | PDGFRB |
| Krabbe disease | GALC |
| Kufor-Rakeb syndrome | ATP13A2 |
| LCAT deficiency | LCAT |
| Lesch-Nyhan syndrome | HPRT (X) |
| Leukemia | CD4* |
| Li-Fraumeni syndrome | TP53 |
| Lynch syndrome | MSH2, MLH1, MSH6, PMS2, PMS1, TGFBR2, |
| | MLH3 |
| Malignant hyperthermia | RYR1 (19q13.2) |
| Maple syrup urine disease | BCKDHA, BCKDHB, DBT, DLD |
| Maroteaux-Lamy syndrome | ARSB |
| McLeod syndrome | XK (X) |
| Mediterranean fever, familial | MEFV |
| MEDNIK syndrome | AP1S1 |
| Menkes disease | ATP7A (Xq21.1) |
| Methylmalonic acidemia | MMAA, MMAB, MMACHC, MMADHC, |
| | LMBRD1, MUT |
| Micro syndrome | RAB3GAP (2q21.3) |
| Microcephaly | ASPM (1q31) |
| MODY | HNF1A, HNF4A, GCK, HNF1B, KCNJ11, ABCC8 |
| Morquio syndrome | GALNS, GLB1 |
| Mowat-Wilson syndrome | ZEB2 (2) |
| Muenke syndrome | FGFR3 |
| Multiple endocrine neoplasia type 1 | MEN1 |
| (Wermer's syndrome) | |
| Multiple endocrine neoplasia type 2 | RET |
| Myostatin-related muscle | MSTN |
| hypertrophy | |
| myotonic dystrophy | DMPK, CNBP |
| Natowicz syndrome | HYAL1 |
| Neonatal diabetes Mellitus NDM | INS1 |
| Neurofibromatosis type II | NF2 (22q12.2) |
| Niemann-Pick disease | SMPD1, NPA, NPB, NPC1, NPC2 |
| Nonketotic hyperglycinemia | GLDC, AMT, GCSH |
| Noonan syndrome | PTPN11, KRAS, SOS1, RAF1, NRAS, HRAS, |
| | BRAF, SHOC2, MAP2K1, MAP2K2, CBL |
| Norman-Roberts syndrome | RELN |
| Omenn syndrome | RAG1, RAG2 |
| Osteogenesis imperfecta | COL1A1, COL1A2, IFITM5 |
| Pantothenate kinase-associated | PANK2 (20p13-p12.3) |
| neurodegeneration | |
| PCC deficiency (propionic acidemia) | PC |
| Pendred syndrome | PDS (7) |
| Peutz-Jeghers syndrome | STK11 |
| Pfeiffer syndrome | FGFR1, FGFR2 |
| Phenylketonuria | PAH |
| Pipecolic acidemia | AASDHPPT |
| Pitt-Hopkins syndrome | TCF4 (18) |
| Polycystic kidney disease | PKD1 (16) or PKD2 (4) |
| Porphyria cutanea tarda (PCT) | UROD |
| Primary ciliary dyskinesia (PCD) | DNAI1, DNAH5, TXNDC3, DNAH11, DNAI2, |
| | KTU, RSPH4A, RSPH9, LRRC50 |
| Protein C deficiency | PROC |
| Protein S deficiency | PROS1 |
| Pseudoxanthoma elasticum | ABCC6 |
| Respiratory distress syndrome of | SFTPC, SFTPB |
| prematurity | |
| Retinitis pigmentosa | RP1, RP2, RPGR, PRPH2, IMPDH1, PRPF31, |
| | CRB1, PRPF8, TULP1, CA4, HPRPF3, ABCA4, |
| | EYS, CERKL, FSCN2, TOPORS, SNRNP200, |
| | PRCD, NR2E3, MERTK, USH2A, PROM1, |
| | KLHL7, CNGB1, TTC8, ARL6, DHDDS, BEST1, |
| | LRAT, SPARA7, CRX |
| Rett syndrome | MECP2 |
| Roberts syndrome | ESCO2 |
| Rubinstein-Taybi syndrome (RSTS) | CREBBP |
| Sandhoff disease | HEXB |
| Sanfilippo syndrome | SGSH, NAGLU, HGSNAT, GNS |
| Schwartz-Jampel syndrome | HSPG2 |
| Shprintzen-Goldberg syndrome | FBN1 |
| Siderius X-linked mental retardation | PHF8 |
| syndrome | |
| Sideroblastic anemia | ABCB7, SLC25A38, GLRX5 |
| Sjogren-Larsson syndrome | ALDH3A2 |
| Sly syndrome | GUSB |
| Smith-Lemli-Opitz syndrome | DHCR7 |
| Spinocerebellar ataxia (types 1-29) | ATXN1, ATXN2, ATXN3, PLEKHG4, SPTBN2, |
| | CACNA1A, ATXN7, ATXN8OS, ATXN10, |
| | TTBK2, PPP2R2B, KCNC3, PRKCG, ITPR1, TBP, |
| | KCND3, FGF14 |
| Spondyloepiphyseal dysplasia | COL2A1 |
| congenita (SED) | |
| SSB syndrome (SADDAN) | FGFR3 |
| Stargardt disease (macular | ABCA4, CNGB3, ELOVL4, PROM1 |
| degeneration) | |
| Stickler syndrome (multiple forms) | COL11A1, COL11A2, COL2A1, COL9A1 |
| Strudwick syndrome | COL2A1 |
| (spondyloepimetaphyseal dysplasia, | |
| Strudwick type) | |
| Tay-Sachs disease | HEXA (15) |
| Tetrahydrobiopterin deficiency | GCH1, PCBD1, PTS, QDPR, MTHFR, DHFR |
| Thanatophoric dysplasia | FGFR3 |
| Treacher Collins syndrome | 5q32-q33.1 (TCOF1, POLR1C, or POLR1D) |
| Tuberous sclerosis complex (TSC) | TSC1, TSC2 |
| Usher syndrome | MYO7A, USH1C, CDH23, PCDH15, USH1G, |
| | USH2A, GPR98, DFNB31, CLRN1 |
| Variegate porphyria | PPOX |
| von Hippel-Lindau disease | VHL |
| von Willebrand disease | VWF |
| Waardenburg syndrome | PAX3, MITF, WS2B, WS2C, SNAI2, EDNRB, |
| | EDN3, SOX10 |
| Weissenbacher-Zweymüller | COL11A2 |
| syndrome | |
| Wilson disease | ATP7B |
| Wollcot-Rallison Syndrome | EIFAK3 |
| Woodhouse-Sakati syndrome | C2ORF37 (2q22.3-q35) |
| X-linked sideroblastic anemia | ALAS2 (X) |
| (XLSA) | |
| Xeroderma pigmentosum | 15 ERCC4 |
| Zellweger syndrome | PEX1, PEX2, PEX3, PEX5, PEX6, PEX10, PEX12, |
| | PEX13, PEX14, PEX16, PEX19, PEX26 |
| α1-antitrypsin deficiency | SERPINEA1 |
| β-thalassemia | HBB |

In one embodiment, the guide RNA binds a 5′ untranslated region of the defective gene or within an intron located 5′ of the defective gene coding sequence.

It is also contemplated herein that genetic animal diseases involving these same disease alleles in animals can also be treated in accordance with the present disclosure.

Moreover, genetic diseases unique to livestock and domestic animals can similarly be treated in accordance with the present disclosure. In one embodiment, the patient is a non-human animal. In another embodiment, the patient is a human.

The polynucleotide of interest within the insert polynucleotide and/or integrated at the target locus can also comprise a regulatory sequence, including for example, an enhancer sequence, or a transcriptional repressor-binding sequence. Such a polynucleotide of interest can be from any organism, including, but not limited to, a mammal, a non-human mammal, rodent, mouse, rat, a human, a monkey, an agricultural mammal or a domestic mammal polynucleotide encoding a mutant protein.

As outlined above, methods and compositions are provided herein to allow for the targeted integration of one or more polynucleotides of interest. Such systems employ a variety of components and for ease of reference, herein the term “targeted integration system” generically refers to all the components required for an integration event (i.e. the various nuclease agents, recognition sites, insert DNA polynucleotides, targeting vectors, target locus, and polynucleotides of interest).

The methods provided herein comprise introducing into a cell one or more polynucleotides or polypeptide constructs including the various components of the targeted integration system. The term “introducing” includes presenting to the cell the sequence (polypeptide or polynucleotide) in such a manner that the sequence gains access to the interior of the cell. The methods provided herein do not depend on a particular method for introducing any component of the targeted integration system into the cell, only that the polynucleotide gains access to the interior of a least one cell. Methods for introducing polynucleotides into various cell types are known in the art and include, but are not limited to, stable transfection methods, transient transfection methods, and virus-mediated methods.

In some embodiments, the cells employed in the methods and compositions have a DNA construct stably incorporated into their genome. “Stably incorporated” or “stably introduced” means the introduction of a polynucleotide into the cell such that the nucleotide sequence integrates into the genome of the cell and is capable of being inherited by progeny thereof. Any protocol may be used for the stable incorporation of the DNA constructs or the various components of the targeted integration system.

Transfection protocols as well as protocols for introducing polypeptides or polynucleotide sequences into cells may vary. Non-limiting transfection methods include chemical-based transfection methods include the use of liposomes; nanoparticles; calcium phosphate (Graham et al., Virology, 52(2):456-67 (1973), Bacchetti et al., Proc Natl Acad Sci USA, 74(4):1590-4 (1977), and Kriegler, M, Transfer and Expression: A Laboratory Manual. New York: W. H. Freeman and Company. pp. 96-97 (1991), all of which are hereby incorporated by reference in their entirety; dendrimers; or cationic polymers such as DEAE-dextran or polyethylenimine. Non-chemical methods include electroporation; Sono-poration; and optical transfection. Particle-based transfection include the use of a gene gun, magnet assisted transfection (Bertram, J., Current Pharmaceutical Biotechnology, 7:277-28 (2006), which is hereby incorporated by reference in its entirety). Viral methods can also be used for transfection. Any suitable viral vector can be utilized including, without limitation, adeno-associated virus, adenovirus, and lentivirus vectors.

In one embodiment, the nuclease agent is introduced into the cell simultaneously with the targeting vector or the large targeting vector (LTVEC). Alternatively, the nuclease agent is introduced separately from the targeting vector or the LTVEC over a period of time. In one embodiment, the nuclease agent is introduced prior to the introduction of the targeting vector or the LTVEC, while in other embodiments, the nuclease agent is introduced following introduction of the targeting vector or the LTVEC.

Non-human mammalian animals can be generated employing the various methods disclosed herein. Such methods include (1) integrating one or more polynucleotide of interest at the target locus of a pluripotent cell of the non-human animal to generate a genetically modified pluripotent cell including the insert polynucleotide in the targeted locus employing the methods disclosed herein; (2) selecting the genetically modified pluripotent cell having the one or more polynucleotides of interest at the target locus; (3) introducing the genetically modified pluripotent cell into a host embryo of the non-human animal at a pre-morula stage; and (4) implanting the host embryo including the genetically modified pluripotent cell into a surrogate mother to generate an F0 generation derived from the genetically modified pluripotent cell. The non-human animal can be a non-human mammal, a rodent (e.g., a mouse, a rat, a hamster), a monkey, an agricultural mammal or a domestic mammal. The pluripotent cell can be a human ES cell, a human iPS cell, a non-human ES cell, a rodent ES cell (e.g., a mouse ES cell, a rat ES cell, or a hamster ES cell), a monkey ES cell, an agricultural mammal ES cell or a domesticated mammal ES cell. See, e.g., U.S. Publication No. 2014/0235933; U.S. Publication No. 2014/0310828; and Tong et al., Nature, 467(7312):211-213 (2010), each of which is herein incorporated by reference in its entirety.

Nuclear transfer techniques can also be used to generate the non-human mammalian animals. Briefly, methods for nuclear transfer include the steps of: (1) enucleating an oocyte; (2) isolating a donor cell or nucleus to be combined with the enucleated oocyte; (3) inserting the cell or nucleus into the enucleated oocyte to form a reconstituted cell; (4) implanting the reconstituted cell into the womb of an animal to form an embryo; and (5) allowing the embryo to develop. In such methods oocytes are generally retrieved from deceased animals, although they may be isolated also from either oviducts and/or ovaries of live animals. Oocytes can be matured in a variety of medium known to those of ordinary skill in the art prior to enucleation. Enucleation of the oocyte can be performed in a number of manners well known to those of ordinary skill in the art. Insertion of the donor cell or nucleus into the enucleated oocyte to form a reconstituted cell is usually by microinjection of a donor cell under the zona pellucida prior to fusion. Fusion may be induced by application of a DC electrical pulse across the contact/fusion plane (electrofusion), by exposure of the cells to fusion-promoting chemicals, such as polyethylene glycol, or by way of an inactivated virus, such as the Sendai virus. A reconstituted cell is typically activated by electrical and/or non-electrical means before, during, and/or after fusion of the nuclear donor and recipient oocyte. Activation methods include electric pulses, chemically induced shock, penetration by sperm, increasing levels of divalent cations in the oocyte, and reducing phosphorylation of cellular proteins (as by way of kinase inhibitors) in the oocyte. The activated reconstituted cells, or embryos, are typically cultured in medium well known to those of ordinary skill in the art and then transferred to the womb of an animal. See, for example, US20080092249, WO/1999/005266A2, US20040177390, WO/2008/017234A1, and U.S. Pat. No. 7,612,250, each of which is herein incorporated by reference. In one embodiment, the introducing is carried out by microinjection, electroporation, or hydrodynamic injection.

In some embodiments, targeted mammalian ES cells (i.e., from humans as well as non-human mammals, rodents (e.g., mice, rats, or hamsters), agricultural mammals, domestic mammals, monkeys, etc.) including various genetic modifications as described herein are introduced into a pre-morula stage embryo from a corresponding organism, e.g., an 8-cell stage mouse embryo, via the VELOCIMOUSE™ method (see, e.g., U.S. Pat. Nos. 7,576,259, 7,659,442, 7,294,754, and U.S. 2008-0078000 A1, all of which are incorporated by reference herein in their entireties). The non-human mammalian embryo including the genetically modified ES cells is incubated until the blastocyst stage and then implanted into a surrogate mother to produce an F0. In some other embodiments, targeted mammalian ES cells including various genetic modifications as described herein are introduced into a blastocyst stage embryo. Non-human mammals bearing the genetically modified locus can be identified via modification of allele (MOA) assay as described herein. The resulting F0 generation non-human mammal derived from the genetically modified ES cells is crossed to a wild-type non-human mammal to obtain F1 generation offspring. Following genotyping with specific primers and/or probes, F1 non-human mammals that are heterozygous for the genetically modified locus are crossed to each other to produce non-human mammals that are homozygous for the genetically modified locus.

The various methods described herein employ a locus targeting system in a cell. Such cells include eukaryotic cells such as mammalian cells, including, but not limited to a mouse cell, a rat cell, a rabbit cell, a pig cell, a bovine cell, a deer cell, a sheep cell, a goat cell, a cat cell, a dog cell, a ferret cell, a primate (e.g., human, marmoset, rhesus monkey) cell, and the like and cells from domesticated mammals or cells from agricultural mammals. Some cells are human. Some cells are non-human, particularly non-human mammalian cells. In some embodiments, for those mammals for which suitable genetically modifiable pluripotent cells are not readily available, other methods are employed to reprogram somatic cells into pluripotent cells, e.g., via introduction into somatic cells of a combination of pluripotency-inducing factors, including, but not limited to, Oct3/4, Sox2, KLF4, Myc, Nanog, LIN28, and Glis1.

In one embodiment, the eukaryotic cell is a pluripotent cell. In one embodiment, the pluripotent cell is an embryonic stem (ES) cell. The term “embryonic stem cell” or “ES cell” includes an embryo-derived totipotent or pluripotent cell that is capable of undifferentiated proliferation in vitro, and is capable of contributing to any tissue of the developing embryo upon introduction into an embryo. The term “pluripotent cell” includes an undifferentiated cell that possesses the ability to develop into more than one differentiated cell type. The term “germline” in reference to a polynucleotide sequence includes a nucleic acid sequence that can be passed to progeny.

The pluripotent cell can be a human or non-human ES cell, or an induced pluripotent stem (iPS) cell. In one embodiment, the induced pluripotent (iPS) cell is derived from a fibroblast. In specific embodiments, the induced pluripotent (iPS) cell is derived from a human fibroblast. In some embodiments, the pluripotent cell is a hematopoietic stem cell (HSC), a neuronal stem cell (NSC), or an epiblast stem cell. The pluripotent cell can also be a developmentally restricted progenitor cell.

In other embodiments, the mammalian cell can immortalized mouse cell, rat cell or human cell. In one embodiment, the mammalian cell is a human fibroblast, while in other embodiments, the mammalian cell is a cancer cell, including a human cancer cell.

In still further embodiments, the mammal is a human and the targeting is carried out using an ex vivo human cell. In one embodiment, the cell is present in an individual or the patient. In one embodiment, the cell is ex vivo. In one embodiment, the cell is a mitotic or post-mitotic cell. In one embodiment, the cell is a pluripotent stem cell, a somatic stem cell, a de-differentiated cell, or a zygote. In one embodiment, the cell is a zygote obtained via in vitro fertilization. In one embodiment, the selecting step described herein further includes selecting cells that also lack insertions or deletions at the replacement coding sequence integration site. In one embodiment, the methods described herein further include isolating the selected cells and culturing the isolated cells to prior to introducing. In another embodiment, the coding sequence of the DNA template is intronless. Alternatively, the coding sequence of the DNA template may, in one embodiment, include one or more introns.

In one embodiment, the mammalian cell is a human cell isolated from a patient having a disease and/or includes a human polynucleotide encoding a mutant protein. In one embodiment, the mutant human protein is characterized by an altered binding characteristic, altered localization, altered expression, and/or altered expression pattern. In one embodiment, the human nucleic acid sequence includes at least one human disease allele. In one embodiment, the human nucleic acid sequence includes at least one human disease allele. In one embodiment, the human disease allele is an allele of a neurological disease. In one embodiment, the human disease allele is an allele of a cardiovascular disease. In one embodiment, the human disease allele is an allele of a kidney disease. In one embodiment, the human disease allele is an allele of a muscle disease. In one embodiment, the human disease allele is an allele of a blood disease. In one embodiment, the human disease allele is an allele of a cancer-causing gene. In one embodiment, the human disease allele is an allele of an immune system disease. In one embodiment, the human disease allele is a dominant allele. In one embodiment, the human disease allele is a recessive allele. In one embodiment, the human disease allele includes a single nucleotide polymorphism (SNP) allele.

In one embodiment, the methods described herein further include obtaining the cell from an individual prior to said providing or from the patient prior to said repairing.

In one embodiment, the methods described herein further include selecting cells having corrected the gene defect; and introducing selected cells into the individual or the patient. In one embodiment, the one or more vectors or the one or more non-viral delivery vehicles are administered to a patient.

Provided herein are polynucleotides or nucleic acid molecules including the various components of the targeted integration system provided herein (i.e. nuclease agents, recognition sites, insert polynucleotides, polynucleotides of interest, targeting vectors, selection markers and other components).

The terms “polynucleotide,” “polynucleotide sequence,” “nucleic acid sequence,” and “nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Polynucleotides can comprise deoxyribonucleotides and ribonucleotides include both naturally occurring molecules and synthetic analogues, and any combination these. The polynucleotides provided herein also encompass all forms of sequences including, but not limited to, single-stranded forms, double-stranded forms, hairpins, stem-and-loop structures, and the like.

Further provided are recombinant polynucleotides including the various components of the targeted integration system. The terms “recombinant polynucleotide” and “recombinant DNA construct” are used interchangeably herein. A recombinant construct includes an artificial or heterologous combination of nucleic acid sequences, e.g., regulatory and coding sequences that are not found together in nature. In other embodiments, a recombinant construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that is used to transform the host cells as is well known to those skilled in the art. For example, a plasmid vector can be used. Genetic elements required to successfully transform, select, and propagate host cells and including any of the isolated nucleic acid fragments are provided herein. Screening may be accomplished by Southern analysis of DNA, Northern analysis of mRNA expression, immunoblotting analysis of protein expression, or phenotypic analysis, among others.

In specific embodiments, one or more of the components of the targeted integration system described herein can be provided in an expression cassette for expression in a prokaryotic cell, a eukaryotic cell, a bacterial, a yeast cell, or a mammalian cell or other organism or cell type of interest. The cassette can include 5′ and 3′ regulatory sequences operably linked to a polynucleotide provided herein. “Operably linked” includes a functional linkage between two or more elements. For example, an operable linkage between a polynucleotide of interest and a regulatory sequence (i.e., a promoter) is a functional link that allows for expression of the polynucleotide of interest. Operably linked elements may be contiguous or non-contiguous. When used to refer to the joining of two protein coding regions, operably linked means that the coding regions are in the same reading frame. In another instance, a nucleic acid sequence encoding a protein may be operably linked to regulatory sequences (e.g., promoter, enhancer, silencer sequence, etc.) so as to retain proper transcriptional regulation. In one instance, a nucleic acid sequence of an immunoglobulin variable region (or V(D)J segments) may be operably linked to a nucleic acid sequence of an immunoglobulin constant region so as to allow proper recombination between the sequences into an immunoglobulin heavy or light chain sequence.

The expression cassette may additionally contain at least one additional polynucleotide of interest to be co-introduced into the organism. Alternatively, the additional polynucleotide of interest can be provided on multiple expression cassettes. Such an expression cassette is provided with a plurality of restriction sites and/or recombination sites for insertion of a recombinant polynucleotide to be under the transcriptional regulation of the regulatory regions. The expression cassette may additionally contain selection marker genes.

The expression cassette can include in the 5′-3′ direction of transcription, a transcriptional and translational initiation region (i.e., a promoter), a recombinant polynucleotide provided herein, and a transcriptional and translational termination region (i.e., termination region) functional in mammalian cell or a host cell of interest. The regulatory regions (i.e., promoters, transcriptional regulatory regions, and translational termination regions) and/or a polynucleotide provided herein may be native/analogous to the host cell or to each other. Alternatively, the regulatory regions and/or a polynucleotide provided herein may be heterologous to the host cell or to each other. For example, a promoter operably linked to a heterologous polynucleotide is from a species different from the species from which the polynucleotide was derived, or, if from the same/analogous species, one or both are substantially modified from their original form and/or locus, or the promoter is not the native promoter for the operably linked polynucleotide. Alternatively, the regulatory regions and/or a recombinant polynucleotide provided herein may be entirely synthetic.

The termination region may be native with the transcriptional initiation region, may be native with the operably linked recombinant polynucleotide, may be native with the host cell, or may be derived from another source (i.e., foreign or heterologous) to the promoter, the recombinant polynucleotide, the host cell, or any combination thereof.

In preparing the expression cassette, the various DNA fragments may be manipulated, so as to provide for the DNA sequences in the proper orientation. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the expression cassettes provided herein. The promoters can be selected based on the desired outcome. It is recognized that different applications can be enhanced by the use of different promoters in the expression cassettes to modulate the timing, location and/or level of expression of the polynucleotide of interest. Such expression constructs may also contain, if desired, a promoter regulatory region (e.g., one conferring inducible, constitutive, environmentally- or developmentally-regulated, or cell- or tissue-specific/selective expression), a transcription initiation start site, a ribosome binding site, an RNA processing signal, a transcription termination site, and/or a polyadenylation signal.

The expression cassette containing the polynucleotides provided herein can also comprise a selection marker gene for the selection of transformed cells. Selection marker genes are utilized for the selection of transformed cells or tissues.

Where appropriate, the sequences employed in the methods and compositions (i.e., the polynucleotide of interest, the nuclease agent, etc.) may be optimized for increased expression in the cell. That is, the genes can be synthesized using codons preferred in a given cell of interest including, for example, mammalian-preferred codons, human-preferred codons, rodent-preferred codon, mouse-preferred codons, rat-preferred codons, etc. for improved expression.

The methods and compositions provided herein employ a variety of different components of the targeted integration system (i.e. nuclease agents, recognition sites, insert polynucleotides, polynucleotides of interest, targeting vectors, selection markers and other components). It is recognized throughout the description that some components of the targeted integration system can have active variants and fragments. Such components include, for example, nuclease agents (i.e. engineered nuclease agents), nuclease agent recognition sites, polynucleotides of interest, target sites and corresponding homology arms of the targeting vector. Biological activity for each of these components is described elsewhere herein. In one embodiment, the providing or repairing described herein is carried out by introducing into the cell one or more vectors including the first nucleic acid molecule, the second nucleic acid molecule, and the DNA template.

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” means any sequence comparison program that, for any two sequences in question, generates an alignment having identical nucleotide or amino acid residue matches and an identical percent sequence identity when compared to the corresponding alignment generated by GAP Version 10. In one embodiment, the DNA template further includes an identical or nearly identical nucleotide sequence as the target binding site.

A third aspect relates to a system for correcting a gene defect in a cell. The system includes:

a first vector that includes a first nucleic acid molecule encoding a Cas protein;

a second vector that includes a DNA template including a replacement coding sequence, which encodes a non-defective protein, and a transcription terminator sequence,

wherein one of the first and second vectors includes a nucleic acid molecule encoding a guide RNA that is capable of base-pairing with a region of a defective gene between a promoter and a coding sequence thereof.

In one embodiment, the first and second vectors comprise viral vectors in accordance with the viral vectors described herein. In one embodiment, the first and second vectors are selected from the group consisting of adeno-associated virus, adenovirus, and lentivirus vectors.

A fourth aspect relates to system for correcting a gene defect in a cell. The system includes: one or more non-viral delivery vehicles that comprise a Cas protein, or a nucleic acid molecule encoding the Cas protein, a guide RNA that is capable of base-pairing with a region of a defective gene between a promoter and a coding sequence thereof, and a DNA template including a replacement coding sequence, which encodes a non-defective protein, and a transcription terminator sequence.

This aspect is carried out in accordance with the previously described aspects.

In one embodiment, the one or more non-viral delivery vehicles include the Cas protein, the guide RNA, and the DNA template. In another embodiment, the one or more non-viral delivery vehicles include mRNA encoding the Cas protein, the guide RNA, and the DNA template. In one embodiment, the one or more non-viral delivery vehicles include lipid-like nanoparticles, inorganic nanoparticles, or cell-penetrating peptides. In another embodiment, the coding sequence of the DNA template is intronless. In another embodiment, the coding sequence of the DNA template includes one or more introns. The defective gene may, in one embodiment, be selected from any defective genes described above with reference to Table 1.

In one embodiment, the guide RNA binds is a 5′ untranslated region of the defective gene or within an intron located 5′ of the defective gene coding sequence. In one embodiment, the Cas protein is a Cas9 protein. In one embodiment, the Cas9 protein is selected from Streptococcus pyogenes Cas9 and Streptococcus aureus Cas9. In one embodiment, the guide RNA includes one or more modified bases or a modified backbone. In one embodiment, the non-defective protein is a wild-type variant or a modified variant having improved activity relative to wild-type. In one embodiment, the DNA template further includes an identical or nearly identical nucleotide sequence as the target binding site.

A further aspect relates to a composition that includes a system in accordance with the systems described herein.

A further aspect relates to an ex vivo modified cell prepared according to the methods described herein.

A further aspect relates to an ex vivo modified cell having a repair of a gene defect, the modified cell including a promoter and a coding sequence for a defective gene product, and a replacement coding sequence and transcription terminator inserted into a region between the promoter and the coding sequence for the defective gene product via NHEJ repair pathway, whereby the modified cell expresses a non-defective protein encoded by the replacement coding sequence under control of the promoter but not the defective gene product.

This aspect is carried out in accordance with the previously described aspects.

In one embodiment, the ex vivo modified cell is a mitotic or post-mitotic cell. In one embodiment, the ex vivo modified cell is a pluripotent stem cell, a somatic stem cell, a de-differentiated cell, or a zygote. In one embodiment, the ex vivo modified cell is a zygote obtained via in vitro fertilization. In one embodiment, the ex vivo modified cell lacks insertions or deletions at the replacement coding sequence integration site. In one embodiment, the coding sequence of the DNA template of the ex vivo modified cell is intronless. In one embodiment, the coding sequence of the DNA template of the ex vivo modified cell includes one or more introns. In one embodiment, the defective gene is one of those listed in Table 1 described herein. In another embodiment, the non-defective protein is a wild-type variant or a modified variant having improved activity relative to wild-type.

A further aspect relates to a composition including an aqueous delivery vehicle and the ex vivo modified cell according to any of those described herein.

In one embodiment, the composition includes at least 1000 ex vivo modified cells.

A further aspect relates to a method of preparing a chimeric antigen receptor T cell. The method includes:

providing in an isolated T cell (i) a Cas protein or a first nucleic acid molecule encoding the Cas protein, (ii) a guide RNA that is capable of base-pairing with a region of a native gene between a promoter and a coding sequence thereof, or a second nucleic acid encoding the guide RNA, and (iii) a DNA template including a replacement coding sequence, which encodes a heterologous antigen receptor, and a transcription terminator sequence,

wherein upon binding of the guide RNA to a 5′ untranslated region of the native gene and cleavage of the 5′ untranslated region by the Cas protein, the DNA template is inserted into the genome of the cell via NHEJ repair pathway to allow for expression of the heterologous antigen receptor under control of the native gene promoter while simultaneously blocking the expression of the native gene product.

This aspect is carried out in accordance with the previously described aspects.

In one embodiment, the method further includes obtaining the T cell from an individual prior to said providing. In one embodiment, the method further includes selecting T cells that express the heterologous antigen receptor but not the native gene product. In another embodiment, the method includes introducing selected cells into the individual.

A further aspect relates to an ex vivo modified T cell prepared according to any method described herein.

A further aspect relates to an ex vivo modified T-cell that expresses a chimeric antigen receptor, the modified T cell including a promoter and a coding sequence for native gene product, and a replacement coding sequence and transcription terminator inserted into a region between the promoter and the coding sequence for the native gene product via NHEJ repair pathway, whereby the modified T cell expresses a chimeric antigen receptor encoded by the replacement coding sequence under control of the promoter but not the native gene product.

In one embodiment, the T-cell lacks insertions or deletions at the replacement coding sequence integration site. In another embodiment, the replacement coding sequence is intronless. In yet another embodiment, the replacement coding sequence includes one or more introns. In another embodiment, the native gene is selected from the group of PD-1, CD95/Fas, or an HLA (class I) receptor.

A further aspect relates to a composition including an aqueous delivery vehicle and the ex vivo modified T-cell as described herein.

In one embodiment, the composition includes at least 1000 ex vivo modified T-cells.

### EXAMPLES

The following Examples are presented to illustrate various aspects of the disclosure, but are not intended to limit the scope of the claimed invention.

### Materials and Methods for Examples

Trans genic mice—Perk KO (c.1584C>A; p. Cys528X)—A transgenic mouse model with a nonsense mutation in the exon 9 of mouse Perk gene (c.1584C>A; p.Cys528X) was generated by CRISPR/Cas9-mediated genome editing via HDR in mouse zygote with a 200 nt single-stranded oligodeoxynucleotides (ssODN) template containing one nonsense mutation and four synonymous mutations. SpCas9 mRNA (5meC, Ψ) was purchased from TriLink (San Diego, Calif.). In vitro transcription and purification of mPERKex9-sgRNA were as previously described (Yang et al., Nat. Protoc., 9:1956-1968 (2014), which is hereby incorporated by reference in its entirety). Repair template (200 nt ssODN, 4 nmole Ultramer DNA Oligo) was purchase from Integrated DNA Technologies (IDT, Coralville, Iowa)

(SEQ ID NO: 3)
cagcccccactacagcaagaacatccgcaagaaggaccctatcctcctg
ctgcactggtggaaggagatattcgggacgatcctgctttgAatcgtGg
ccacAacGttTatcgtgcgcaggcttttccatcctcagccccacagggt
aagatgctctgtcaacctaatgtgcttccaagtggttgctgtgtaggaa
acct.

A nonsense mutation was introduced by a C to A mutation on the ssODN template, 14 bp from the Cas9/sgRNA cleavage. Three synonymous mutations were designed 2 bp, 5 bp and 8 bp from PAM site to prevent re-excision of the HDR repaired genome. SpCas9 mRNA, sgRNA, and ssODN were sent to the Harvard Genome Modification Facility for microinjection into C57BL/6J zygotes and implantation into pseudo pregnant females. Fifty-seven individuals survived to weaning age from one injection experiment; thirteen individuals carried the Perk KO allele (C528X).

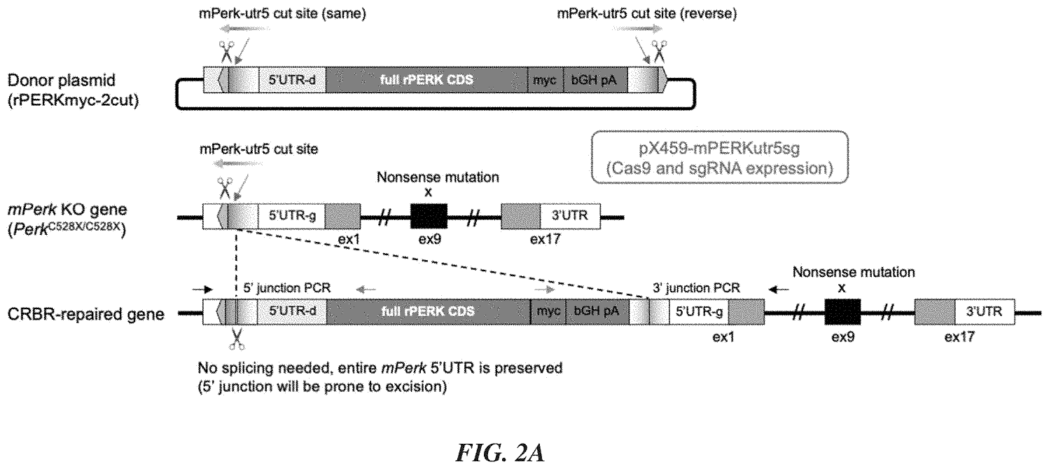

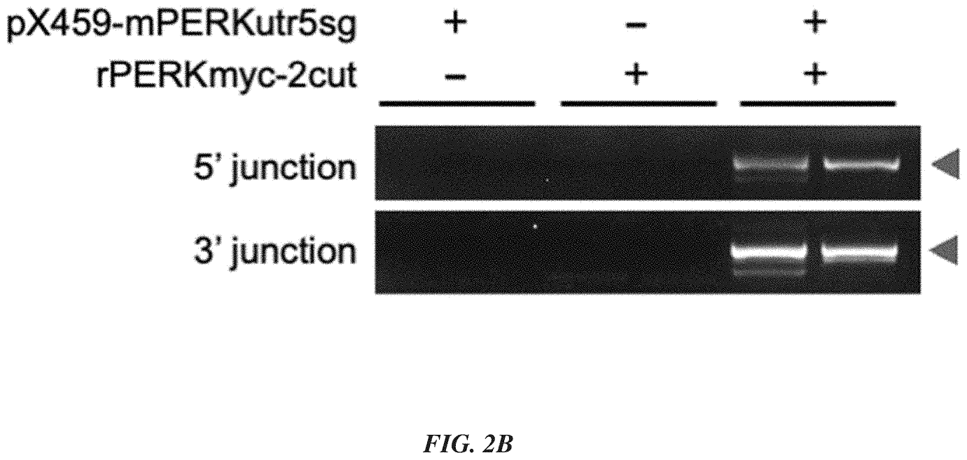

Transgenic mice—rPerk-CRBR (rPERKmyc integration at 5′UTR of mPerk)—A transgenic mouse model with rPERK-CRBR allele (rPERKmyc integration at 5′UTR of mPerk) was generated by CRBR-mediated gene editing in mouse zygote. A rPERK CDS with a myc tag at the C-terminus was designed to integrate into mouse Perk 5′UTR region using CRBR strategy as described in Results (FIG. 2A). SpCas9 protein was purchased from IDT. A synthetic mPERKutr5-sgRNA (see Construction of plasmids for sgRNA sequence) was purchased from Synthego (Redwood City, Calif.). The rPERKmyc-2cut donor plasmid was constructed as described in Construction of plasmids. The SpCas9 protein, sgRNA, and the rPERKmyc-2cut donor plasmid were sent to Harvard Genome Modification Facility for microinjection into C57BL/6J zygotes and implantation into pseudo pregnant females. Twenty-one individuals survived to weaning age from two injection experiments; one individual carried the CRBR-edited allele (rPERK-CRBR).

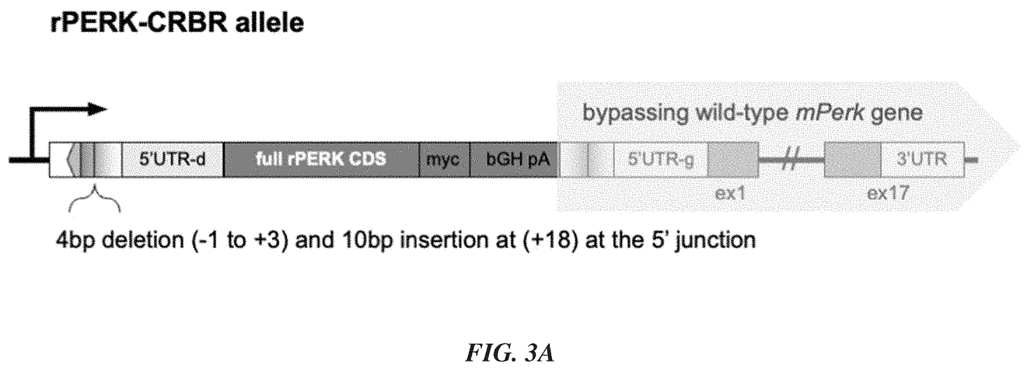

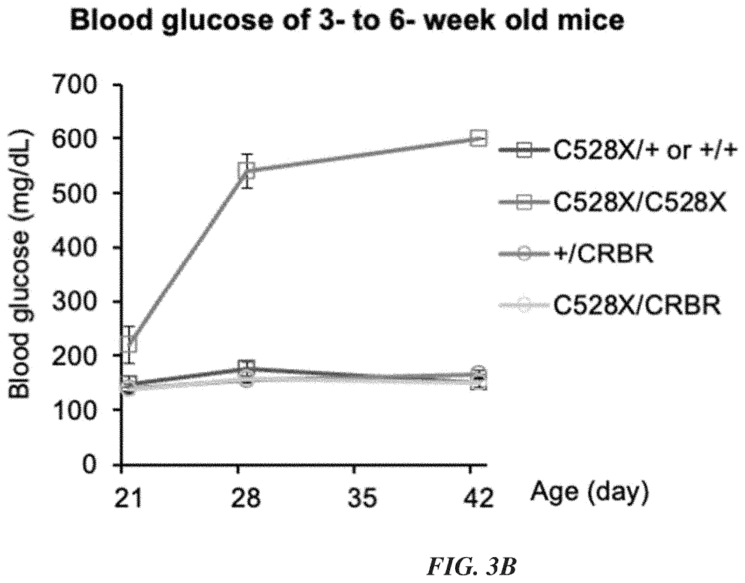

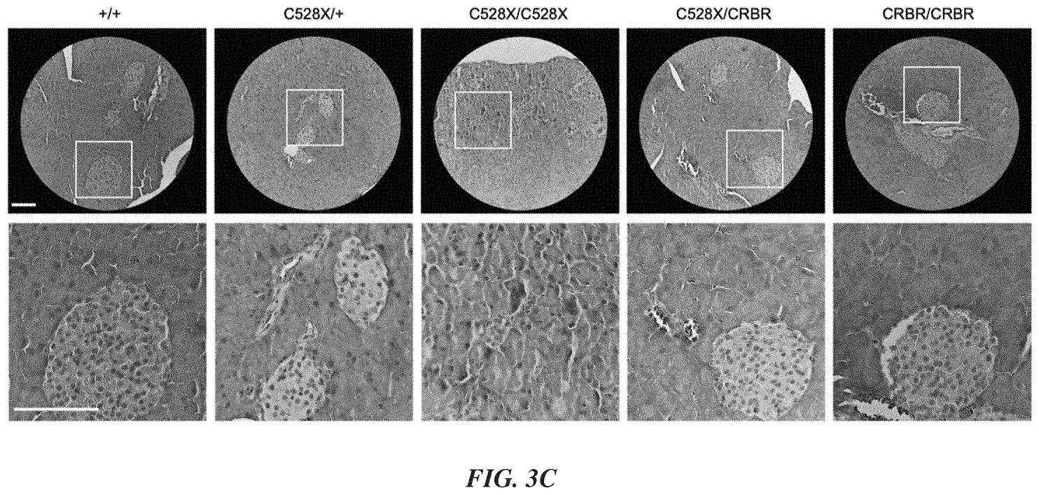

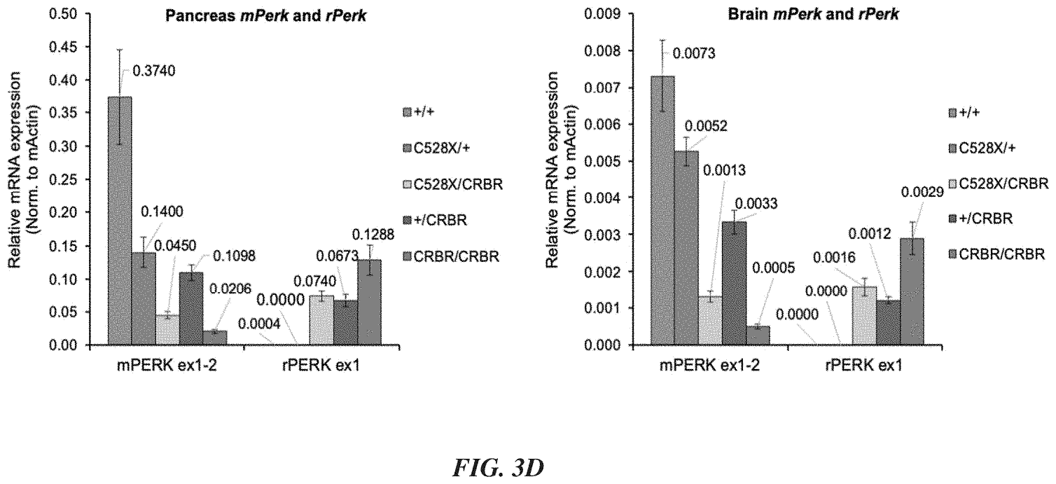

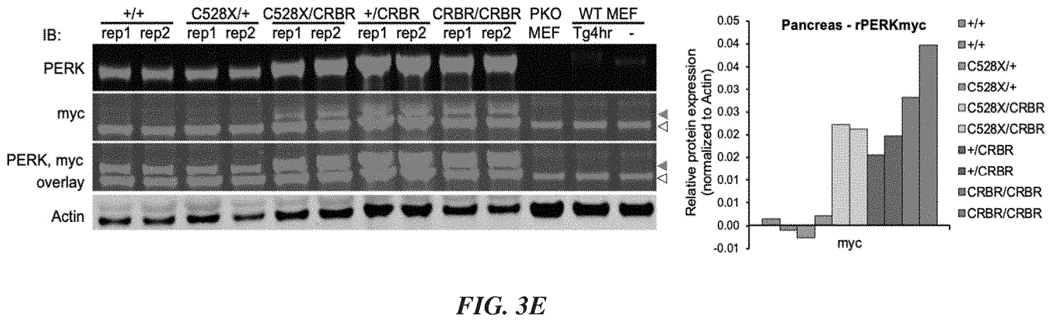

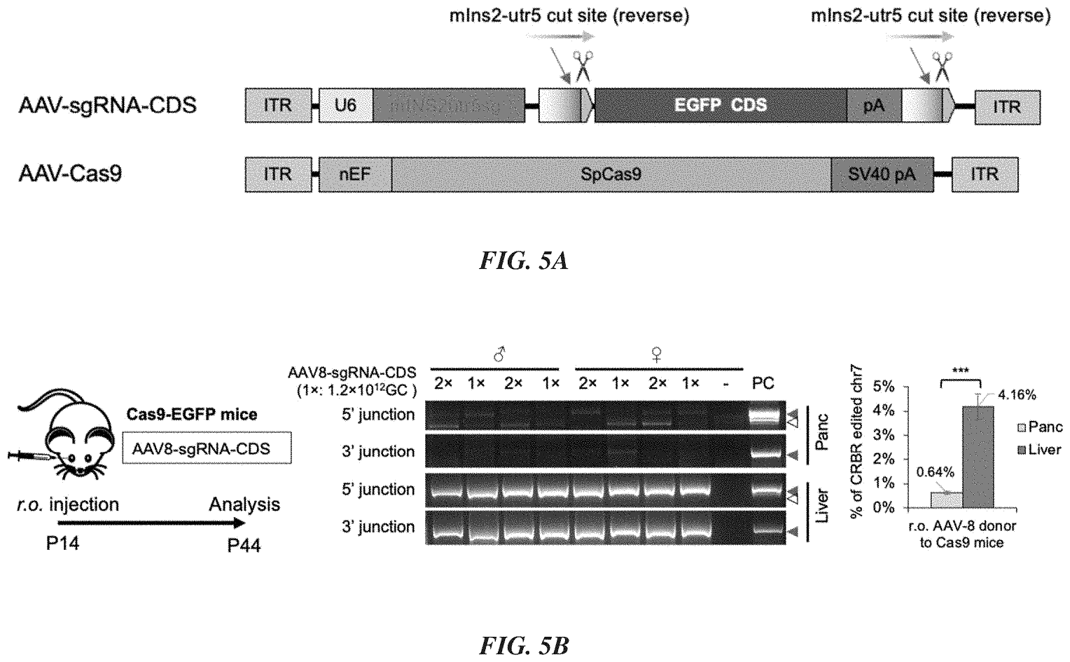

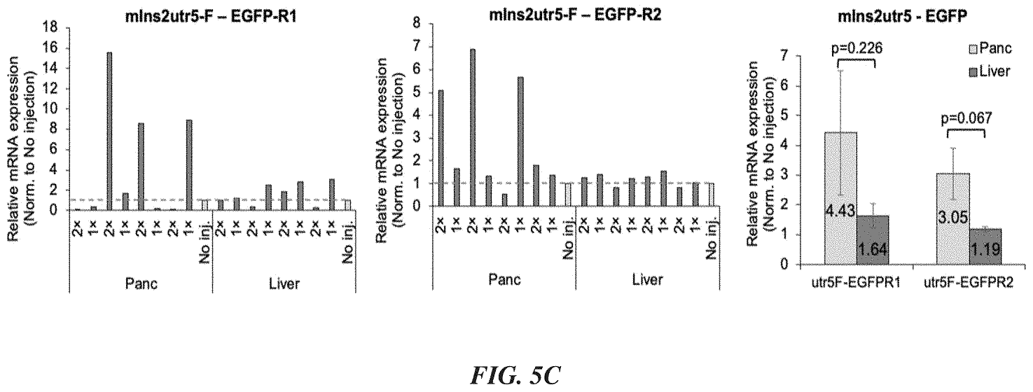

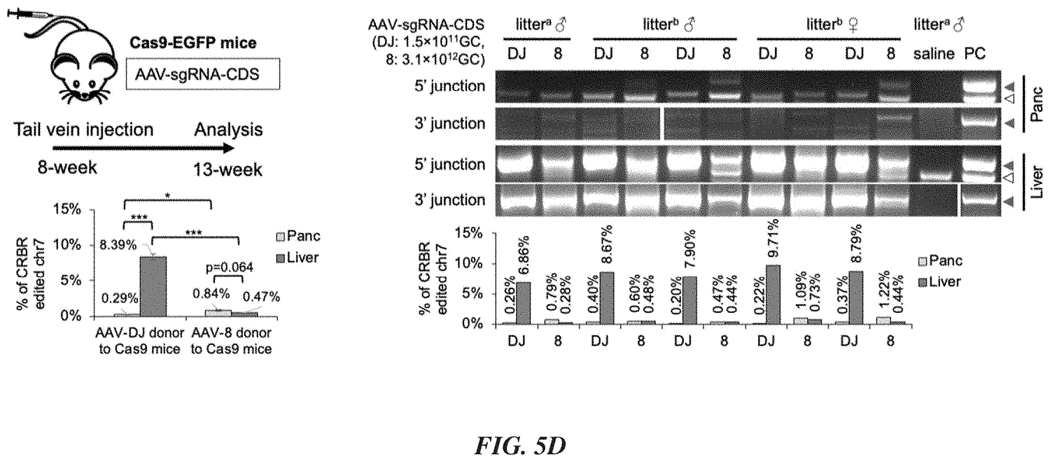

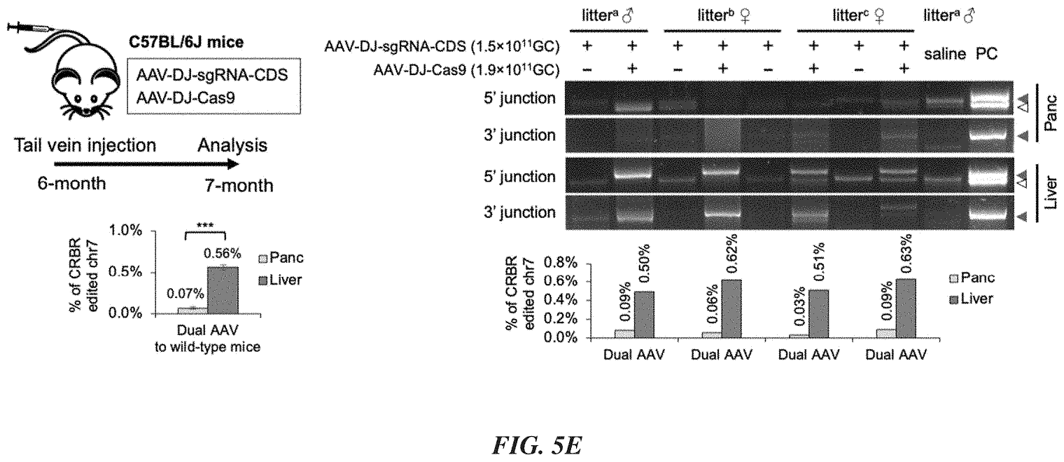

Genetic strains—B6J.129(Cg)-Gt(ROSA)^{26Sortm1.1(CAG-cas9*, -EGFP)Fezh/}J (Cas9-EGFP), C57BL/6J (wild-type) and 129S1/SvImJ (wild-type) mice were purchased from the Jackson Laboratory. The generation of the Perk KO allele (Δex7-9) had been previously described (Zhang et al., Molecular and Cellular Biology, 22:3864-3874 (2002), which is hereby incorporated by reference in its entirety). Perk^{Δex7-9/+} strain (used to cross with Perk^{rPERK-CRBR/+}) was congenic for C57BL/6J. Perk^{C528X/+}, Perk^{rPERK-CRBR/+} and offspring (FIGS. 3A-3E) were of mixed C57BL/6J and 129S1/SvImJ background. The Cas9-EGFP strain (FIGS. 5A-5E) was of mixed C57BL/6J and 129S1/SvImJ background. Blood glucose was measured from tail blood using OneTouch UltraMIni glucometer (LifeScan, Malvern, Pa.). Mice were sacrificed by CO₂ asphyxiation. All animal studies were reviewed and approved by the Institutional Animal Care and Use Committee (IACUC) of the Pennsylvania State University.

Construction of plasmids—The vectors expressing SpCas9 and sgRNAs targeting mPERK, mIns2 and hINS genes were cloned into the pX459 plasmid (pSpCas9(BB)-2A-Puro V2.0, (Addgene, Watertown, Mass., plasmid #62988, deposited by Feng Zhang) as previously described (Ran et al., Nature Protocols, 8:2281-2308 (2013), which is hereby incorporated by reference in its entirety). The Cas9/sgRNA genomic target sequences (20 nt+PAM (bold)) on sense (+) or antisense strand (−) used in this study include:

(SEQ ID NO: 4)
mPerk-ex9, CCTGCGCACGATGAAGGTCGTGG (−);
(SEQ ID NO: 5)
mPerk-in6, TAGTTCGGGATCGCCACATGAGG (−);
(SEQ ID NO: 6)
mPerk-utr5, AGACATCGCCCATTGAGCGAGGG (−);
(SEQ ID NO: 7)
mIns2-utr5, TGTAGCGGATCACTTAGGGCTGG (−);
(SEQ ID NO: 8)
hINS-in1 (or hINS-in1-Reverse),
GCCCCAGCTCTGCAGCAGGGAGG (+);
(SEQ ID NO: 9)
hINS-in1-Same, TGGGCTCGTGAAGCATGTGGGGG (+).

Each of these target sequences were determined by Surveyor Assay (IDT) or T7 Endonuclease I (T7E1) Assay (New England Biolabs, Ipswich, Mass.) from 2-3 candidates with top on-target scores identified from crispr.mit.edu or benchling.com/crispr/. To construct rPERKex7-17-2cut, rPERKex7-17CDS-bGHpA was amplified by mega primer adding 3′ cut site to the amplicon from pcDNA-rPERK (in house) and TA-cloned into pCR2.1 (Invitrogen, Carlsbad, Calif.), followed by subcloning of the 3′ part of mPerk intron 6 and a 5′ cut site by PCR amplification into the pCR2.1-rPERKex7-17CDS-bGHpA-3pCUT. A rPERK-2cut was first generated by cloning ITR-mPERKutr5-rPERK-CDS-bGHpA-3×3pCUT-ITR into pBluescript II KS (+) through PciI and SalI (synthesized by GenScript, Piscataway, N.J.). The rPERKmyc-2cut, was then generated by cloning mPERK(450 bp)-myc from pcDNA-mPERK-9E10 (in house) into rPERK-2cut through SapI and XhoI to replace rPERK(450 bp). The 150aa C-terminus is conserved between mPERK and rPERK. The EGFP-2cut for mIns2 targeting was generated by cloning ITR-U6-mINS2utr5sg-5pCUT-EGFP-CDS-pA-3pCUT-ITR into pUC57-Kan through EcoRV (synthesized by GenScript). A short (49 bp) polyadenylation signal was used as previous described (Suzuki et al., Nature, 540:144-149 (2016), which is hereby incorporated by reference in its entirety). AAV-U6-mINS2utr5sg-EGFP-2cut in serotype 8 or DJ was packaged using EGFP-2cut. CopGFP-CDS-SV40 pA sequence were obtained from Lonza of its pmaxGFP plasmid. The CopGFP-2cut for hINS targeting was generated by cloning ITR-U6-BbsI-scaffold-hINSin1 (flipped cut site for sg-Reverse)-CopGFP-CDS-SV40 pA-3×3pCUT-ITR into pUC57-Kan through EcoRV (synthesized by GenScript). The CopGFP-1cut was generated by MfeI double digestion to remove the 3×3pCUT from the CopGFP-2cut. The CopGFP-1cut (or 2cut) with U6-hINSin1sg was constructed by cloning the hINSin1sg-Reverse into BbsI site and was then used either in plasmid experiment or to package AAV-DJ-U6-hINSin1sg-CopGFP-1cut (or 2cut). pAAV-nEF-Cas9 was purchased from Addgene (plasmid #87115, deposited by Juan Belmonte) and was used either in plasmid experiment or AAV-nEF-Cas9 packaging in serotype DJ.

Cell culture—Mouse embryonic fibroblasts (MEF) cells were immortalized from Perk^{Δex7-9/Δex7-9} (Jiang, et al., Mol. Cell Biol., 23:5651-5663 (2003), which is hereby incorporated by reference in its entirety) and Perk^{C528X/C528X} mice using a plasmid carrying the SV40 large T antigen (SV40 1: pBSSVD2005, Addgene, plasmid #21826, deposited by David Ron). Following immortalization, MEF cells were maintained in Dulbecco's Modified Eagle Medium, DMEM (Gibco, Gaithersburg, Md.) supplemented with 10% fetal bovine serum, FBS (Gemini, West Sacramento, Calif.) and 1× Penicillin-Streptomycin (Pen-Strep) at 100 U/mL-100 μg/mL (Gibco). Mouse MIN6 (Dr. Jun-Ichi Miyazaki, Osaka University, Japan) beta cells and human AD293 cells (Agilent, Santa Clara, Calif.) were cultured under the same conditions as MEF cells. Primary human cadaveric islets were obtained from Prodo Labs of Integrated Islet Distribution Program (IIDP). Upon receipt, islets were transferred from shipping media to CMRL 1066 (Connaught Medical Research Laboratories, Toronto, Canada; purchased from Gibco) supplemented with 10% FBS, 1× Pen-Strep and 2 mM L-Glutamine (Gibco) at a concentration of 800-1000 islet equivalents (IEQ) per milliliter in a non-tissue culture treated 6 cm dish and cultured overnight. All cells were cultured in a humidified, 5% CO₂ incubator at 37° C.

Plasmid transfection via electroporation—Perk^{Δex7-9/Δex7-9} MEF cells were transfected with CRISPR/Cas9 and CRBR donor constructs by electroporation using the MEF 2 Nucleofector Kit (Lonza, Basel, Switzerland), program T-20 in Nucleofector™ 2b Device (Lonza) according to the manufacturer's protocol. MIN6 cells were similarly electroporated using Nucleofector Kit V (Lonza), program G-16. The pmaxGFP plasmid provided in the Nucleofector Kit was used as transfection positive control in all plasmid electroporation experiments. To achieve higher electroporation efficiency, the Neon Transfection system (Invitrogen) was used for the following cells in a 10 μL electroporation system (Invitrogen) with no more than 1 μg plasmid DNA per 10 μL treatment: Perk^{C528X/C528X} MEF cells, 1×10⁷cells/mL, 1650V, 20 ms, 1 pulse; AD293 cells, 5×10⁶cells/mL, 1245V, 10 ms, 3 pulses; human islets, 500 IEQs/10 μL, 1050V, 40 ms, 1 pulse. The Neon procedure for electroporation of human islets was adapted from previously described protocols (Tamaki et al., BMC Biotechnol., 14:86 (2014) and Lefebvre et al., BMC Biotechnol., 10:28 (2010), both of which are hereby incorporated by reference in their entirety), Briefly, about 1000 IEQs for two replicates of one treatment was transferred to a 1.5 mL tube and centrifuged for 1 min at 100 g and washed with PBS and re-centrifuged. The islets were then incubated with Accutase (Gibco) for 2 min at 37° C. to partially dissociate them, and then washed with PBS and resuspended in 20 μL R buffer with 2 μg of each plasmid DNA needed for the treatment. About 500 IEQs in 10 μL with 1 μg plasmid DNA were electroporated with 1 pulse at 1050V for 40 ms and then cultured individually in a non-tissue culture treated 24-well plate.

AAV production and titration—AAVs carrying hGFAP::Cre and CAG::FLEx-GFP for serotype testing in human islets were as previously described. Chen et al., Mol. Ther., 28:217-234 (2020), which is hereby incorporated by reference in its entirety. AAV8-U6-mINS2utr5sg-EGFP-2cut (6.15×10¹³GC/mL) was produced and purified by Penn Vector Core. AAV-DJ-U6-mINS2utr5sg-EGFP-2cut (2.92×10¹²GC/mL), AAV-DJ-U6-hINSin1sg-CopGFP-2cut (1.83×10¹³GC/mL), AAV-DJ-U6-hINSin1sg-CopGFP-1cut (6.02×10¹²GC/mL), and AAV-DJ-nEF-Cas9 (3.83×10¹²GC/mL) were produced and purified as described below. Briefly, recombinant AAVs were produced in 293AAV cells (Cell Biolabs, San Diego, Calif.). Polyethylenimine (PEI, linear, MW 25,000) was used for transfection of three plasmids: the pAAV vector constructs, pAAV2/8-RC (Penn Vector Core) or pAAV-DJ (Cell Biolabs) and pHelper (Cell Biolabs). At 72 hours post-transfection, cells were scrapped in their medium, centrifuged, and then frozen and thawed four times by placing it alternately in dry ice-ethanol and a 37° C. water bath to lyse the cells and release the virus. The resulting AAV crude lysate was purified by centrifugation at 54,000 rpm for 1 hr in discontinuous iodixanol gradients with a Beckman SW55Ti rotor. The virus-containing layer was extracted, and viruses were concentrated by Millipore Amicon Ultra Centrifugal Filters (Millipore-Sigma, Bedford Mass.). Virus titers were determined by qPCR according to Addgene protocol.

AAV transduction of human islets—AAV-DJ-U6-hINSin1sg-CopGFP-2cut, AAV-DJ-U6-hINSin1sg-CopGFP-1cut and AAV-DJ-nEF-Cas9 were added to 300 IEQs cultured overnight in 200 μL CMRL1066 medium with reduce FBS (2%) at a final titer of 9.0×10¹⁰ GC/mL. If 1 IEQ is considered to be 1000 cells, the AAV incubation of human islets was at 60,000 MOI. CMRL1066 medium with 10% FBS was added to the sample at 1d post-infection.

AAV administration via intravenous injection—Two-week-old Cas9-EGFP mice were injected with 20 μL or 40 μL of AAV8-U6-mINS2utr5sg-EGFP-2cut, via retro-orbital (r.o.) injection. Eight-week-old Cas9-EGFP mice were injected with 50 μL of AAV8-U6-mINS2utr5sg-EGFP-2cut or AAV-DJ-U6-mINS2utr5sg-EGFP-2cut, or 504, saline solution via tail vein injection. Six-month-old C57BL/6J mice were injected with 100 μL of AAV-DJ mixture (50 μL of AAV8-U6-mINS2utr5sg-EGFP-2cut, with or without 50 μL of AAV-DJ-nEF-Cas9), or 100 μL saline solution via tail vein injection.

Single cell sorting—MEF cells and MIN6 cells were single cell sorted according to size configuration or GFP fluorescent signal using Beckman Coulter MoFlo Astrios (Beckman-Coulter, Brea, Calif.) performed by Flow Cytometry Facility at the Huck Institutes of the Life Sciences at Penn State University. Cells were dissociated using 0.25% Trypsin-EDTA solution for 5 min at 37° C. and warm DMEM medium supplemented with 10% FBS was added to stop trypsinization. Cells were then transferred into a 15 mL tube and centrifuged at 200 g for 1 min at room temperature. The cells were re-suspended thoroughly in DMEM medium with 1× Pen-Strep as single cells and were sorted into 96-well plate with full DMEM medium.

Genomic DNA extraction and diagnostic PCR analysis—Genomic DNA was extracted from cultured cells or mouse tissue by digesting in lysis buffer (5 mM EDTA, 0.2% SDS, 200 mM NaCl, and 100 mM Tris-HCl, pH8.5) with 100 μg/mL proteinase K overnight at 50° C. DNA was then precipitated with 1 volume of isopropanol and dissolved in TE buffer (10 mM Tris-HCl, 1.0 mM EDTA, pH8.0). Blood DNA was extracted using Monarch Genomic DNA Purification Kit (New England Biolabs). Diagnostic PCRs were performed using GoTaq Master Mix (Promega, Madison, Wis.). Five percent of DMSO was added to improve amplification of GC-rich sequences. PCR product purification was carried out using the QIAquick PCR Purification Kit (Qiagen, Hilden, Germany). Gel purification to recover PCR fragments after electrophoretic separation was performed using the Zymoclean Gel DNA Recovery Kit (Zymo, Irvine, Calif.). Sanger sequencing of the PCR products was performed by Genomics Core Facility at the Huck Institutes of the Life Sciences at Penn State University. DNA sequencing results were analyzed using the SnapGene software.

RNA isolation and quantitative PCR analysis—Total RNA from cell lines and mouse tissues other than pancreas was extracted using the Quick-RNA Miniprep Kit (Zymo). Pancreas RNA was extracted as previously described by Robert C. De Lisle (10.3998/panc.2014. 9). Human islet RNA was extracted using AliPrep DNA/RNA/Protein Mini Kit (Qiagen). Reverse transcription was performed using qScript cDNA SuperMix (Quanta, Beverly, Mass.). Quantitative mRNA measurement was carried out using PerfeCTa SYBR Green SuperMix ROX (Quanta) with the StepOnePlus Real-time PCR system (Applied Biosystems, Foster City, Calif.). Gene expression levels were normalized to endogenous mouse Actin (Actb) or human Actin (ACTA1) levels of the same sample. The relative fold change in expression was calculated using the ΔΔCt method.

Digital droplet PCR—Quantification of CRBR editing efficiency at genomic DNA level was performed by digital droplet PCR (Hindson et al., Anal. Chem., 83:8604-8610 (2011) and Tomaszkiewicz et al., Genome Res., 26:530-540 (2016), both of which are hereby incorporated by reference in their entirety) using a QX200 ddPCR system (Bio-Rad, Hercules, Calif.). The ddPCR reaction contained final concentrations of the following components: 1× EvaGreen Supermix (Bio-Rad), 150 nM of each primer, 0.13U/4, of HindIII-HF (New England Biolabs), and template DNA (human AD293 cell or human islet DNA, 5Ong/reaction; mouse tissue DNA, 200 ng/reaction). Formation of droplet emulsions was performed by mixing 20 μL of PCR reaction and 70 μL of EvaGreen droplet generation oil (Bio-Rad) with the Automatic Droplet Generator (Bio-Rad) and was dispensed into 96-well plate. The emulsions containing approximately 20,000 droplets were cycled to amplicon saturation using a C1000 Thermal Cycler (Bio-Rad) operating at the following conditions: for 5 min at 95° C., 40 cycles of 30 sec at 94° C. and for 1 min at 59-63.3° C. (optimized for each primer set), for 5 min at 4° C., for 5 min at 90° C., and a 4° C. hold. Amplitude of fluorescence by amplicons in each cycled droplet was measured using flow cytometry on a QX200 Droplet Reader (Bio-Rad) set on the EVA channel The QuantaSoft droplet reader software (v1.4.0.99; Bio-Rad) was used to cluster droplets into distinct positive and negative fluorescent groups and fit the fraction of positive droplets to a Poisson algorithm to determine the starting concentration (copies/μL) of the input DNA sample. CRBR editing efficiency was calculated by the ratio of the 5′ junction concentration (including clean CRBR integration and 5′ CRBR whole donor integration) to the reference gene concentration. The reference genes in mouse and human genome, mRpp30 (chr19) or hRPP30 (chr10), have the same copy number as the chromosomal alleles to be edited, mouse Ins2 locus on chr7, or human INS locus on chr11.

GFP imaging and histological analysis—MIN6 cells and human islets were imaged as live cultures and images were captured using the FITC and Transillumination channels of the ECHO Revolve microscope and the associated software (Echo Labs, San Diego, Calif.). Whole pancreata were harvested and paraffin embedded as previously described in Zhang et al., Molecular and Cellular Biology, 22:3864-3874 (2002), which is hereby incorporated by reference in its entirety). Sectioned (6 μm in thickness) slides were dewaxed, and Hematoxylin and Eosin stained by Leica Autostainer ST5010 XL (Wetzlar, Germany). Bright field images were captured with the ECHO Revolve microscope.

Immunoblot analysis—Total cell lysates were made from mouse pancreatic tissue using RIPA buffer (1% Nonidet P40, 0.5% sodium doxycholate, 0.1% SDS, 1×PBS, pH 8.0) with 1× Protease Inhibitor cocktails and 1× Phosphatase Inhibitor cocktail 2 and 3 (Millipore-Sigma). Lysate proteins from tissues or MEF cells were denatured by boiling the lysates in 2x SDS sample buffer for 5 min prior to electrophoresis on NuPAGE 8% Bis-Tris Midi gel (Invitrogen). The separated proteins were transferred to nitrocellulose membranes (0.45 μm, Thermo Scientific, Waltham, Mass.) in carbonate transfer buffer using wet transfer conditions (Criterion Blotter, Bio-Rad). Primary antibodies (diluted in 5% BSA-TBST) used include: Phospho-PERK (Thr980) (#3179, Cell signaling, Danvers, Mass.), PERK (#3192, Cell Signaling), Phospho-eIF2α (Ser51) (#9721, Cell signaling), eIF2α (#AHO1182, Invitrogen), Myc Tag (#R950-25, Invitrogen) and Actin (#A5060, Millipore-Sigma). Appropriate IRDye-conjugated secondary antibodies were used, and IR fluorescence was detected using the LI-COR Odyssey CLx Imaging System and quantified using the LI-COR Image Studio Software (LI-COR, Lincoln, Nebr.).

Statistical analysis—Numerical data were represented as mean+/−SE. Statistical significance was determined using Student's t-test, where appropriate.

### Example 1—CRBR-Mediated In Vitro PERK CDS Integration in Perk KO Cell Line

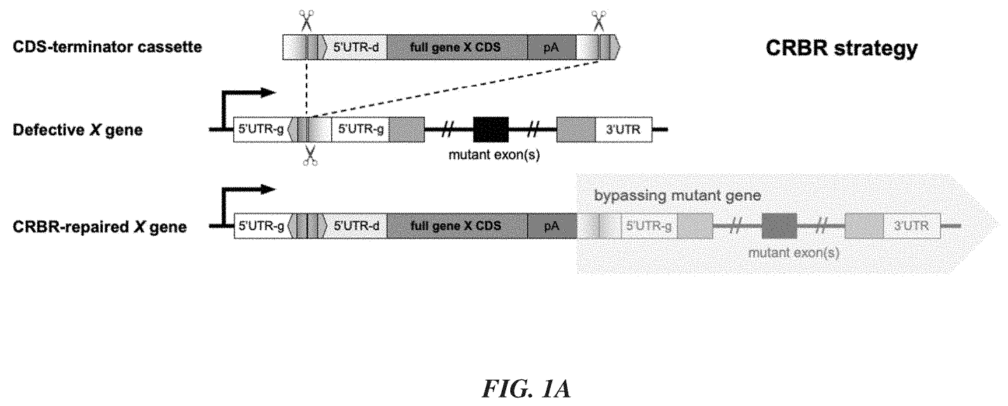

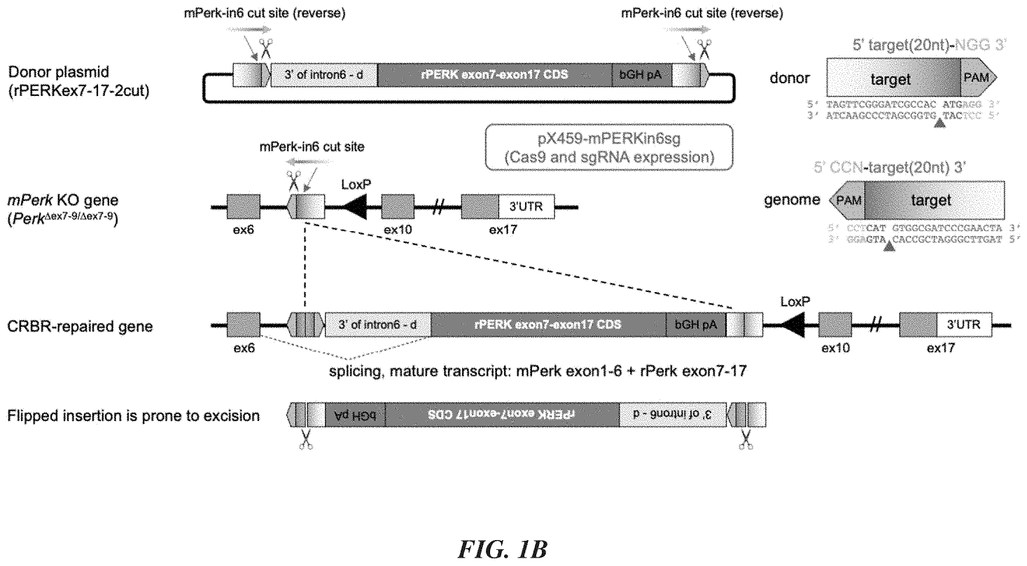

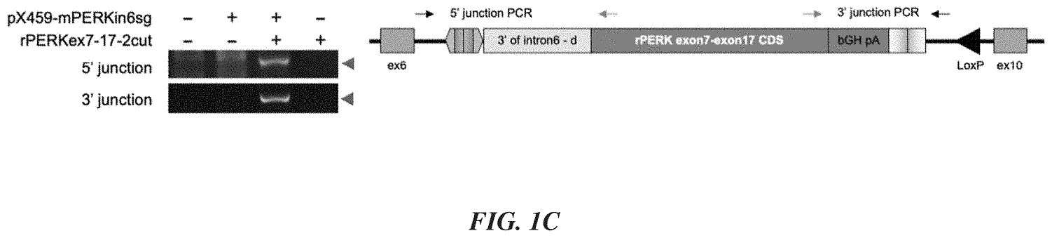

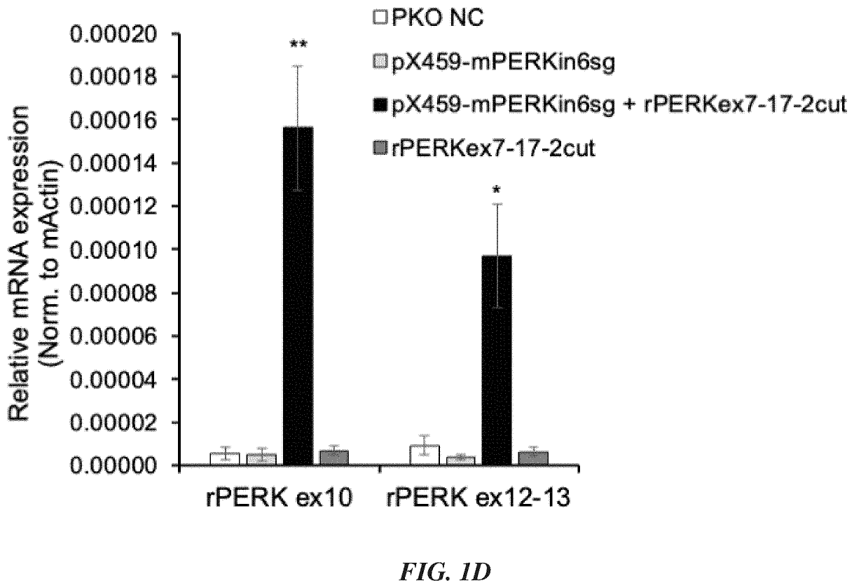

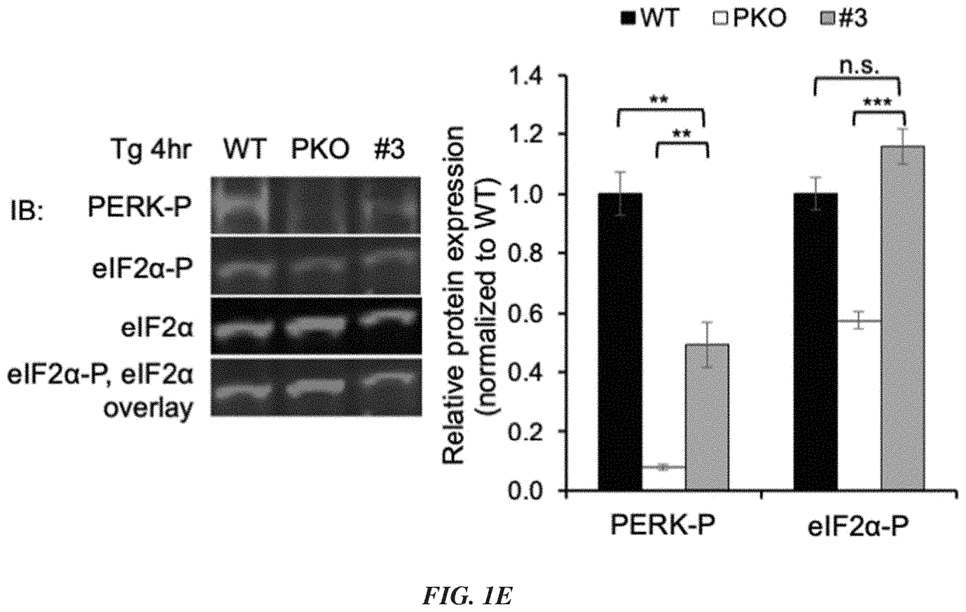

The CRBR strategy features a genome editing process that generates a Cas9/sgRNA targeted DSB at a non-coding region in the genome, either within the 5′UTR or an intron. The same Cas9/sgRNA cut sites are engineered in the donor to promote the insertion of a wild-type coding sequence with transcription termination into the genomic DSB (FIG. 1A). The CRBR-edited allele expresses the inserted CDS-terminator cassette under control of the endogenous promoter and bypasses expression of the downstream mutation.

The CRBR strategy was first tested in a Perk KO mouse embryonic fibroblast (MEF) cell line (Perk^{Δex7-9/Δex7-9}) in which exons 7-9 have been deleted. A partial CDS (˜2.2kb) containing the 3′ end of intron 6 and exons 7-17 of rat Perk followed by a heterologous polyadenylation signal (bGHpA) was designed to integrate into the endogenous intron 6 to restore normal PERK expression. The Perk gene is highly conserved in rodents and the rat Perk gene has previously been shown to be fully functional in mice (Zhang et al., Molecular and Cellular Biology, 22:3864-3874 (2002), which is hereby incorporated by reference in its entirety), therefore, using the rat Perk CDS was advantageous for distinguishing between endogenous mouse Perk and the CRBR integrated rat Perk. A Cas9/sgRNA target cut site identified within intron 6 was engineered into the donor plasmid with reverse orientation flanking the 3′in6-rPERKex7to17-bGHpA cassette (FIG. 1B). The rPERKex7-17-2cut CRBR cassette can be integrated in two possible orientations: the correct 5′-5′/3′-3′ orientation and the incorrect, “flipped” 5′-3′/5′-3′ orientation. The cassette cut sites were designed in reversed orientation so that the correctly oriented integrants would not regenerate the cut sites whereas the incorrectly oriented integrants would restore them. Consequently, incorrectly oriented integrants could be re-excised by Cas9 for possible re-insertion in the correct orientation. Perk KO MEF cells co-transfected with the Cas9/sgRNA plasmid and the rPERKex7-17-2cut plasmid were positive for the 5′ and 3′ junction diagnostic PCRs (FIG. 1C), indicating the presence of correctly edited cells within the population. The chimeric mouse-rat Perk mRNA was also detected in this mixed cell population (FIG. 1D).

This mixed population was then sorted into single cells and expanded to create 96 independent cell lines with two possible Perk alleles. Among the 96 single sorted cell lines, thirty-three cell lines were positive for the 5′ junction diagnostic PCR. In order to test for functional PERK restoration in the CRBR-edited Perk KO MEF cells, eight cell lines were chosen and subjected to thapsigargin treatment, which induces ER stress by PERK auto-phosphorylation and phosphorylation of its major substrate eIF2a. Cell line #3 had detectable levels of both PERK-P and eIF2α-P, indicating that a functional chimeric PERK protein was expressed in this cell line (FIG. 1E). CRBR-editing was confirmed in seven other single sorted cell lines at the genome level, but PERK protein expression could not be detected in these lines. In these cases, it is suspected that the 5′ junction within the intron 6 of CRBR-edited Perk altered the splicing signal between the mouse exon 6 and rat exon 7-17 CDS of the cassette. Cell line #3, which expressed functional PERK, had an 11 bp deletion at the 5′ junction that removed an unintended cryptic splice-acceptor site (AG/G), which fortuitously reversed the splicing defect. The 5′ junction of the other 7 non-expressing cell lines occurred as designed (either a clean joint or 1-2 bp indels) but retained the splice-acceptor. The resulting alternative mature transcript in these non-expressing cell lines contained an extra 135 bp intronic sequence that encoded a stop codon, which likely resulted in nonsense-mediated mRNA decay (NMD). These results show that a CRBR-mediated partial-CDS gene editing can restore Perk gene expression and gene function in Perk KO cell line, but the introduction of cryptic splice sites needs to be avoided.

### Example 2—rPERK-CRBR-Edited Perk Allele Completely Rescues Perk KO Mice

To circumvent the RNA splicing defects that might be generated during NHEJ-DSB repair at the 5′ junction, the CRBR strategy was modified so that an entire, fully-spliced rat PERK CDS carrying a c-terminal myc tag was targeted to the 5′UTR of the mouse Perk gene. The rPERKmyc-2cut CRBR cassette consists of the intact mouse Perk 5′UTR, a rPERK CDS (˜3.4 kb) with a myc tag, a bGHpA terminator, and a Cas9/sgRNA target site engineered in reverse orientation (FIG. 2A). This modified CRBR strategy preserves the sequence of the mouse Perk 5′UTR to ensure normal translation initiation. The Perk KO nonsense mutant MEF cell line (Perk^{C528X/C528X}) co-transfected with the Cas9/sgRNA plasmid and the rPERKmyc-2cut plasmid was positive for both 5′ and 3′ junction diagnostic PCRs (FIG. 2B), which confirmed the CRBR-Full-CDS integration at the intended target site in the genome in vitro.

To demonstrate that the CRBR-edited allele can be expressed and regulated normally at the mRNA and protein level during development, an in vivo proof-of-concept experiment was designed to test if an engineered rPERK-CRBR-edited allele could rescue a Perk KO allele in mice. A key assumption of this strategy is that the integration of the CRBR cassette into a wild type Perk allele will generate a complete loss-of-function insertional mutation of the endogenous allele while simultaneously introducing a functional CRBR cassette under the endogenous promoter. The CRBR cassette-insertional mutation can be genetically crossed to a mouse bearing any other type of Perk null mutation to generate offspring that carry the CRBR cassette-insertional mutation on one chromosome and a Perk null mutation on the other. If these mice express PERK only from the correctly targeted CRBR cassette and are phenotypically normal with respect to the WRS phenotype, the ability of CRBR to rescue PERK expression and function in vivo would be confirmed.

The SpCas9 protein, mPERK-utr5-sgRNA, and the rPERKmyc-2cut plasmid were microinjected into zygotes to create transgenic mice with the rPERKmyc-CDS integrated into the 5′UTR of the wild-type mouse Perk allele. Out of the 21 transgenic mice generated, one was positive for both 5′ and 3′ junction diagnostic PCRs. Further genotyping of F1 offspring from this founder mouse crossed to a wild-type mouse revealed the founder to be mosaic at the Perk locus (WT/4bpDel/rPERK-CRBR/flipped-backbone-CRBR), with the rPERK-CRBR allele having small indels in the 5′UTR region (FIG. 3A). The F1 Perk^{+/rPERK-CRBR} mice were then crossed to mice heterozygous for a Perk null allele (Perk^{C528X/+} or Perk^{Δex7-9/+}). Some of these F2 offspring were genotyped to be KO/rPERK-CRBR heterozygotes (Perk^{C528X/rPERK-CRBR} or Perk^{Δex7-9/rPERK-CRBR}), healthy and fertile. Perk KO mice exhibit high neonatal lethality (50-99%), and those mice that survive exhibit severe growth retardation, low pancreatic beta cell mass, exocrine pancreas atrophy, and extreme hyperglycemia by four weeks of age (Zhang et al., Molecular and Cellular Biology, 22:3864-3874 (2002); Zhang et al., Cell Metabolism, 4:491-497 (2006); Li et al., Endocrinology, 144:3505-3513 (2003); and Iida et al., BMC Cell Biology, 8:38 (2007), all of which are hereby incorporated by reference in their entirety). The rPERK-CRBR allele showed complete phenotypic rescue of both the Perk nonsense null mutant (FIGS. 3B-3C) and the Perk Δex7-9 deletion mutant with respect to survivorship, growth, beta cell mass, exocrine pancreas viability, and glucose homeostasis.

Perk mRNA levels from both the rPERK-CRBR cassette and the endogenous mouse Perk were analyzed to determine if the CRBR-integrated rPerk was expressed and if the CRBR insertion blocked expression of the downstream mPerk mRNA as expected from the experimental design. The rPERK-CRBR cassette was robustly expressed in the pancreas and brain in genotypes carrying one or two rPERK-CRBR alleles and was absent in mice lacking the rPERK-CRBR cassette (FIG. 3D). Similarly, mPerk expression was seen in genotypes carrying one or two copies of the wild-type mouse Perk allele, with reduced expression in genotypes carrying the C528X nonsense mutation. The reduction of mouse Perk mRNA in the latter is likely caused by NMD. The insertion of the CRBR cassette into the wild-type mouse allele resulted in a ˜95% reduction in mouse Perk mRNA. Therefore, it is estimated that ˜5% of the primary transcripts in the CRBR alleles are transcriptional read-through of the rPERKmyc-bGHpA terminator within the CRBR cassette resulting in low-level of the downstream mouse Perk mRNA transcript. This small fraction of transcripts generated by failure to terminate at the bGH polyA terminator are bicistronic, comprised of rPERK-myc followed by mPERK. It is very unlikely that the mPERK sequences within this hybrid CDS would be translated, because normal cap-dependent translation initiates only at the first CDS which, in this case, is the rPERK-myc CDS. Any translation of the downstream mPERK CDS would require that the 40S ribosome either remain on the mRNA after translation termination of the rPERK-myc CDS with subsequent translation re-initiation or bind internally upstream of mPERK CDS in a cap-independent mechanism (Hellen et al., Genes Dev., 15:1593-1612 (2001), which is hereby incorporated by reference in its entirety). Both of these possibilities are highly unlikely as they require specialized sequence contexts (Gunisova et al., FEMS Microbiol. Rev., 42:165-192 (2018), which is hereby incorporated by reference in its entirety) that are absent in this case. Consequently, a low level of transcriptional read through in a CRBR engineered gene correction scheme should not interfere with the CRBR strategy to bypass translation of the downstream endogenous coding sequence.

Consistent with their phenotypic rescue, the C528X/CRBR and Δex7-9/CRBR mice expressed a substantial level of rPerk mRNA derived from the CRBR cassette. Low-level detection of mPerk mRNA in these mice was contributed by the KO mutant allele and by the CRBR allele (leaky transcriptional read-through), neither of which are competent for normal translation. It is concluded, therefore, that the CRBR rescue of Perk null mutations is due solely to the expression of the rPERK protein translated from the rPERK-CRBR cassette. Cassette-derived rPERK protein expression was confirmed by immunoblotting with a myc antibody as well as an antibody that recognizes both rat and mouse PERK (FIG. 3E). Critically, the cassette-encoded myc-tagged rPERK showed strong expression in all genotypes bearing a rPERK-CRBR allele but not in other genotypes. Altogether, these results demonstrate that a CRBR-edited allele can rescue a null allele in a living organism. Additionally, they suggest the expression of the CDS-terminator cassette in a CRBR-repaired cell can be regulated normally under the endogenous promoter and provide therapeutic effects in vivo.

### Example 3—CRBR-Mediated In Vitro and In Vivo Gene Editing in Mouse Pancreatic Beta Cells

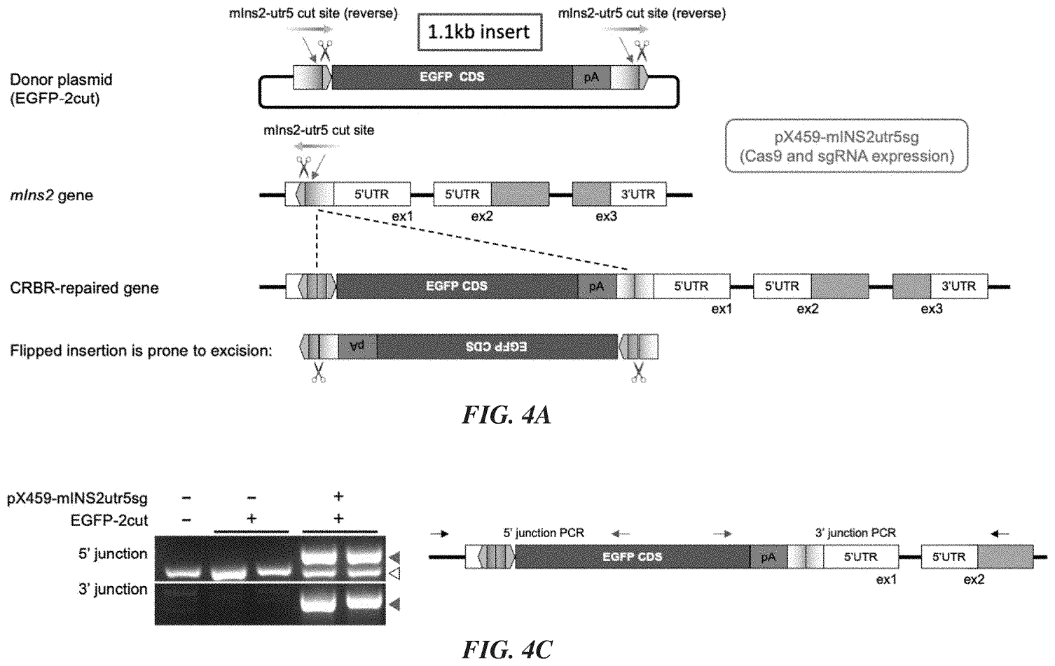

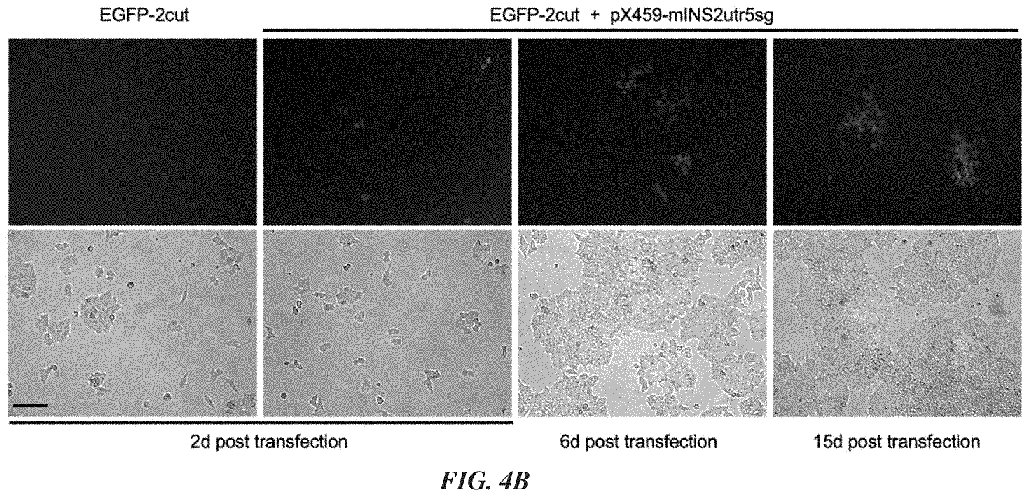

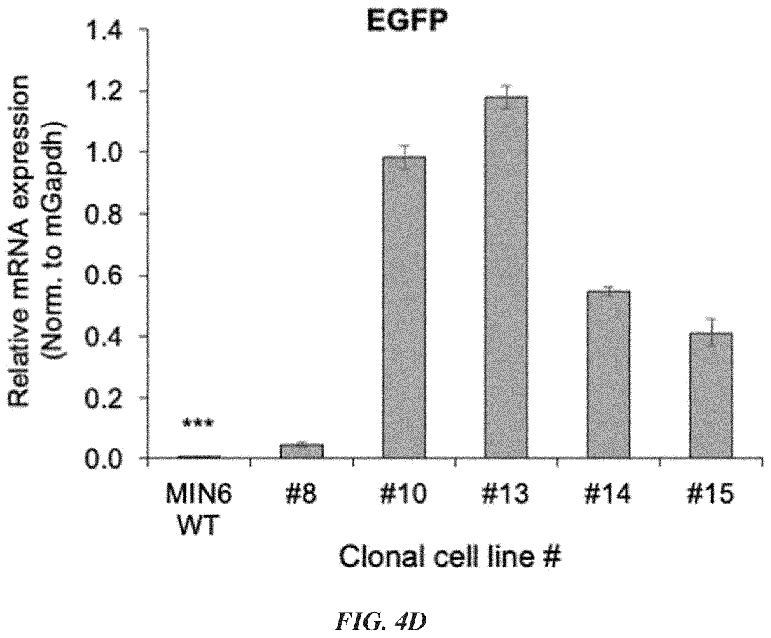

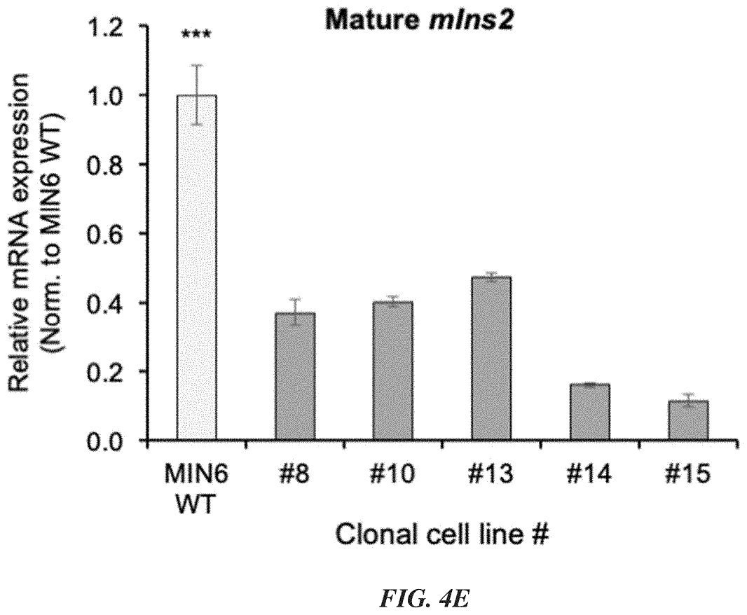

To more directly assess and visualize the protein expression from a CRBR-edited allele, a similar two-cut CRBR strategy was applied to introduce a GFP CDS into the Insulin gene locus, the most highly expressed gene within pancreatic beta cells. The Cas9/sgRNA cut sites were designed in the reverse orientation relative to the native cut site in the 5′UTR target site of the mouse Ins2 gene (FIG. 4A) to increase the likelihood that the EGFP-CDS-pA cassette (˜1.1 kb) remains stably integrated. This design feature, however, did alter the 5′UTR from the wild-type sequence with small changes resulted from the residue target site in the donor. To avoid potential interference with translation, an integration site was selected within a region that is not conserved among mammals, and the introduction of new ATG codons within the CRBR-edited 5′UTR was avoided that could incorrectly initiate translation of the resulting mRNA. This strategy was first tested in MIN6 mouse beta cells by co-transfecting them with the Cas9/sgRNA plasmid and the EGFP-2cut donor plasmid. EGFP-positive cells were visible by 2-day post-transfection and continued to increase in number through 15 days, whereas donor-only treated cells remained EGFP-negative over the same time period (FIG. 4B). 5′ and 3′ junction analyses of the integrants confirmed CRBR-editing at the genome level (FIG. 4C). Single cell sorting revealed that the mixed population contained 2.5% GFP-positive cells; the low percentage of positive cells reflects the relatively poor transfection efficiency of MIN6 cells (˜25%).

A subset of GFP-positive cells was clonally isolated for further characterization. Junction PCRs and DNA sequence analyses showed that cell lines #8, #10, #13, and #14 had one CRBR-edited allele and one allele with small indels at the genomic cleavage site. The cell line #15 had one CRBR-edited allele and one whole donor plasmid integrated allele. EGFP mRNA expression was confirmed in the sorted GFP-positive cell lines (FIG. 4D). It was also expected that the native mouse Ins2 expression would be reduced as a consequence of the insertion of the EGFP CRBR cassette. Indeed, it was found that the mouse Ins2 mRNA levels were reduced compared to wild-type MIN6 cells (FIG. 4E). These results suggest that the CRBR-integrated EGFP-CDS-pA cassette is expressed and can bypass the endogenous mouse Ins2 transcription.

To evaluate the capability of CRBR-mediated gene editing in the mouse pancreas in vivo, an AAV carrying the EGFP-CDS-pA cassette and U6-driven mINS2-utr5 sgRNA cassette (AAV-sgRNA-CDS) was systemically delivered to the Rosa26-CAG-Cas9-EGFP mouse strain, which constitutively expresses Cas9 nuclease throughout the body. Using a Cas9 expressing mouse strain substantially reduces the variability when compared to Cas9 delivery in trans via an additional viral vector. For comparison, the same AAV-sgRNA-CDS was also delivered into wild-type mice in combination with another AAV that does supply Cas9 in trans (AAV vectors, FIG. 5A). Liver and pancreas tissues from Cas9-EGFP mice were isolated 30-day post retro-orbital (r.o.) injection of the AAV8-sgRNA-CDS vector. Junction PCRs and ddPCR quantitation revealed substantial CRBR-mediated gene editing at the genome level in the liver (4.16% of chromosome 7 edited with CRBR integration of EGFP CDS) and a detectable level (0.64%) in the pancreas (FIG. 5B). Some individuals had detectable EGFP transcription from the mouse Ins2 gene locus in the pancreas RNA (FIG. 5C). The mouse Ins2 promoter is not active in the liver, therefore, and as expected, EGFP transcription from the Ins2 gene locus in the liver was not observed.

Previous experiments (Cheng et al., J. Biomed. Sci., 14:585-594 (2007); Rehman et al., Mol. Ther., 16:1409-1416 (2008); Mulder et al., J. Endocrinol., 240:123-132 (2019); and Grimm et al., J. Virol., 82:5887-5911 (2008), all of which are hereby incorporated by reference in their entirety) suggested that AAV serotypes DJ and 8 would be the most appropriate for delivery into the pancreas. Eight-week-old Cas9-EGFP mice were subjected to tail vein injection of AAV-sgRNA-CDS of either serotype DJ or 8. Both serotypes had substantial CRBR-mediated gene editing at the genome level in the liver, with AAV-DJ (8.39%) being more efficient (FIG. 5D). The tail vein injected AAV8-sgRNA-CDS was capable of targeting the pancreas (0.84%), with some individuals having detectable CRBR-editing at the genome level by junction PCRs. However, similarly administered AAV-DJ-sgRNA-CDS showed less pancreatic CRBR editing (0.29%). These results show that systemic delivery of the AAV8-CRBR-construct via intravenous injection can result in CRBR editing at the genome level in the liver and pancreas, as well as CRBR-mediated EGFP mRNA expression in pancreatic beta cells under the control of the Ins2 promoter.

It was next tested whether providing both the sgRNA-CDS and Cas9 via separate AAV-DJ vectors could also elicit gene editing in wild-type mice lacking endogenous Cas9. CRBR-mediated gene editing was achieved in the liver (FIG. 5E) by dual AAV administrations (0.56%), however, not with the same efficiency as was seen when Cas9 was endogenously expressed (FIG. 5D). Leaky expression of the promoterless EGFP CDS from AAV vector (ITR) was not observed in the liver of mice with AAV-DJ-sgRNA-CDS, although it is known that the ITR of AAV has weak promoter activity (Flotte et al., J. Biol. Chem., 268:3781-3790 (1993) and Haberman et al., J. Virol., 74:8732-8739 (2000), both of which are hereby incorporated by reference in their entirety). Overall, these results suggest that CRBR-mediated gene editing is feasible in vivo via dual AAV delivery once both viral vectors are successfully transduced in the host cell. Most importantly, the CRBR cassette expression is restricted to pancreatic beta cells under the insulin promoter.

### Example 4—CRBR-Mediated Ex Vivo Gene Editing in Human Islets

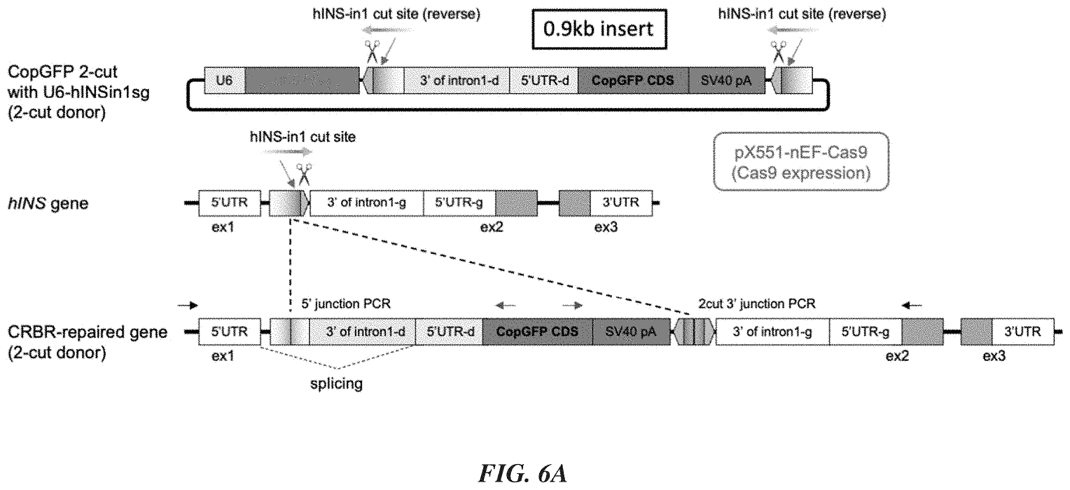

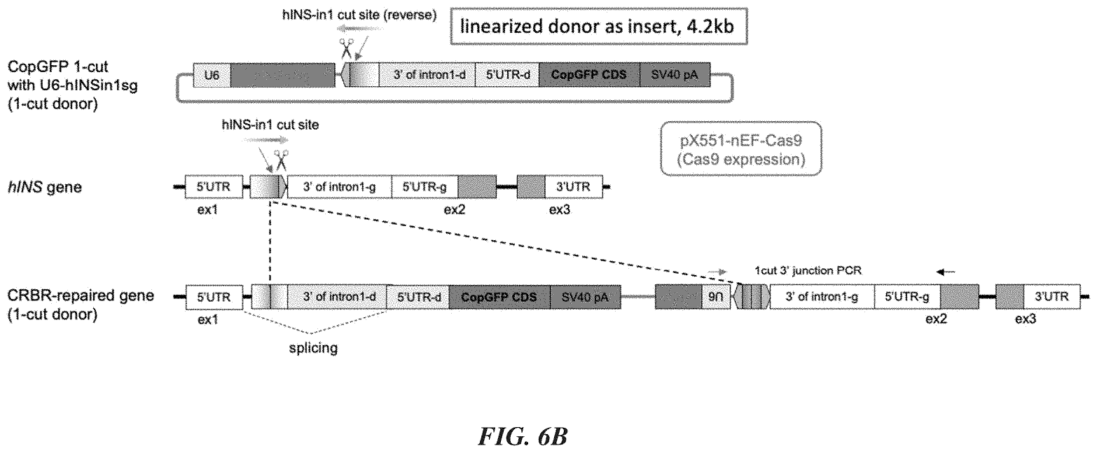

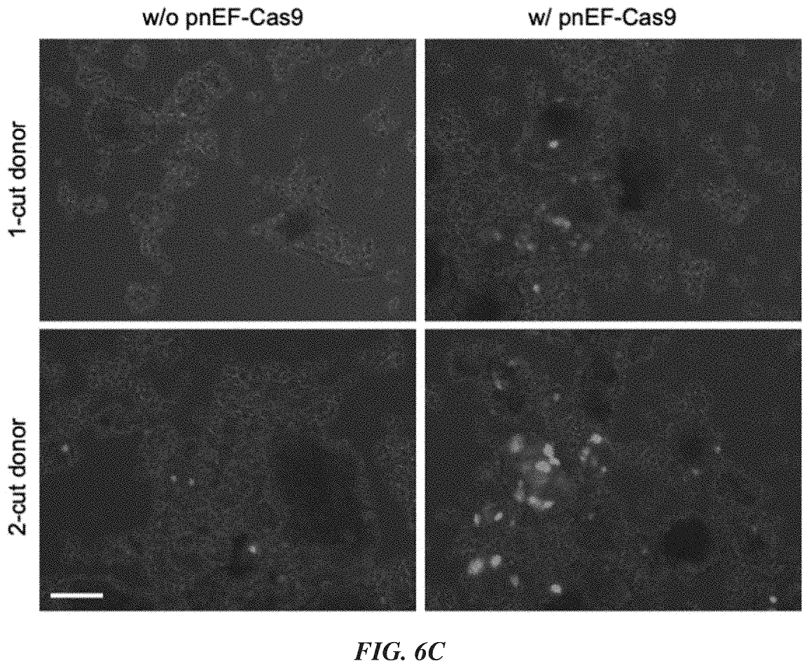

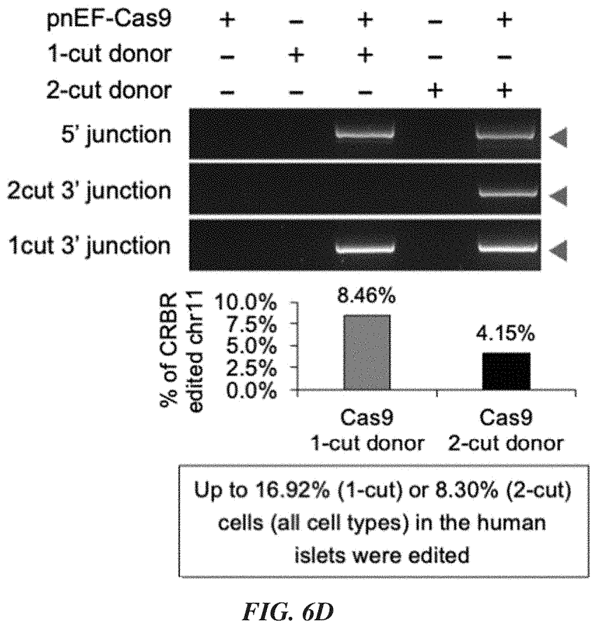

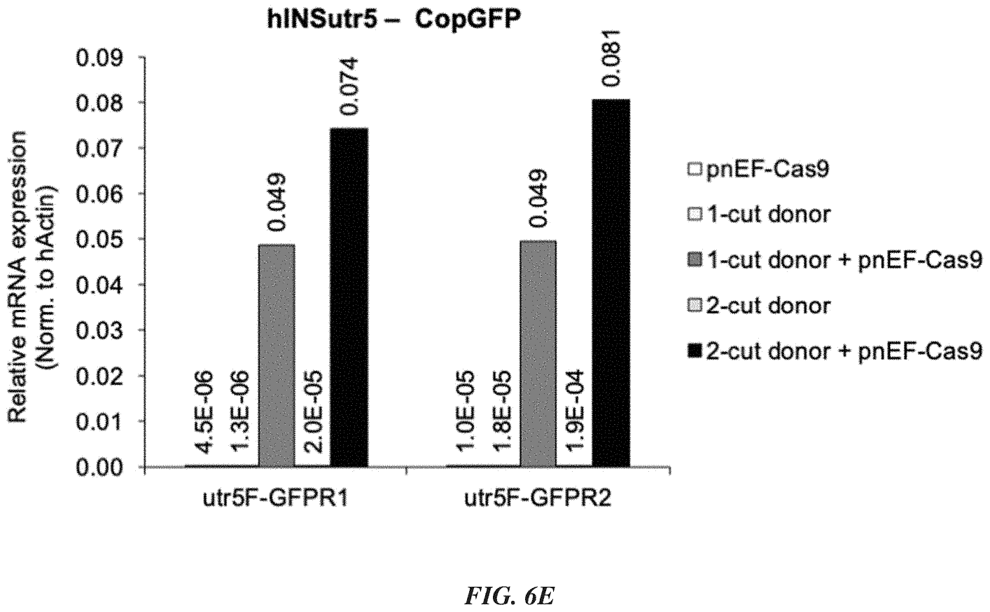

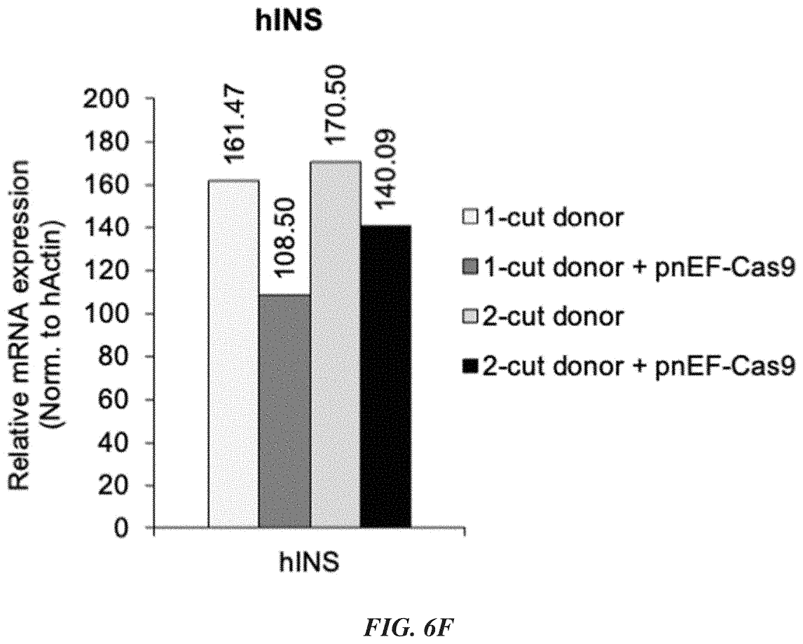

To further validate the CRBR strategy as a potential human gene therapeutic, GFP was similarly targeted to the insulin (INS) gene in isolated human islets. Primary human cadaveric islets were transfected or AAV infected with CRBR constructs containing CopGFP (alternative GFP reporter) CDS and targeting the INS gene. The CopGFP CRBR cassette was designed to insert into intron 1 between the two exons encoding the 5′UTR and upstream of the insulin start codon (FIG. 6A). The CRBR cassette contains sequences homologous to the 3′ half of the endogenous intron 1 as well as a region homologous to the 5′ UTR encoded by exon 2 which contains an acceptor splice site that is needed for proper splice excision of the newly integrated intron 1. By this design, any unforeseen indels generated during CRBR integration are spliced out of the resulting mature mRNA. In addition to the 2-cut donor, a 1-cut donor was introduced to determine which strategy was more efficacious (FIG. 6B). A one-cut strategy generates only one insert linearized from the 1-cut donor, with one correct integrant out of two possible outcomes (50%); whereas the two-cut strategy generates four possible inserts that may be integrated in two orientations, with two correct integrants out of eight possible outcomes (25%). For the one-cut strategy, a much larger fragment (4.2 kb) must be integrated. By contrast, the two-cut strategy integrates a much smaller fragment (0.9 kb, CRBR cassette only), as it excludes the extraneous vector sequences. However, these extraneous vector sequences should not interfere with gene expression because they are downstream of the transcription/translation terminators in the CRBR cassette.

This CRBR-CopGFP strategy was first tested in an easily transfected human cell line, AD293, to identify the optimal sgRNA target site within intron 1 and to optimize the donor design before testing in human islets. It was found that the reverse-oriented sgRNA (12.75%) outperformed the same-oriented sgRNA (4.56%) in CRBR integration. Six off-targets of the reverse-oriented sgRNA were then tested for possible off-target integration of the CopGFP CDS. Of these, three showed detectable off-target integrations (0.78-1.60%). Both the CopGFP 1-cut and 2-cut donor plasmids were engineered with a U6-hINSin1sg cassette which expresses the optimized reverse-oriented sgRNA. The SpCas9 expressing plasmid and the 1-cut or 2-cut donor plasmid were co-transfected into human islets. Six-day post-transfection, many CopGFP-positive islet cells were observed (FIG. 6C). This result indicates successful targeting to the pancreatic beta cells, as they are the only islet cell type with an active insulin promoter and comprise 45-70% of the total cadaver islet cell population. The remaining islet cells secrete other metabolically important peptide hormones (Da Silva Xavier, G., J. Clin. Med., 7(3):54 (2018), which is hereby incorporated by reference in its entirety). While these non-beta cell types should likely be edited with equal frequency compared to beta cells, their insulin promoter is inactive, and therefore would not be expected to express the CopGFP CRBR cassette. Junction PCRs confirmed CRBR editing of the human INS locus at the genome level (FIG. 6D), with 8.46% (1-cut) or 4.15% (2-cut) of chromosome 11 edited; and transcription of CopGFP from the human INS promoter was also detected (FIG. 6E). Furthermore, a modest reduction of human INS mRNA expression was observed (FIG. 6F), as expected. No biological replicates from the same batch of human islets were analyzed since the samples produce only enough genomic DNA or total RNA for one replicate per treatment. However, CopGFP integration at the genome level, CopGFP transcription, and reduction of human INS mRNA expression were seen in all human islet experiments using independent batches of islets. Collectively, these results demonstrate that CRBR-mediate gene correction via plasmid transfection is feasible in human islets if a wild-type coding sequence is targeted downstream of a mutant gene's promoter.

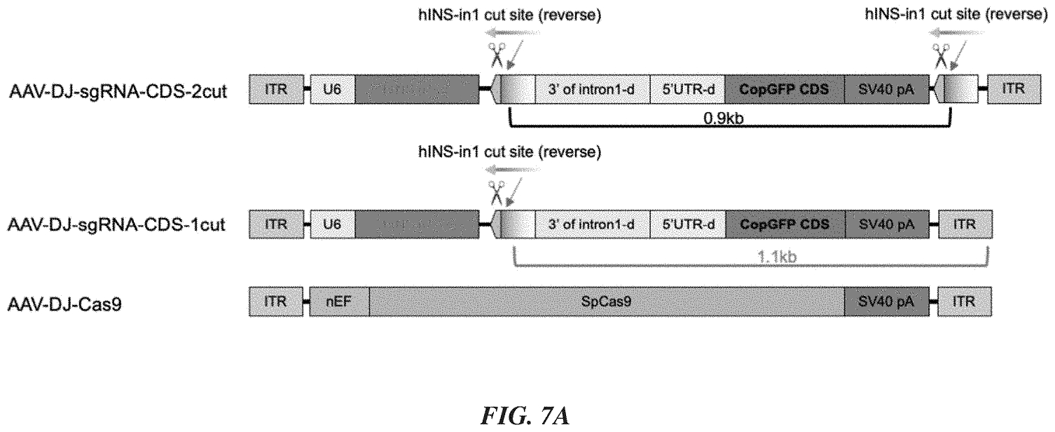

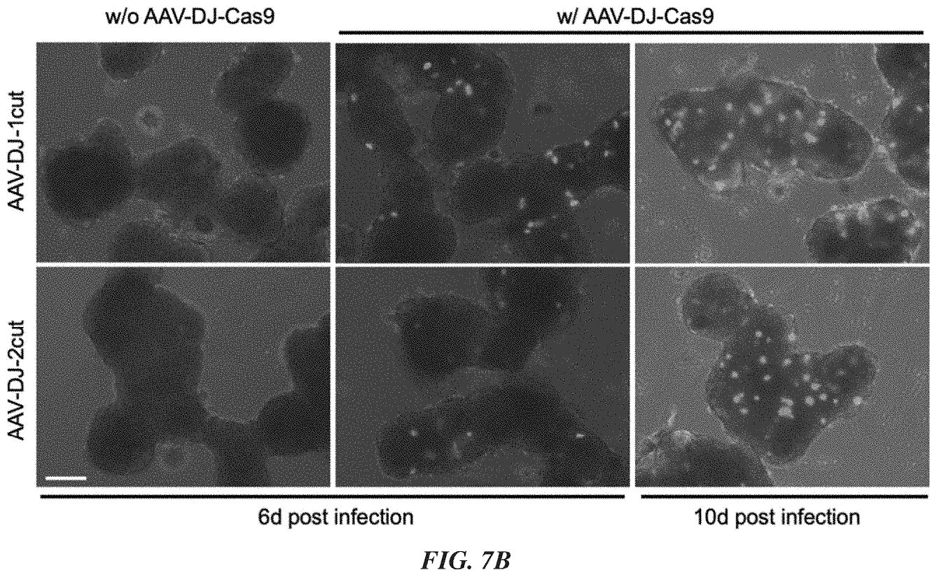

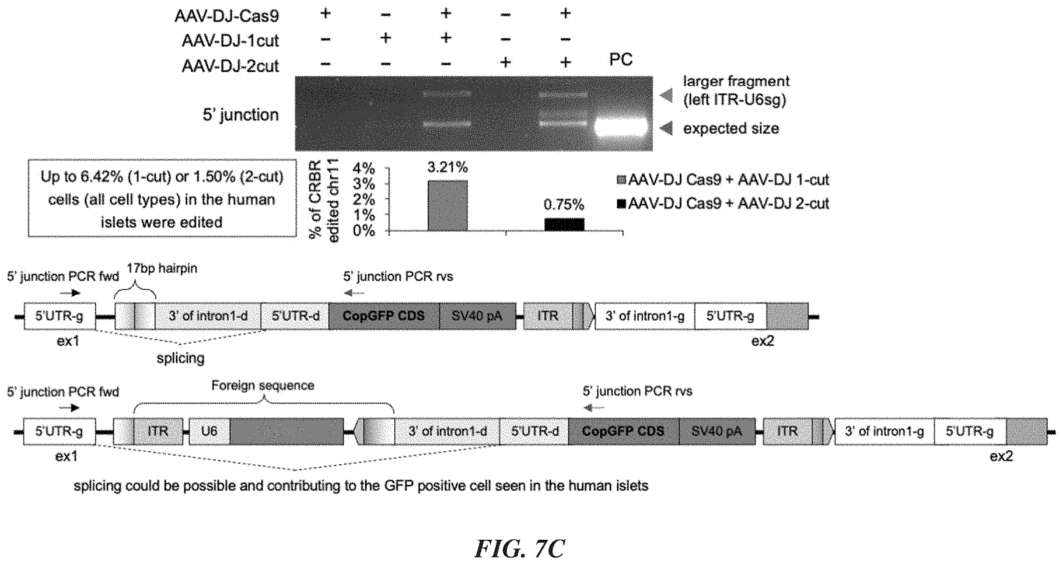

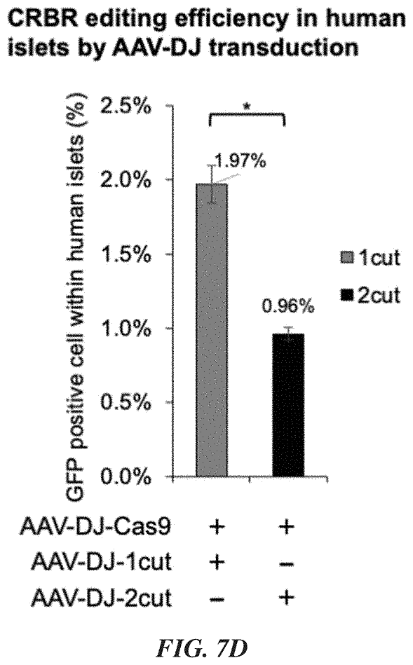

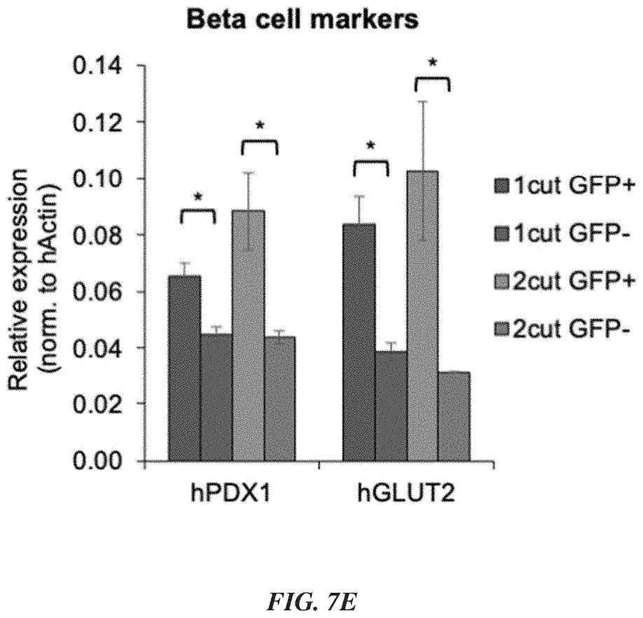

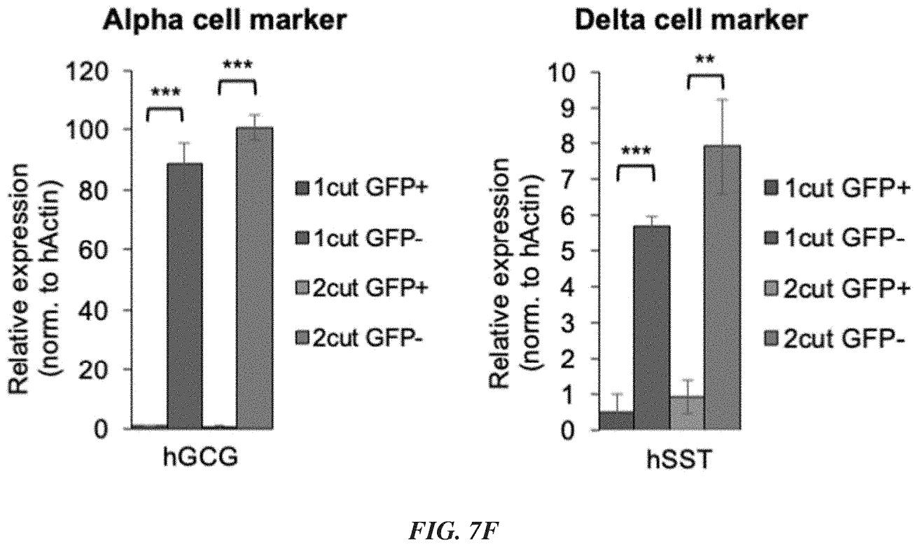

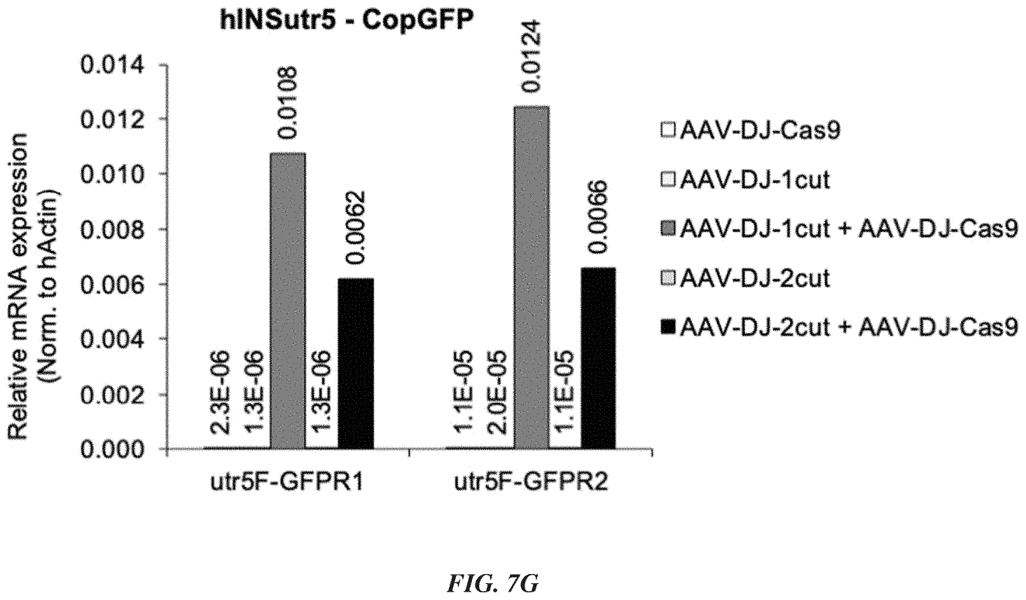

Previous reports of AAV transduction of human islets have shown limited success (Rehman et al., Gene Ther., 12:1313-1323 (2005) and Craig et al., Virol. J., 6:61 (2009), both of which are hereby incorporated by reference in their entirety). However, the success in using AAV to edit the insulin gene in the mouse pancreas (FIGS. 5B-5E) motivated the evaluation of various serotypes of AAV for their ability to deliver CRBR components into human islets and edit the human insulin gene. GFP overexpressing AAV serotypes 2, 5, 6, 8, 9, EB, and DJ were tested for their ability to transduce human islets, and found that AAV-DJ infection led to the most GFP-positive cells. To test the ability of CRBR-mediated gene editing in human islets ex vivo via AAV-DJ transduction, human islets were co-infected with AAV-DJ-sgRNA-CDS-1cut (or 2cut) (FIG. 7A) along with AAV-DJ-Cas9. CopGFP-positive cells were observed at 6-day post-infection (FIG. 7B). By 10- and 16-day post-infection, these cells dramatically increased in both number and fluorescence intensity (FIG. 7B). This indicates that living and functional human beta cells can at least maintain insulin expression for 16 days. When CRBR integration was analyzed at the genome level, the expected 5′ junction diagnostic PCR was observed with 3.21% (1-cut) or 0.75% (2-cut) of chromosome 11 edited, however, a few larger fragments were also amplified (FIG. 7C). DNA sequence analysis revealed that the larger fragments contained the left ITR and U6-driven hINS-in1 sgRNA cassette, which could still be spliced out, resulting in a wild-type 5′UTR for normal translation initiation. Single cell sorting of CRBR-treated human islets showed that 1.97% (1-cut strategy) or 0.96% (2-cut strategy) of the islet cells had undergone CopGFP integration and expression (FIG. 7D). By analyzing beta cell specific transcription factors (PDX1 and GLUT2), alpha cell specific (glucagon) and delta cell specific (somatostatin) markers, it was confirmed that the GFP positive cells were largely, if not exclusively, beta cells (FIGS. 7E-7F). The transcription of CopGFP from the human INS locus in an independent batch of human islets was measured 18-day post-infection (FIG. 7G), with that of the one-cut strategy slightly exceeding that of the two-cut strategy. The consistent better performance of one-cut strategy when looking at CRBR integration efficiency at the genome level, the fraction of GFP positive cell, and GFP mRNA expression suggests that using the 1-cut donor is more efficient than the 2-cut donor via AAV transduction. The second cut downstream of the cassette donor is not necessary because AAV vector does not have a large backbone as in plasmid vector. In conclusion, these results indicate CRBR-mediated gene editing via AAV transduction works effectively with human host DNA repair machinery and that AAV serotype DJ is a promising candidate vector for gene therapy in human pancreatic beta cells.

### Discussion of Examples 1-4

Delivering CRISPR-based therapeutics has been the favored approach for targeted gene correction in vivo in mitotically active tissues. Studies (Canny et al., Nat. Biotechnol., 36:95-102 (2018) and Nishiyama et al., Neuron, 96:755-768 (2017), both of which are hereby incorporated by reference in their entirety) aimed at improving efficiency of HDR in post-mitotic cells offer one solution, however the NHEJ-based repair pathway has provided an alternative strategy that is feasible in both mitotic and post-mitotic cells. Three groups independently (Long et al., Science (New York, N.Y.), 351:400-403 (2016); Nelson et al., Science (New York, N.Y.), 351:403-407 (2016); and Tabebordbar et al., Science (New York, N.Y.), 351:407-411 (2016), all of which are hereby incorporated by reference in their entirety) employed a NHEJ-based strategy to excise an exon of the Duchenne muscular dystrophy gene (Dmd) containing a deleterious mutation, which reversed muscular dystrophy in mice. However, the Dmd gene is atypical in its tolerance for exon loss, therefore, this strategy cannot be generalized to most other mutations. Suzuki and coworkers (Suzuki et al., Cell Research, 29:804-819 (2019), which is hereby incorporated by reference in its entirety) had recently developed a “intercellular linearized Single homology Arm donor mediated intron-Targeting Integration (SATI)”, which has great applications for targeting of a broad range of mutations and cell types by utilizing both NHEJ and HDR pathways. However, SATI strategy also requires a specific design for each mutation variant. Consequently, a gene editing strategy that can repair a spectrum of mutations without requiring the design and testing of a specific repair template for each mutation remains highly desirable.

The preceding examples demonstrates that the described CRBR strategy can be generalized to different kinds of monogenic diseases where traditional treatments or current gene therapy are not feasible or practical. The complete wild-type CDS used in CRBR strategy targets a non-coding region between the promoter and the downstream mutated region, thereby bypassing any mutation that may exist in the coding sequence. Once validated, the CRBR repair cassette should be able to rescue any deleterious or loss-of-function mutation that might exist in that gene. Currently, the efficiency of CRBR may be too low to directly repair genetic diseases systemically in humans where a large fraction of an organ or tissue may require repair to restore normal function. A more direct intra-organ injection route may improve the delivery to the pancreas or other tissues that are challenging to target by intravenous injection. Mutations in Perk, which result in severe and permanent neonatal diabetes in Wolcott-Rallison syndrome (WRS) patients, present a particularly difficult challenge because very few beta cells exist due to a severe postnatal cell proliferation defect (Zhang et al., Cell Metabolism, 4:491-497 (2006), which is hereby incorporated by reference in its entirety) and a block in proinsulin trafficking and processing (Sowers et al., The Journal of Biological Chemistry, 293:5134-5149 (2018), which is hereby incorporated by reference in its entirety). Consequently, there may not be enough beta cells present in a WRS patient's islets to repair. A more promising route would be to derive patient specific induced pluripotent stems cells (PS-iPSCs) from a WRS patient, perform CRBR gene repair, screen for CRBR corrected PS-iPSCs, and differentiate them into functional beta cells using the Maxwell protocol (Maxwell et al., Sci. Transl. Med., 12:540 (2020), which is hereby incorporated by reference in its entirety). These beta cells could then be transplanted back into the original patient. Repairing a defective gene in a patient's own cells would avoid transplantation rejection and the need for immunosuppressive drugs. Overall, CRBR gene repair combined with autologous cell replacement therapy (GR-ACR) should be generally applicable to a wide range of human genetic diseases.

While CRBR gene repair offers significant advantages, there are potential pitfalls that must be considered in the design and execution. Because CRBR relies upon the error-prone NHEJ repair pathway, small indels at the integration site of the CRBR cassette are common. It is therefore important to restrict the integration site to non-coding and non-regulatory sequences. Ideally, the integration site should be either in the 5′UTR or within an intron upstream of the coding sequence of the subject gene. The introduction of translational start codons or strong secondary mRNA structure in the 5′ UTR and alternative splice sites in an intron must also be avoided. However, because the nature of the indels at the integration site cannot be predetermined, mutations may be generated that result in alternative translational and splicing regulatory sequences that interfere with normal gene expression. It has been found that a small set of specific indels will be generated for any given CRISPR-Cas9 experiment. Therefore, testing the design in cell culture first can help identify the specific array and frequency of indels that are likely to occur. If necessary, the design may be modified to avoid mutations that interfere with gene expression and regulation. Alternatively, if a GR-ACR strategy is used, a specific cell line can be clonally isolated that is devoid of interfering mutations.

Although other delivery methods (Wilbie et al., Acc. Chem. Res., 52:1555-1564 (2019) and Yin et al., Nat. Biotechnol., 35:1179-1187 (2017), both of which are hereby incorporated by reference in their entirety) can be used, rAAV vectors are currently the safest delivery vectors for in vivo genome editing. However, AAV vectors have a limited packaging capacity of 4 kb. The CRBR strategy, which necessitates delivery of a large multi-element cassette (5′UTR/intronic sequences, CDS with stop codon, and heterologous polyA signal/transcriptional terminator), will be constrained by this size limitation for viral packaging as well as genomic integration efficiency. Fortunately, about 95% of human proteins are encoded by genes that are less than 4 kb. For genes that exceed 4 kb, a partial CRBR CDS can be designed for integration into introns upstream of the defective coding exons. Whether or not the integration of a partial CDS cassette will provide a general solution for repairing a spectrum of mutations that exist among patients with a genetic disease depends upon the distribution of the mutations across the coding sequence. An additional limitation of using rAAV vectors for CRISPR based gene editing is the persistent expression of Cas9 which may result in mutagenic and immunological complications (Ates et al., Genes (Basel), 11:(2020), which is hereby incorporated by reference in its entirety). To mitigate this problem, Cas9 mRNA or protein could be delivered by a non-viral vector along with the CRBR cassette and sgRNA delivered by an AAV vector. Alternatively, a self-deleting Cas9 could be employed to limit the expression of Cas9 (Li et al., Mol. Ther. Methods. Clin. Dev., 12:111-122 (2019), which is hereby incorporated by reference in its entirety).

To reduce the size of the CRBR repair cassette, the intronic sequences separating the CDS exons are excluded. However, this approach could be problematic for rare cases where alternative spliced transcripts are essential for normal gene function. In addition, important transcriptional regulatory elements such as enhancers may exist within intronic sequences and would be absent in the CRBR CDS-terminator cassette. In most cases, this should not pose a problem since these cis-acting regulatory elements would still exist downstream in the endogenous mutant gene and could still potentially serve to regulate gene transcription. As with all gene therapy strategies, thorough testing of repair efficacy in cell culture and/or model organisms is essential. A distinct advantage of CRBR gene correction strategy is that testing and validation need only be performed for a single design which can then be used to repair a spectrum of mutations among a population of human patients, thus substantially reducing the cost of treatment.

### SEQUENCE LISTING

Submitted with this application is a Sequence Listing in the form of an ASCII text (.txt) file, which is hereby incorporated by reference into the specification of the application. The ASCII text file (18 KB) was created on Jun. 17, 2022 and has the file name Sequence_Listing_148411_001701_ST25.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. All of the features described herein (including any accompanying claims, abstract and drawings), and/or all of the steps of any method or process so disclosed, may be combined with any of the above aspects in any combination, except combinations where at least some of such features and/or steps are mutually exclusive. Additionally, the recited order of processing elements or sequences, or the use of numbers, letters, or other designations therefore, is not intended to limit the claimed processes to any order except as may be specified in the claims. These alterations, improvements, and modifications are intended to be suggested hereby, and are within the spirit and scope of the invention. Accordingly, the invention is limited only by the following claims and equivalents thereto.

1. A method of correcting a gene defect in a cell comprising:
providing in a cell having a gene defect (i) a chimeric Clustered Regularly Interspaced Short Palindromic Repeats (CRISPR) associated (Cas) protein or a first nucleic acid molecule encoding the Cas protein, (ii) a guide RNA that is capable of base-pairing with a region of the defective gene between a promoter and a coding sequence thereof, or a second nucleic acid encoding the guide RNA, and (iii) a DNA template comprising a replacement coding sequence, which encodes a non-defective protein, and a transcription terminator sequence,
wherein upon binding of the guide RNA to the 5′ untranslated region of the defective gene and cleavage of the 5′ untranslated region by the Cas protein, the DNA template is inserted into the genome of the cell via non-homologous end-joining (NHEJ) repair pathway to allow for expression of the non-defective protein under control of the promoter while simultaneously blocking the expression of the defective gene, thereby correcting the gene defect.

2. (canceled)

3. The method according to claim 1, wherein said providing or said repairing is carried out by introducing into the cell one or more vectors comprising the first nucleic acid molecule, the second nucleic acid molecule, and the DNA template.

4. The method according to claim 3, wherein the one or more vectors comprise one or more viral vectors selected from the group consisting of adeno-associated virus, adenovirus, and lentivirus vectors.

5. (canceled)

6. The method according to claim 1, wherein said providing or said repairing is carried out by introducing into the cell one or more non-viral delivery vehicles comprising the Cas protein or mRNA encoding the Cas protein, the guide RNA, and the DNA template.

7. The method according to claim 6, wherein the non-viral delivery vehicle comprises a lipid-like nanoparticle, inorganic nanoparticle, cell-penetrating peptide, DNA nanoclew, cationic nanocarrier, zeolitic imidazole framework, zwitterionic amino-lipid nanoparticles, or antibody tissue-targeting.

8. The method according to claim 1, wherein said introducing is carried out by microinjection, electroporation, or hydrodynamic injection.

9. (canceled)

10. The method according to claim 1, wherein the cell is ex vivo.

11. The method according to claim 1, wherein the cell is a mitotic or post-mitotic cell.

12. The method according to claim 10 wherein the cell is a pluripotent stem cell, a somatic stem cell, a de-differentiated cell, or a zygote.

13. The method according to claim 10, further comprising obtaining the cell from an individual prior to said providing or from the patient prior to said repairing.

14. (canceled)

15. The method according to claim 1, further comprising:
selecting cells having corrected the gene defect; and
introducing selected cells into the individual.

16. The method according to claim 15, wherein said selecting further comprises selecting cells that also lack insertions or deletions at the replacement coding sequence integration site.

17. The method according to claim 15 further comprising isolating the selected cells and culturing the isolated cells to prior to introducing.

18. The method according to claim 1, wherein the coding sequence of the DNA template is intronless.

19. The method according to claim 1, wherein the coding sequence of the DNA template comprises one or more introns.

20. (canceled)

21. The method according to claim 1, wherein the target region where the guide RNA binds is a 5′ untranslated region of the defective gene or within an intron located 5′ of the defective gene coding sequence.

22. The method according to claim 1, wherein the Cas protein is a Cas9 protein selected from Streptococcus pyogenes Cas9 and Streptococcus aureus Cas9.

23. (canceled)

24. The method according to claim 1, wherein the guide RNA comprises one or more modified bases or a modified backbone.

25. The method according to claim 1, wherein the non-defective protein is a wild-type variant or a modified variant having improved activity relative to wild-type.

26.-30. (canceled)

31. The method according to claim 1, wherein the DNA template further comprises an identical or nearly identical nucleotide sequence as the target binding site.

32.-86. (canceled)
